(19) United States
(12) Patent Application Publication
(10) Pub. No.: US 2011/0195854 A1
(43) Pub. Date: Aug. 11, 2011

(54) DETECTING AND MONITORING INFLAMMATORY NEUROPATHY

(75) Inventors: Norman Latov, Irvington, NY (US); Grace Lee, Riverdale, NY (US)

(73) Assignee: Cornell Research Foundation, Inc., Ithaca, NY (US)

(21) Appl. No.: 12/702,693

(22) Filed: Feb. 9, 2010

(51) Int. Cl. C40B 30/04; C12Q 1/68; G01N 33/53; C12Q 1/02

(52) U.S. Cl. 506/9; 435/7.1; 435/29; 435/6.11; 435/6.12; 435/5

(57) ABSTRACT

The invention relates to detection and/or monitoring of inflammatory neuropathy using markers that specifically indicate the presence of inflammatory neuropathy, for example, allograft inflammatory factor 1 (AIF1), lymphatic hyaluronan receptor (LYVE-1), FYN binding protein (FYB), myeloid/lymphoid or mixed-lineage leukemia, translocated to, 3 (MLLT3), purinergic receptor P2Y, G-protein coupled, 1 (P2RY1) or a combination thereof. According to the invention, skin biopsies can be used for assessing the expression of these markers.

### RELATED APPLICATIONS

This application is a national stage application under 35 U.S.C. §111(a) of PCT/US2008/009544, filed Aug. 8, 2008, and published as WO 2009/023140 on Feb. 19, 2009, which claims priority to the filing date of U.S. Provisional Application Ser. No. 60/955,140, filed Aug. 10, 2008, the contents of which are specifically incorporated by reference herein in their entirety.

This application also related to U.S. application Ser. No. 11/363,151, filed Feb. 28, 2006, and claiming benefit of the filing date of U.S. Provisional Application Ser. No. 60/657,122, filed Feb. 28, 2005. In addition, this application is related to U.S. application Ser. No. 11/363,149, filed Feb. 28, 2006, and also claiming benefit of the filing date of U.S. Provisional Application Ser. No. 60/657,122, filed Feb. 28, 2005. The disclosures of U.S. application Ser. Nos. 11/363,151, 11/363,149 and 60/657,122 are specifically incorporated herein by reference in their entireties.

### SEQUENCE LISTING

This application includes a nucleotide and/or amino acid sequence listing that is being electronically filed with the application as an ASCII-compliant text file, which is named SequenceListing.txt, created on Feb. 9, 2010, and which is 52 KB in size.

### FIELD OF THE INVENTION

The invention relates to detecting and monitoring inflammatory neuropathies (e.g., chronic inflammatory demyelinating polyneuropathy (CIDP)) by observing altered expression of allograft inflammatory factor (AIF1), lymphatic hyaluronan receptor (LYVE-1) and FYN binding protein (FYB), myeloid/lymphoid or mixed-lineage leukemia, translocated to, 3 (MLLT3) and/or purinergic receptor P2Y, G-protein coupled, 1 (P2RY1). In some embodiments, the biological sample used for detecting the inflammatory neuropathy is a skin biopsy.

### BACKGROUND

Chronic inflammatory demyelinating polyradiculoneuropathy (CIDP) is an autoimmune disease that targets myelin sheaths, specifically in the peripheral nerves, and causes progressive weakness and sensory loss. Vasculitis is caused by inflammation of the blood vessel walls. When the blood vessels in the nerves are affected, it is referred to as vasculitic neuropathy.

Both CIDP and vasculitic neuropathy cause peripheral neuropathy which is manifest by sensory loss, weakness, or pain, alone or in combination, in the arms, legs, or other parts of the body. Both can cause a symmetric or multifocal neuropathy and affect the proximal or distal muscles. There are many other causes of neuropathy besides CIDP and vasculitis, but in one quarter to one third of neuropathies, no cause can be found, and the neuropathy is called idiopathic. This is due, in part, to the lack of reliable tests for many causes of neuropathy.

CIDP is currently diagnosed based on the clinical presentation, evidence for demyelination on electrodiagnostic studies or pathological studies of biopsied nerves, and elimination of other known causes of neuropathy such as genetic defects, osteosclerotic myeloma, or IgM monoclonal gammopathy. There is currently no definitive test, and the diagnosis can be missed, especially in atypical cases or in sensory CIDP where the electrodiagnostic tests are less reliable. Such cases may be difficult to distinguish from vasculitic neuropathy. Nerve biopsy is done in cases where the diagnosis is uncertain, but its usefulness is limited by its relative insensitivity and the need for quantitative morphological analysis which is only available in a small number of academic centers. For further discussions about properties of, or current diagnostic methods for, CIDP, see, e.g., Dyck et al. (1975) Mayo Clin. Proc. 50, 621-637; Latov (2002) Neurology 59, S2-S6; Berger et al. (2003) J. Peripher. Nerv. Sys. 8, 282-284; Ad Hoc Subcommittee of the AAN (1991); Barohn et al. (1989) Arch. Neurol. 46, 878-884; Bouchard et al. (1999) Neurology 52, 498-503).

Thus, improved methods for detecting and diagnosing inflammatory neuropathies such as CIDP are needed.

### SUMMARY OF THE INVENTION

This disclosure demonstrates by quantitative real-time PCR (RT-PCR) and gene microarray profiling analyses that several markers in skin biopsies can be used to aid in the diagnosis and/or screening of patients with neuropathies, including the markers allograft inflammatory factor (AIF1), lymphatic hyaluronan receptor (LYVE-1), FYN binding protein (FYB), myeloid/lymphoid or mixed-lineage leukemia, translocated to, 3 (MLLT3) and/or purinergic receptor P2Y, G-protein coupled, 1 (P2RY1). In some embodiments, neuropathies can be detected and/or diagnosed by detecting one of these markers. In other embodiments, neuropathies are detected and/or diagnosed using a combination of any of these markers.

Thus, one aspect of the invention is a method of detecting or monitoring inflammatory neuropathy in a patient comprising: (a) obtaining a biological sample from the patient; (b) comparing a test expression level of allograft inflammatory factor 1 (AIF1), lymphatic hyaluronan receptor (LYVE-1), FYN binding protein (FYB), myeloid/lymphoid or mixed-lineage leukemia, translocated to, 3 (MLLT3), purinergic receptor P2Y, G-protein coupled, 1 (P2RY1) or a combination thereof, in the biological sample, with a control expression level of allograft inflammatory factor 1 (AIF1), lymphatic hyaluronan receptor (LYVE-1), FYN binding protein (FYB), myeloid/lymphoid or mixed-lineage leukemia, translocated to, 3 (MLLT3), purinergic receptor P2Y, G-protein coupled, 1 (P2RY1), or a combination thereof; and (c) detecting inflammatory neuropathy when the test expression level is at least 1.5-fold greater than the control expression level.

The term “detecting” inflammatory neuropathy means that elevated levels of markers (e.g., AIF1, FYB, LYVE-1, MLLT3, P2RY1 or combinations thereof) have been observed. As demonstrated herein, AIF1, FYB, LYVE-1, MLLT3 and P2RY1 are markers for inflammatory neuropathy because elevation in their expression is tightly correlated with the presence of an inflammatory neuropathy condition. Moreover, in some instances, “detecting” can also mean that an inflammatory neuropathy has not yet been detected in a specific patient. In other instances, “detecting” means that while inflammatory neuropathy has previously been detected in a specific patient, the patient is being re-evaluated to ascertain whether relapse, progression or regression of the inflammatory neuropathy is occurring. Thus, “detecting” inflammatory neuropathy also includes “monitoring” inflammatory neuropathy. “Monitoring” means that the degree to which a patient still suffers from inflammatory neuropathy is being evaluated. When monitoring inflammatory neuropathy the patient is being re-evaluated to ascertain whether relapse, progression or regression of the inflammatory neuropathy is occurring.

While a variety of biological samples can be employed, in some embodiments, the biological sample is a skin biopsy. In other embodiments, the biological sample is a nerve biopsy.

The methods of the invention can be used to detect or monitor a variety of inflammatory neuropathies. For example, the inflammatory neuropathy can be an infectious neuropathy or an autoimmune neuropathy. In other embodiments, the inflammatory neuropathy can be Lyme disease, HIV infection, AIDS, Leprosy, Herpes Zoster (Shingles), Hepatitis B infection, Hepatitis C infection, an autoimmune disease, Sarcoidosis, Guillain-Barré Syndrome, Acute Inflammatory Demyelinating Polyneuropathy (AIDP), Chronic Inflammatory Demyelinating Polyneuropathy (CIDP), Vasculitis, Polyarteritis Nodosa (PAN), Rheumatoid Arthritis, Systemic Lupus Erythematosus, Sjögren's Syndrome, Celiac Disease, Multifocal Motor Neuropathy (MNN), Peripheral Neuropathy Associated with Protein Abnormalities, Monoclonal Gammopathy, Amyloidosis, Cryoglobulinemia and/or POEMS, or a combination thereof. In further embodiments, the inflammatory neuropathy is chronic inflammatory demyelinating polyneuropathy (CIDP).

The control expression levels can be expression levels of allograft inflammatory factor 1 (AIF1), lymphatic hyaluronan receptor (LYVE-1), FYN binding protein (FYB), myeloid/lymphoid or mixed-lineage leukemia, translocated to, 3 (MLLT3), purinergic receptor P2Y, G-protein coupled, 1 (P2RY1) or a combination thereof, in a biological sample from a healthy patient who does not have inflammatory neuropathy. In some embodiments, the control expression levels are expression levels of allograft inflammatory factor 1 (AIF1), lymphatic hyaluronan receptor (LYVE-1), FYN binding protein (FYB), myeloid/lymphoid or mixed-lineage leukemia, translocated to, 3 (MLLT3), purinergic receptor P2Y, G-protein coupled, 1 (P2RY1), or a combination thereof, in a biological sample from a patient with non-inflammatory neuropathy. In other embodiments, the control expression levels are expression levels of allograft inflammatory factor 1 (AIF1), lymphatic hyaluronan receptor (LYVE-1), FYN binding protein (FYB), myeloid/lymphoid or mixed-lineage leukemia, translocated to, 3 (MLLT3), purinergic receptor P2Y, G-protein coupled, 1 (P2RY1), or a combination thereof, in a biological sample from a patient with hereditary demyelinating neuropathy, Charcot-Marie-Tooth disease type I (CMT1), or diabetic neuropathy (DN).

Inflammatory neuropathy can be detected or diagnosed, for example, when the lymphatic hyaluronan receptor (LYVE-1) expression level in the biological sample is about 2 to about 3 fold greater than the control lymphatic hyaluronan receptor (LYVE-1) expression levels.

Inflammatory neuropathy can also be detected or diagnosed when the allograft inflammatory factor 1 (AIF1) expression level in the biological sample is, for example, about 2 to about 8 fold greater than the control allograft inflammatory factor 1 (AIF1) expression levels.

Moreover, inflammatory neuropathy can also be detected or diagnosed when the FYN binding protein (FYB) expression level in the biological sample is about 1.5 to about 3 fold greater than the control FYN binding protein (FYB) expression level.

In addition, inflammatory neuropathy is detected or diagnosed when the purinergic receptor P2Y, G-protein coupled, 1 (P2RY1) expression level in the biological sample is about 1.5 to about 3 fold greater than the control purinergic receptor P2Y, G-protein coupled, 1 (P2RY1) expression level.

Moreover, inflammatory neuropathy is also detected or diagnosed when the myeloid/lymphoid or mixed-lineage leukemia, translocated to, 3 (MLLT3) expression level in the biological sample is about 1.5 to about 3 fold greater than the control myeloid/lymphoid or mixed-lineage leukemia, translocated to, 3 (MLLT3) expression level.

Test expression levels and control expression levels can, for example, be determined by a quantitative real time polymerase chain reaction (qPCR) assay. Primers for the quantitative real time polymerase chain reaction assay are available that selectively hybridize to any of SEQ ID NO:1, 3, 5, 7, 9, 11, 13, 14, or a combination thereof, for example, under stringent hybridization conditions.

In other embodiments, the test expression levels and control expression levels are determined by quantitative microarray analysis. Probes used for the quantitative microarray analysis can, for example, selectively hybridize to any of SEQ ID NO:1, 3, 5, 7, 9, 11, 13, 14, or a combination thereof, under stringent hybridization conditions.

In further embodiments, the test expression levels and control expression levels are determined by quantitative RNA hybridization assay. For example, probes for the quantitative RNA hybridization assay can be identified as having any of SEQ ID NO:1, 3, 5, 7, 9, 11, 13, 14, or a combination thereof, or being complementary thereto. In some instances, such probes can selectively hybridize to any of SEQ ID NO:1, 3, 5, 7, 9, 11, 13, 14, or a combination thereof, under stringent hybridization conditions.

In still further embodiments, the test expression levels and control expression levels are determined by quantitative northern hybridization assay, for example, using probes or primers that are derived from or are complementary to any of SEQ ID NO:1, 3, 5, 7, 9, 11, 13, 14, or a combination thereof, where the northern hybridization assay is performed using stringent hybridization conditions.

Another aspect of the invention is a method of detecting or monitoring inflammatory neuropathy in a patient comprising: (a) obtaining a test skin biopsy from a patient; (b) quantifying expression of lymphatic hyaluronan receptor (LYVE-1) in the test skin biopsy to obtain quantitative test expression levels of lymphatic hyaluronan receptor (LYVE-1); (c) determining whether the quantitative test expression levels are greater than quantitative control expression levels of lymphatic hyaluronan receptor (LYVE-1) in a control skin biopsy; and (d) detecting inflammatory neuropathy when the quantitative test expression levels are at least 2-fold greater than the quantitative control expression levels.

Control biological samples can be samples (e.g., skin biopsy samples or nerve biopsy samples) from persons that do not suffer from inflammatory neuropathies, especially chronic inflammatory demyelinating polyradiculoneuropathy (CIDP). In some embodiments, the control skin biopsy is a skin biopsy of a normal patient who does not have inflammatory neuropathy. In other embodiments, the control skin biopsy is a skin biopsy of a patient with non-inflammatory neuropathy. In further embodiments, the control skin biopsy is a skin biopsy of a patient with hereditary demyelinating neuropathy, Charcot-Marie-Tooth disease type I (CMT1), or diabetic neuropathy (DN).

Quantifying expression of lymphatic hyaluronan receptor (LYVE-1) can be done by any available method. In some embodiments, LYVE-1 probes or primers are selected from, or complementary to, a region of SEQ ID NO:1 or 3. Such probes can selectively hybridize to a region of SEQ ID NO:1 or 3, for example, under stringent hybridization conditions. The same LYVE-1 probes or primers can be used for quantifying control expression levels of lymphatic hyaluronan receptor (LYVE-1) in a control skin biopsy, for example, under stringent hybridization conditions.

Another aspect of the invention is a method of detecting or monitoring chronic inflammatory demyelinating polyradiculoneuropathy (CIDP) in a patient comprising: (a) obtaining a skin biopsy from the patient; (b) comparing a test expression level of allograft inflammatory factor 1 (AIF1), lymphatic hyaluronan receptor (LYVE-1), FYN binding protein (FYB), myeloid/lymphoid or mixed-lineage leukemia, translocated to, 3 (MLLT3), purinergic receptor P2Y, G-protein coupled, 1 (P2RY1) or a combination thereof, in the skin biopsy, with a control expression level of allograft inflammatory factor 1 (AIF1), lymphatic hyaluronan receptor (LYVE-1), FYN binding protein (FYB), myeloid/lymphoid or mixed-lineage leukemia, translocated to, 3 (MLLT3), purinergic receptor P2Y, G-protein coupled, 1 (P2RY1), or a combination thereof, in a control sample from a patient with hereditary demyelinating neuropathy; and (c) detecting chronic inflammatory demyelinating polyradiculoneuropathy (CIDP) when the test expression level is at least 1.5-fold greater than the control expression level.

### DESCRIPTION OF THE FIGURES

FIG. 1A-B shows data confirming by qPCR (black bars) the gene expression levels detected by microarray (gray bars) for AIF1, FYB, LYVE-1, MLLT3 and P2RY1. qPCR results for CIDP (FIG. 1A) are significantly different from CMT1 (FIG. 1B) at the p values given under the gene name. Each gene in the patient groups is normalized to the Normal group value (normal is FC=1). * The Aver. Index is the group average of the sum for all 5 genes for CIDP or CMT1. The difference between the indexes is significant at p=0.0018.

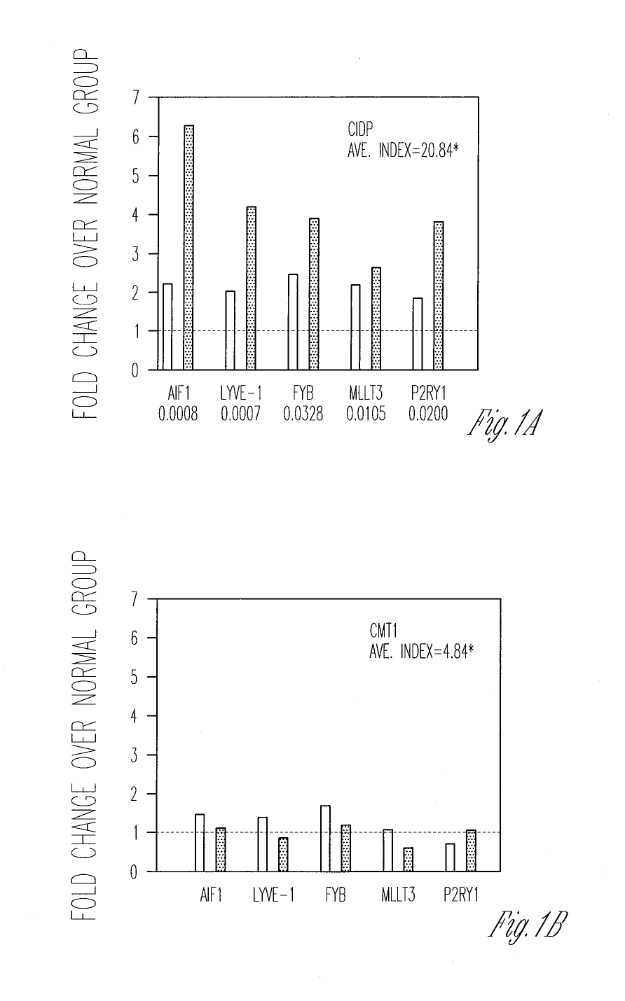

### DETAILED DESCRIPTION OF THE INVENTION

The invention relates to detection and monitoring of inflammatory neuropathies in patients by observing whether increased expression levels of allograft inflammatory factor (AIF1), lymphatic hyaluronan receptor (LYVE-1), FYN binding protein (FYB), myeloid/lymphoid or mixed-lineage leukemia, translocated to, 3 (MLLT3) and/or purinergic receptor P2Y, G-protein coupled, 1 (P2RY1) are present in skin biopsies obtained from the patients. Surprisingly, skin biopsies can be used in the inventive procedures rather than nerve biopsies. Skin biopsies are not only easier and less invasive to obtain, but they also avoid the dangers associated with obtaining nerve biopsies.

Gene microarray analysis of sural nerve biopsies have previously shown that a set of molecular markers, including AIF1 (allograft inflammatory factor) and CLCA2 (chloride channel, calcium activated, family member 2), are elevated in patients with vasculitic neuropathy or chronic demyelinating polyneuropathy (CIDP) (Renaud et al, 2005). However, obtaining nerve biopsies requires invasive procedures that are not only unpleasant but can also be dangerous.

Expression of AIF1, LYVE-1, FYB, MLLT3 and/or P2RY1 can be detected by any procedure available in the art.

In some embodiments, expression of AIF1, LYVE-1, FYB, MLLT3 and/or P2RY1, and combinations thereof, are detected by measuring RNA expression levels quantitatively. For example, RNA levels can be detected by quantitative microarray expression analysis, Northern blot analysis or real time-polymerase chain reaction (RT-PCR). Procedures for performing quantitative microarray expression analysis, Northern blot and RT-PCR analyses are available in the art, and are described in more detail below.

Any selective probe for performing Northern blot analysis or microarray analysis, or set of selective primers for performing quantitative RT-PCR, on RNA samples from skin biopsies for detecting AIF1, LYVE-1, FYB, MLLT3 and/or P2RY1 can be employed. Such primers can be selected by examination of nucleic acid sequences for AIF1, LYVE-1, FYB, MLLT3 and/or P2RY1. Nucleic acid sequences for AIF1, LYVE-1, FYB, MLLT3 and/or P2RY1 can be found in the art, for example, in the National Center for Biotechnology Information (NCBI) database. See website at ncbi.nlm.nih.gov.

In general, as used herein, the terms “nucleic acid” and “polynucleotide” are used interchangeably to refer to a DNA or RNA molecule, including a genomic DNA, cDNA, mRNA, probe, primer, DNA fragment, RNA fragment, or the like.

### Lymphatic Vessel Endothelial Hyaluronan Receptor 1 (LYVE1)

For example, one nucleotide sequence for Homo sapiens lymphatic vessel endothelial hyaluronan receptor 1 (LYVE1), mRNA is available in the NCBI database as accession number NM 006691 (gi: 151301201). See website at ncbi.nlm.nih.gov. This sequence is provided below as follows (SEQ ID NO:1).

| | |
|---|---|
| 1 | CTCATTTGTG TGTTTTCTGA GTCAGCATTA GCTAAATTTT |
| 41 | CCAGAAGGCC ATCCACAAAG TACAGCCTGG GCGTTCAAGG |
| 81 | GACGTCATTC ATTTCCCCCA GTGACCTTGA CAAGTCAGAA |
| 121 | GCTTGAAAGC AGGGAAATCC GGATGTCTCG GTTATGAAGT |
| 161 | GGAGCAGTGA GTGTGAGCCT CAACATAGTT CCAGAACTCT |
| 201 | CCATCCGGAC TAGTTATTGA GCATCTGCCT CTCATATCAC |
| 241 | CAGTGGCCAT CTGAGGTGTT TCCCTGGCTC TGAAGGGGTA |
| 281 | GGCACGATGG CCAGGTGCTT CAGCCTGGTG TTGCTTCTCA |
| 321 | CTTCCATCTG GACCACGAGG CTCCTGGTCC AAGGCTCTTT |
| 361 | GCGTGCAGAA GAGCTTTCCA TCCAGGTGTC ATGCAGAATT |
| 401 | ATGGGGATCA CCCTTGTGAG CAAAAAGGCG AACCAGCAGC |
| 441 | TGAATTTCAC AGAAGCTAAG GAGGCCTGTA GGCTGCTGGG |
| 481 | ACTAAGTTTG GCCGGCAAGG ACCAAGTTGA AACAGCCTTG |
| 521 | AAAGCTAGCT TTGAAACTTG CAGCTATGGC TGGGTTGGAG |
| 561 | ATGGATTCGT GGTCATCTCT AGGATTAGCC CAAACCCCAA |
| 601 | GTGTGGGAAA AATGGGGTGG GTGTCCTGAT TTGGAAGGTT |
| 641 | CCAGTGAGCC GACAGTTTGC AGCCTATTGT TACAACTCAT |
| 681 | CTGATACTTG GACTAACTCG TGCATTCCAG AAATTATCAC |
| 721 | CACCAAAGAT CCCATATTCA ACACTCAAAC TGCAACACAA |
| 761 | ACAACAGAAT TTATTGTCAG TGACAGTACC TACTCGGTGG |
| 801 | CATCCCCTTA CTCTACAATA CCTGCCCCTA CTACTACTCC |
| 841 | TCCTGCTCCA GCTTCCACTT CTATTCCACG GAGAAAAAAA |
| 881 | TTGATTTGTG TCACAGAAGT TTTTATGGAA ACTAGCACCA |
| 921 | TGTCTACAGA AACTGAACCA TTTGTTGAAA ATAAAGCAGC |
| 961 | ATTCAAGAAT GAAGCTGCTG GGTTTGGAGG TGTCCCCACG |
| 1001 | GCTCTGCTAG TGCTTGCTCT CCTCTTCTTT GGTGCTGCAG |
| 1041 | CTGGTCTTGG ATTTTGCTAT GTCAAAAGGT ATGTGAAGGC |
| 1081 | CTTCCCTTTT ACAAACAAGA ATCAGCAGAA GGAAATGATC |
| 1121 | GAAACCAAAG TAGTAAAGGA GGAGAAGGCC AATGATAGCA |
| 1161 | ACCCTAATGA GGAATCAAAG AAAACTGATA AAAACCCAGA |
| 1201 | AGAGTCCAAG AGTCCAAGCA AAACTACCGT GCGATGCCTG |
| 1241 | GAAGCTGAAG TTTAGATGAG ACAGAAATGA GGAGACACAC |
| 1281 | CTGAGGCTGG TTTCTTTCAT GCTCCTTACC CTGCCCCAGC |
| 1321 | TGGGGAAATC AAAAGGGCCA AAGAACCAAA GAAGAAAGTC |
| 1361 | CACCCTTGGT TCCTAACTGG AATCAGCTCA GGACTGCCAT |
| 1401 | TGGACTATGG AGTGCACCAA AGAGAATGCC CTTCTCCTTA |
| 1441 | TTGTAACCCT GTCTGGATCC TATCCTCCTA CCTCCAAAGC |
| 1481 | TTCCCACGGC CTTTCTAGCC TGGCTATGTC CTAATAATAT |
| 1521 | CCCACTGGGA GAAAGGAGTT TTGCAAAGTG CAAGGACCTA |
| 1561 | AAACATCTCA TCAGTATCCA GTGGTAAAAA GGCCTCCTGG |
| 1601 | CTGTCTGAGG CTAGGTGGGT TGAAAGCCAA GGAGTCACTG |
| 1641 | AGACCAAGGC TTTCTCTACT GATTCCGCAG CTCAGACCCT |
| 1681 | TTCTTCAGCT CTGAAAGAGA AACACGTATC CCACCTGACA |
| 1721 | TGTCCTTCTG AGCCCGGTAA GAGCAAAAGA ATGGCAGAAA |
| 1761 | AGTTTAGCCC CTGAAAGCCA TGGAGATTCT CATAACTTGA |
| 1801 | GACCTAATCT CTGTAAAGCT AAAATAAAGA AATAGAACAA |
| 1841 | GGCTGAGGAT ACGACAGTAC ACTGTCAGCA GGGACTGTAA |
| 1881 | ACACAGACAG GGTCAAAGTG TTTTCTCTGA ACACATTGAG |
| 1921 | TTGGAATCAC TGTTTAGAAC ACACACACTT ACTTTTTCTG |
| 1961 | GTCTCTACCA CTGCTGATAT TTTCTCTAGG AAATATACTT |
| 2001 | TTACAAGTAA CAAAAATAAA AACTCTTATA AATTTCTATT |
| 2041 | TTTATCTGAG TTACAGAAAT GATTACTAAG GAAGATTACT |
| 2081 | CAGTAATTTG TTTAAAAAGT AATAAAATTC AACAAACATT |
| 2121 | TGCTGAATAG CTACTATATG TCAAGTGCTG TGCAAGGTAT |
| 2161 | TACACTCTGT AATTGAATAT TATTCCTCAA AAAATTGCAC |
| 2201 | ATAGTAGAAC GCTATCTGGG AAGCTATTTT TTTCAGTTTT |
| 2241 | GATATTTCTA GCTTATCTAC TTCCAAACTA ATTTTTATTT |
| 2281 | TTGCTGAGAC TAATCTTATT CATTTTCTCT AATATGGCAA |
| 2321 | CCATTATAAC CTTAATTTAT TATTAACATA CCTAAGAAGT |
| 2361 | ACATTGTTAC CTCTATATAC CAAAGCACAT TTTAAAAGTG |
| 2401 | CCATTAACAA ATGTATCACT AGCCCTCCTT TTTCCAACAA |
| 2441 | GAAGGGACTG AGAGATGCAG AAATATTTGT GACAAAAAAT |
| 2481 | TAAAGCATTT AGAAAACTTC AAAAAAAAAA AAAAAAAA |

The protein sequence for the mRNA is available in the NCBI database as accession number NP 006682 (gi: 40549451). See website at ncbi.nlm.nih.gov. This sequence is provided below as follows (SEQ ID NO:2).

| | |
|---|---|
| 1 | MARCFSLVLL LTSIWTTRLL VQGSLRAEEL SIQVSCRIMG |
| 41 | ITLVSKKANQ QLNFTEAKEA CRLLGLSLAG KDQVETALKA |
| 81 | SFETCSYGWV GDGFVVISRI SPNPKCGKNG VGVLIWKVPV |
| 121 | SRQFAAYCYN SSDTWTNSCI PEIITTKDPI FNTQTATQTT |
| 161 | EFIVSDSTYS VASPYSTIPA PTTTPPAPAS TSIPRRKKLI |
| 201 | CVTEVFMETS TMSTETEPFV ENKAAFKNEA AGFGGVPTAL |
| 241 | LVLALLFFGA AAGLGFCYVK RYVKAFPFTN KNQQKEMIET |
| 281 | KVVKEEKAND SNPNEESKKT DKNPEESKSP SKTTVRCLEA |
| 321 | EV |

Another sequence for Homo sapiens lymphatic vessel endothelial hyaluronan receptor 1 (LYVE1), mRNA is available in the NCBI database as accession number BC026231 (gi: 20070754). See website at ncbi.nlm.nih.gov. This sequence is provided below as follows (SEQ ID NO:3).

| | |
|---|---|
| 1 | AGTGGCCATC TGAGGTGTTT CCCTGGCTCT GAAGGGGTAG |
| 41 | GCACGATGGC CAGGTGCTTC AGCCTGGTGT TGCTTCTCAC |
| 81 | TTCCATCTGG ACCACGAGGC TCCTGGTCCA AGGCTCTTTG |
| 121 | CGTGCAGAAG AGCTTTCCAT CCAGGTGTCA TGCAGAATTA |
| 161 | TGGGGATCAC CCTTGTGAGC AAAAAGGCGA ACCAGCAGCT |
| 201 | GAATTTCACA GAAGCTAAGG AGGCCTGTAG GCTGCTGGGA |
| 241 | CTAAGTTTGG CCGGCAAGGA CCAAGTTGAA ACAGCCTTGA |
| 281 | AAGCTAGCTT TGAAACTTGC AGCTATGGCT GGGTTGGAGA |
| 321 | TGGATTCGTG GTCATCTCTA GGATTAGCCC AAACCCCAAG |
| 361 | TGTGGGAAAA ATGGGGTGGG TGTCCTGATT AGGAAGGTTC |
| 401 | CAGTGAGCCG ACAGTTTGCA GCCTATTGTT ACAACTCATC |
| 441 | TGATACTTGG ACTAACTCGT GCATTCCAGA AATTATCACC |
| 481 | ACCAAAGATC CCATATTCAA CACTCAAACT GCAACACAAA |
| 521 | CAACAGAATT TATTGTCAGT GACAGTACCT ACTCGGTGGC |
| 561 | ATCCCCTTAC TCTACAATAC CTGCCCCTAC TACTACTCCT |
| 601 | CCTGCTCCAG CTTCCACTTC TATTCCACGG AGAAAAAAAT |
| 641 | TGATTTGTGT CACAGAAGTT TTTATGGAAA CTAGCACCAT |
| 681 | GTCTACAGAA ACTGAACCAT TTGTTGAAAA TAAAGCAGCA |
| 721 | TTCAAGAATG AAGCTGCTGG GTTTGGAGGT GTCCCCACGG |
| 761 | CTCTGCTAGT GCTTGCTCTC CTCTTCTTTG GTGCTGCAGC |
| 801 | TGGTCTTGGA TTTTGCTATG TCAAAAGGTA TGTGAAGGCC |
| 841 | TTCCCTTTTA CAAACAAGAA TCAGCAGAAG GAAATGATCG |
| 881 | AAACCAAAGT AGTAAAGGAG GAGAAGGCCA ATGATAGCAA |
| 921 | CCCTAATGAG GAATCAAAGA AAACTGATAA AAACCCAGAA |
| 961 | GAGTCCAAGA GTCCAAGCAA AACTACCGTG CGATGCCTGG |
| 1001 | AAGCTGAAGT TTAGATGAGA CAGAAATGAG GAGACACACC |
| 1041 | TGAGGCTGGT TTCTTTCATG CTCCTTACCC TGCCCCAGCT |
| 1081 | GGGGAAATCA AAAGGGCCAA AGAACCAAAG AAGAAAGTCC |
| 1121 | ACCCTTGGTT CCTAACTGGA ATCAGCTCAG GACTGCCATT |
| 1161 | GGACTATGGA GTGCACCAAA GAGAATGCCC TTCTCCTTAT |
| 1201 | TGTAACCCTG TCTGGATCCT ATCCTCCTAC CTCCAAAGCT |
| 1241 | TCCCACGGCC TTTCTAGCCT GGCTATGTCC TAATAATATC |
| 1281 | CCACTGGGAG AAAGGAGTTT TGCAAAGTGC AAGGACCTAA |
| 1321 | AACATCTCAT CAGTATCCAG TGGTAAAAAG GCCTCCTGGC |
| 1361 | TGTCTGAGGC TAGGTGGGTT GAAAGCCAAG GAGTCACTGA |
| 1401 | GACCAAGGCT TTCTCTACTG ATTCCGCAGC TCAGACCCTT |
| 1441 | TCTTCAGCTC TGAAAGAGAA ACACGTATCC CACCTGACAT |
| 1481 | GTCCTTCTGA GCCCGGTAAG AGCAAAAGAA TGGCAGAAAA |
| 1521 | GTTTAGCCCC TGAAAGCCAT GGAGATTCTC ATAACTTGAG |
| 1561 | ACCTAATCTC TGTAAAGCTA AAATAAAGAA ATAGAACAAG |
| 1601 | GCTGAGGATA CGACAGTACA CTGTCAGCAG GGACTGTAAA |
| 1641 | CACAGACAGG GTCAAAGTGT TTTCTCTGAA CACATTGAGT |
| 1681 | TGGAATCACT GTTTAGAACA CACACACTTA CTTTTTCTGG |
| 1721 | TCTCTACCAC TGCTGATATT TTCTCTAGGA AATATACTTT |
| 1761 | TACAAGTAAC AAAAATAAAA ACTCTTATAA ATTTCTATTT |
| 1801 | TTATCTGAGT TACAGAAGTG ATTACTAAGG AAGATTACTC |
| 1841 | AGTAATTTGT TTAAAAAGTA ATAAAATTCA ACAAACATTT |
| 1881 | GCTGAATAGC TACTATATGT CAAGTGCTGT GCAAGGTATT |
| 1921 | ACACTCTGTA ATTGAATATT ATTCCTCAAA AAATTGCACA |
| 1961 | TAGTAGAACG CTATCTGGGA AGCTGTTTTT TTCAGTTTTG |
| 2001 | ATATTTCTAG CTTATCTACT TCCAAACTAA TTTTTGTTTT |
| 2041 | TACTGAGACT AATCTTATTC ATTTTCTCTA ATATGGCAAC |
| 2081 | CATTATAACC TTAATTTATT ATTAACATAC CTAAGAAGTA |
| 2121 | CATTGTTACC TCTATATACC AAAGCACATT TTAAAAGTGC |
| 2161 | CATTAACAAA TGTATCACTA GCCCTCCTTT TTCCAACAAG |
| 2201 | AAGGGACTGA GAGATGCAGA AATATTTGTG ACAAAAAATT |
| 2241 | AAAGCATTTA GGAAAAAAAA AAAAAAAAAA AAAAAAAAAA |
| 2281 | AA |

The protein sequence for this LYVE1 mRNA is available in the NCBI database as accession number AAH26231 (gi: 20070755). See website at ncbi.nlm.nih.gov. This sequence is provided below as follows (SEQ ID NO:4).

| | |
|---|---|
| 1 | MARCFSLVLL LTSIWTTRLL VQGSLRAEEL SIQVSCRIMG |
| 41 | ITLVSKKANQ QLNFTEAKEA CRLLGLSLAG KDQVETALKA |
| 81 | SFETCSYGWV GDGFVVISRI SPNPKCGKNG VGVLIRKVPV |
| 121 | SRQFAAYCYN SSDTWTNSCI PEIITTKDPI FNTQTATQTT |
| 161 | EFIVSDSTYS VASPYSTIPA PTTTPPAPAS TSIPRRKKLI |
| 201 | CVTEVFMETS TMSTETEPFV ENKAAFKNEA AGFGGVPTAL |
| 241 | LVLALLFFGA AAGLGFCYVK RYVKAFPFTN KNQQKEMIET |
| 281 | KVVKEEKAND SNPNEESKKT DKNPEESKSP SKTTVRCLEA |
| 321 | EV |

For example, a TaqMan gene expression (TaqMan) is available from Applied Biosystems that can be used to detect expression of Homo sapiens gene lymphatic vessel endothelial hyaluronan receptor 1 (LYVE1). This expression system was developed for real time qRT-PCR gene expression profiling. Probes available from Applied Biosystems, for example, Pr006486260.1 (Hs00272659_m1), Pr006564284.1 (Hs01119300_g1), Pr006564285.1 (Hs01119301_m1), Pr006564386.1 (Hs01119302_m1) and Pr006564287.1 (Hs01119303_g1), and combinations thereof, can be used in the expression assays to detect LYVE1 expression.

### Allograft Inflammatory Factor (AIF1)

AIF1 is a human gene induced by cytokines and interferon. Its protein product was previously believed to be involved in negative regulation of growth of vascular smooth muscle cells, which contributes to the anti-inflammatory response to vessel wall trauma. Deininger et al., FEBS Lett. 514 (2-3): 115-21. The AIF1 gene expresses three transcripts.

One example of a nucleotide sequence for Homo sapiens allograft inflammatory factor (AIF1) mRNA is available in the NCBI database as accession number NM 032955 (gi: 14574565). See website at ncbi.nlm.nih.gov. This sequence is provided below as follows (SEQ ID NO:5).

| | |
|---|---|
| 1 | CACCTAGCAG TTGGTTGGCA ACCCCTTCCT CAGTCCCCTG |
| 41 | CTGAAAACCC TCCAGTCAGC GCTTATCCCT TCTGCTCTCT |
| 81 | CCCCTCACCC AGAGAAATAC ATGGAGTTTG ACCTTAATGG |
| 121 | AAATGGCGAT ATTGATATCA TGTCCCTGAA ACGAATGCTG |
| 161 | GAGAAACTTG GAGTCCCCAA GACTCACCTA GAGCTAAAGA |
| 201 | AATTAATTGG AGAGGTGTCC AGTGGCTCCG GGGAGACGTT |
| 241 | CAGCTACCCT GACTTTCTCA GGATGATGCT GGGCAAGAGA |
| 281 | TCTGCCATCC TAAAAATGAT CCTGATGTAT GAGGAAAAAG |
| 321 | CGAGAGAAAA GGAAAAGCCA ACAGGCCCCC CAGCCAAGAA |
| 361 | AGCTATCTCT GAGTTGCCCT GATTTGAAGG GAAAAGGGAT |
| 401 | GATGGGATTG AAGGGGCTTC TAATGACCCA GATATGGAAA |
| 441 | CAGAAGACAA AATTGTAAGC CAGAGTCAAC AAATTAAATA |
| 481 | AATTACCCCC TCCTCCAGAT CAA |

The protein sequence for the mRNA is available in the NCBI database as accession number NP 116573 (gi: 14574566). See website at ncbi.nlm.nih.gov. This sequence is provided below as follows (SEQ ID NO:6).

| | |
|---|---|
| 1 | MEFDLNGNGD IDIMSLKRML EKLGVPKTHL ELKKLIGEVS |
| 41 | SGSGETFSYP DFLRMMLGKR SAILKMILMY EEKAREKEKP |
| 81 | TGPPAKKAIS ELP |

Another sequence for Homo sapiens allograft inflammatory factor (AIF1) mRNA is available in the NCBI database as accession number NM001623 (gi: 14574567). See web site at ncbi.nlm.nih.gov. This sequence is provided below as follows (SEQ ID NO:7).

| | |
|---|---|
| 1 | GAGAGAAGGA GAGCCTGCAG ACAGAGGCCT CCAGCTTGGT |
| 41 | CTGTCTCCCC ACCTCTACCA GCATCTGCTG AGCTATGAGC |
| 81 | CAAACCAGGG ATTTACAGGG AGGAAAAGCT TTCGGACTGC |
| 121 | TGAAGGCCCA GCAGGAAGAG AGGCTGGATG AGATCAACAA |
| 161 | GCAATTCCTA GACGATCCCA AATATAGCAG TGATGAGGAT |
| 201 | CTGCCCTCCA AACTGGAAGG CTTCAAAGAG AAATACATGG |
| 241 | AGTTTGACCT TAATGGAAAT GGCGATATTG ATATCATGTC |
| 281 | CCTGAAACGA ATGCTGGAGA AACTTGGAGT CCCCAAGACT |
| 321 | CACCTAGAGC TAAAGAAATT AATTGGAGAG GTGTCCAGTG |
| 361 | GCTCCGGGGA GACGTTCAGC TACCCTGACT TTCTCAGGAT |
| 401 | GATGCTGGGC AAGAGATCTG CCATCCTAAA AATGATCCTG |
| 441 | ATGTATGAGG AAAAAGCGAG AGAAAAGGAA AAGCCAACAG |
| 481 | GCCCCCCAGC CAAGAAAGCT ATCTCTGAGT TGCCCTGATT |
| 521 | TGAAGGGAAA AGGGATGATG GGATTGAAGG GGCTTCTAAT |
| 561 | GACCCAGATA TGGAAACAGA AGACAAAATT GTAAGCCAGA |
| 601 | GTCAACAAAT TAAATAAATT ACCCCCTCCT CCAGATCAA |

The protein sequence for the mRNA is available in the NCBI database as accession number NP 001614 (gi: 14574568). See website at ncbi.nlm.nih.gov. This sequence is provided below as follows (SEQ ID NO:8).

| | |
|---|---|
| 1 | MSQTRDLQGG KAFGLLKAQQ EERLDEINKQ FLDDPKYSSD |
| 41 | EDLPSKLEGF KEKYMEFDLN GNGDIDIMSL KRMLEKLGVP |
| 81 | KTHLELKKLI GEVSSGSGET FSYPDFLRMM LGKRSAILKM |
| 121 | ILMYEEKARE KEKPTGPPAK KAISELP |

In some embodiments, a TaqMan gene expression (TaqMan) from Applied Biosystems can be used to detect expression of Homo sapiens allograft inflammatory factor (AIF1). This expression system was developed for real time qRT-PCR gene expression profiling. Probes and the following probes can be employed in this expression system: Pr006488966.1 (Hs00357551_g1); Pr006605037.1 (Hs00610419_g1); Pr006607042.1 (Hs00741549_g1); Pr006611635.1 (Hs00894884_g1); Pr006611636.1 (Hs00894885_g1); Pr006612982.1 (Hs00897091_g1) and combinations thereof.

### FYN Binding Protein (FYB)

FYN binding protein (FYB) binds to the SH2 domains of FYN and SLP-76 (da Silva A J, Rudd C E. J Biol Chem. 1993; 268:16537-43; da Silva A J, Rosenfield J M, Mueller I, Bouton A, Hirai H, Rudd C E. J Immunol. 1997; 158:2007-16; da Silva A J, Li Z, de Vera C, Canto E, Findell P, Rudd C E. Proc Natl Acad Sci USA. 1997; 94:7493-7498). A similar protein, SLAP (for SLP-associated protein), has been cloned by others (Musci M A, Hendricks-Taylor L R, Motto D G, Paskind M, Kamens J, Turck C W, Koretzky G A. J Biol Chem. 1997; 272:11674-11677). Expression of FYB/SLAP is restricted to T cells, thymocytes, and myeloid cells and does not occur in B cells (da Silva A J, Li Z, de Vera C, Canto E, Findell P, Rudd C E. Proc Natl Acad Sci USA. 1997; 94:7493-7498). It has several proline-rich sequences, multiple tyrosine-based motifs, two stretches of highly charged residues similar to nuclear localization sequences, and an SH3-like domain. FYB/SLAP shows some basal phosphorylation in resting T cells, but it undergoes increased phosphorylation in response to TcR ligation. Consistent with the finding that FYB preferentially associates with FYN, T cells from FYN-negative mice show a marked reduction in FYB phosphorylation. FYB/SLAP has been implicated in IL-2 secretion and in the negative regulation of IL-2 transcription. These observations point to a role for FYB in signaling mediated by SLP-76 and the FYN kinase.

One nucleotide sequence for Homo sapiens FYN binding protein (FYB) mRNA is available in the NCBI database as accession number NM 001465 (gi: 42476117). See web site at ncbi.nlm.nih.gov. This sequence is provided below as follows (SEQ ID NO:9)

| | |
|---|---|
| 1 | CCGCAGTTCT TGAGTTCCAC ATGCAGAGCA GATGCGACAG |
| 41 | CTAGAAGTGA GTAGGGCCCA GACCCTGGCC CAGGAAGATC |
| 81 | CACTAAAGGA GGCCATCCTT CCGCCTTCTT CTGCAGGAGT |
| 121 | CAGGATGGAA AGGCAGATGT AAAGTCCCTC ATGGCGAAAT |
| 161 | ATAACACGGG GGGCAACCCG ACAGAGGATG TCTCAGTCAA |
| 201 | TAGCCGACCC TTCAGAGTCA CAGGGCCAAA CTCATCTTCA |
| 241 | GGAATACAAG CAAGAAAGAA CTTATTCAAC AACCAAGGAA |
| 281 | ATGCCAGCCC TCCTGCAGGA CCCAGCAATG TACCTAAGTT |
| 321 | TGGGTCCCCA AAGCCACCTG TGGCAGTCAA ACCTTCTTCT |
| 361 | GAGGAAAAGC CTGACAAGGA ACCCAAGCCC CCGTTTCTAA |
| 401 | AGCCCACTGG AGCAGGCCAA AGATTCGGAA CACCAGCCAG |
| 441 | CTTGACCACC AGAGACCCCG AGGCGAAAGT GGGATTTCTG |
| 481 | AAACCTGTAG GCCCCAAGCC CATCAACTTG CCCAAAGAAG |
| 521 | ATTCCAAACC TACATTTCCC TGGCCTCCTG GAAACAAGCC |
| 561 | ATCTCTTCAC AGTGTAAACC AAGACCATGA CTTAAAGCCA |
| 601 | CTAGGCCCGA AATCTGGGCC TACTCCTCCA ACCTCAGAAA |
| 641 | ATGAACAGAA GCAAGCGTTT CCCAAATTGA CTGGGGTTAA |
| 681 | AGGGAAATTT ATGTCAGCAT CACAAGATCT TGAACCCAAG |
| 721 | CCCCTCTTCC CCAAACCCGC CTTTGGCCAG AAGCCGCCCC |
| 761 | TAAGTACCGA GAACTCCCAT GAAGACGAAA GCCCCATGAA |
| 801 | GAATGTGTCT TCATCAAAAG GGTCCCCAGC TCCCCTGGGA |
| 841 | GTCAGGTCCA AAAGCGGCCC TTTAAAACCA GCAAGGGAAG |
| 881 | ACTCAGAAAA TAAAGACCAT GCAGGGGAGA TTTCAAGTTT |
| 921 | GCCCTTTCCT GGAGTGGTTT TGAAACCTGC TGCGAGCAGG |
| 961 | GGAGGCCCAG GTCTCTCCAA AAATGGTGAA GAAAAAAAGG |
| 1001 | AAGATAGGAA GATAGATGCT GCTAAGAACA CCTTCCAGAG |
| 1041 | CAAAATAAAT CAGGAAGAGT TGGCCTCAGG GACTCCTCCT |
| 1081 | GCCAGGTTCC CTAAGGCCCC TTCTAAGCTG ACAGTGGGGG |
| 1121 | GGCCATGGGG CCAAAGTCAG GAAAAGGAAA AGGGAGACAA |
| 1161 | GAATTCAGCC ACCCCGAAAC AGAAGCCATT GCCTCCCTTG |
| 1201 | TTTACCTTGG GTCCACCTCC ACCAAAACCC AACAGACCAC |
| 1241 | CAAATGTTGA CCTGACGAAA TTCCACAAAA CCTCTTCTGG |
| 1281 | AAACAGTACT AGCAAAGGCC AGACGTCTTA CTCAACAACT |
| 1321 | TCCCTGCCAC CACCTCCACC ATCCCATCCG GCCAGCCAAC |
| 1361 | CACCATTGCC AGCATCTCAC CCATCACAAC CACCAGTCCC |
| 1401 | AAGCCTACCT CCCAGAAACA TTAAACCTCC GTTTGACCTA |
| 1441 | AAAAGCCCTG TCAATGAAGA CAATCAAGAT GGTGTCACGC |
| 1481 | ACTCTGATGG TGCTGGAAAT CTAGATGAGG AACAAGACAG |
| 1521 | TGAAGGAGAA ACATATGAAG ACATAGAAGC ATCCAAAGAA |
| 1561 | AGAGAGAAGA AAAGGGAAAA GGAAGAAAAG AAGAGGTTAG |
| 1601 | AGCTGGAGAA AAAGGAACAG AAAGAGAAAG AAAAGAAAGA |
| 1641 | ACAAGAAATA AAGAAGAAAT TTAAACTAAC AGGCCCTATT |
| 1681 | CAAGTCATCC ATCTTGCAAA AGCTTGTTGT GATGTCAAAG |
| 1721 | GAGGAAAGAA TGAACTGAGC TTCAAGCAAG GAGAGCAAAT |
| 1761 | TGAAATCATC CGCATCACAG ACAACCCAGA AGGAAAATGG |
| 1801 | TTGGGCAGAA CAGCAAGGGG TTCATATGGC TATATTAAAA |
| 1841 | CAACTGCTGT AGAGATTGAC TATGATTCTT TGAAACTGAA |
| 1881 | AAAAGACTCT CTTGGTGCCC CTTCAAGACC TATTGAAGAT |
| 1921 | GACCAAGAAG TATATGATGA TGTTGCAGAG CAGGATGATA |
| 1961 | TTAGCAGCCA CAGTCAGAGT GGAAGTGGAG GGATATTCCC |
| 2001 | TCCACCACCA GATGATGACA TTTATGATGG GATTGAAGAG |
| 2041 | GAAGATGCTG ATGATGGCTC CACACTACAG GTTCAAGAGA |
| 2081 | AGAGTAATAC GTGGTCCTGG GGGATTTTGA AGATGTTAAA |
| 2121 | GGGAAAAGAT GACAGAAAGA AAAGTATACG AGAGAAACCT |
| 2161 | AAAGTCTCTG ACTCAGACAA TAATGAAGGT TCATCTTTCC |
| 2201 | CTGCTCCTCC TAAACAATTG GACATGGGAG ATGAAGTTTA |
| 2241 | CGATGATGTG GATACCTCTG ATTTCCCTGT TTCATCAGCA |
| 2281 | GAGATGAGTC AAGGAACTAA TGTTGGAAAA GCTAAGACAG |
| 2321 | AAGAAAAGGA CCTTAAGAAG CTAAAAAAGC AGGAAAAAGA |
| 2361 | AGAAAAAGAC TTCAGGAAAA AATTTAAATA TGATGGTGAA |
| 2401 | ATTAGAGTCC TATATTCAAC TAAAGTTACA ACTTCCATAA |
| 2441 | CTTCTAAAAA GTGGGGAACC AGAGATCTAC AGGTAAAACC |
| 2481 | TGGTGAATCT CTAGAAGTTA TACAAACCAC AGATGACACA |
| 2521 | AAAGTTCTCT GCAGAAATGA AGAAGGGAAA TATGGTTATG |
| 2561 | TCCTTCGGAG TTACCTAGCG GACAATGATG GAGAGATCTA |
| 2601 | TGATGATATT GCTGATGGCT GCATCTATGA CAATGACTAG |
| 2641 | CACTCAACTT TGGTCATTCT GCTGTGTTCA TTAGGTGCCA |
| 2681 | ATGTGAAGTC TGGATTTTAA TTGGCATGTT ATTGGGTATC |
| 2721 | AAGAAAATTA ATGCACAAAA CCACTTATTA TCATTTGTTA |
| 2761 | TGAAATCCCA ATTATCTTTA CAAAGTGTTT AAAGTTTGAA |
| 2801 | CATAGAAAAT AATCTCTCTG CTTAATTGTT AACTCAGAAG |
| 2841 | ACTACATTAG TGAGATGTAA GAATTATTAA ATATTCCATT |
| 2881 | TCCGCTTTGG CTACAATTAT GAAGAAGTTG AAGGTACTTC |
| 2921 | TTTTAGACCA CCAGTAAATA ATCCTCCTTC AAAAAATAAA |
| 2961 | AATAAAAGAA AAAGGAAAAT CATTCAGGAA GAAATGACCT |
| 3001 | GTCTAAAAAA ACCTAAGGAA GAATAATAAT ATAAGAAAGG |
| 3041 | AAATTTAAAA ACATTCCACA AGAAGAAAAA TTATTGTTTA |
| 3081 | TACTTCTACT TATGGTTATA TCTTATATTC TCTATTCAAG |
| 3121 | TGACCTGTCT TTTAAAAAGG CAGTGCTGTC TTACCTCTTG |
| 3161 | CTAGTGGGTT AAATGTTTTC AAAAATTATA GCAGTAGTAG |
| 3201 | AAGTTTTGTA TAAAATTTGT CCTTATTTGT TAATTGTATA |
| 3241 | TAAATGTTAA TTATTTGATA CGAATGTTAT GCATTTAGTA |
| 3281 | TGCACATTGA AGTCTAAACT GTAGAAGAGT CTAAAACAAG |
| 3321 | TTCTCTTTTT GCAGATTCAC ATACTAATGG TTTAATTCTG |
| 3361 | TGCTCTGTTT AAAGTACTAT TATAACTAGA GTAGATCTGA |
| 3401 | ATGAGGATAA CCCTAAAATC ATGAGGAATG GAAGAATGGA |
| 3441 | CCTTGAAACT ACCTAGGCTT TTATGCATGG CACCTCTTTA |
| 3481 | TAATGAAGAC ACTTTTTAAA GTTTTTGTTT TTGTTTCAAT |
| 3521 | TACCGCTAGA TTTTTTTTTC TCTTTTTTTA AAATCCATTT |
| 3561 | TACTGGAAAG TTGGCCAGCA GAGGGAGTAG AAATTATTAA |
| 3601 | AATTCTAGTG TTTGGATTGG GCCCTTCTCT AACAGTACAT |
| 3641 | ACTCATTCCC AAAGCAATCC AAAAACAAAA TGTGAACCAT |
| 3681 | TTGGGTTTCA AATGTTAAGA ACACTAAATA GCATGATTTA |
| 3721 | AAAAATGAAA AATGCTAACA CCCAAGAAAA GAAGATATTA |
| 3761 | AGTGCTTTTT AACAACTCCT AGAGTACAAA ATGAGTACAT |
| 3801 | CATAATGCTG GCTCTTCTAC TAATGAACCA TCGAGTGATA |
| 3841 | TTGAATAAAT TATTTATCTT CTCAGTTTCC TTATCTGTAA |
| 3881 | ATTACAATAT TAGACTAAGT AAGTTTTTCC AACTCTTCAC |
| 3921 | TACCAATTAC CTTAGGCTTT TATAATGCTC CGCCTACTTC |
| 3961 | AGTCCCATGT TTCAGAAGCT TTTGTCTATT TTTTAAACTC |
| 4001 | ATTGATTAAA TAATGATTAA TGCATTCTCC ACATTTTAAT |
| 4041 | ATTGCAAAGG CCCATTGGAG TTTCTGAAGT GGCTCCACAG |
| 4081 | AATTGAAATA ATTTCAAATA ACTGTAAAGG AACTGAAAAT |
| 4121 | CTTCACAGAG ATGAAGTGGG GTTTCCATTA GGTGCTTTGA |
| 4161 | AATTTGATAA CAAATCATCA ACTTCCACTG GTCAATATAT |
| 4201 | AGATTTTGGG TGTCTGAGGC CCCAAGATTA GATGCCACTA |
| 4241 | ATCTCCAAAG ATTCCCTCCA ATTATGAAAT ATTTTAATGT |
| 4281 | CTACTTTTAG AGAGCACTAG CCAGTATATG ACCATGTGAT |
| 4321 | TAATTTCTTT TCACACTAGA TAAAATTACC TGGTTCAAAA |
| 4361 | GTGGTTTTTG TTTATTAAAT TTGGTAATAA ATATATATAA |
| 4401 | TACACAGACA GGATAGTTTT TATGCTGAAG TTTTTGGCCA |
| 4441 | GCTTTAGTTT GAGGACTCCT TGATAAGCTT GCTAAACTTT |
| 4481 | CAGAGTGCCC TGAGACACTT CCAGCCATCC CTCCTCCTGC |
| 4521 | CTTCATTGGG GCAGACTTGC ATTGCAGTCT GACAGTAATT |
| 4561 | TTTTTTCTGA TTGAGAATTA TGTAAATTCA ATACAATGTC |
| 4601 | AGTTTTTAAA AGTCAAAGTT AGATCAAGAG AATATTTCAG |
| 4641 | AGTTTTGGTT TACACATCAA GAAACAGACA CACATACCTA |
| 4681 | GGAAAGATTT ACACAATAGA TAATCATCTT AATGTGAAAG |
| 4721 | ATATTTGAAG TATTAATTTT AATATATTAA ATATGATTTC |
| 4761 | TGTTATAGTC TTCTGTATGG AATTTTGTCA CTTAAGATGA |
| 4801 | GCTGCAAATA AATAATACCT TCAATGGAAA AAAAAAAAAA |
| 4841 | AAAAAAAAAA AAAAAAAAAA AAAAAAAAAA AAAAAA |

The protein sequence for the mRNA is available in the NCBI database as accession number NP 001465 (gi: 42476118). See website at ncbi.nlm.nih.gov. This sequence is provided below as follows (SEQ ID NO:10).

| | |
|---|---|
| 1 | MAKYNTGGNP TEDVSVNSRP FRVTGPNSSS GIQARKNLFN |
| 41 | NQGNASPPAG PSNVPKFGSP KPPVAVKPSS EEKPDKEPKP |
| 81 | PFLKPTGAGQ RFGTPASLTT RDPEAKVGFL KPVGPKPINL |
| 121 | PKEDSKPTFP WPPGNKPSLH SVNQDHDLKP LGPKSGPTPP |
| 161 | TSENEQKQAF PKLTGVKGKF MSASQDLEPK PLFPKPAFGQ |
| 201 | KPPLSTENSH EDESPMKNVS SSKGSPAPLG VRSKSGPLKP |
| 241 | AREDSENKDH AGEISSLPFP GVVLKPAASR GGPGLSKNGE |
| 281 | EKKEDRKIDA AKNTFQSKIN QEELASGTPP ARFPKAPSKL |
| 321 | TVGGPWGQSQ EKEKGDKNSA TPKQKPLPPL FTLGPPPPKP |
| 361 | NRPPNVDLTK FHKTSSGNST SKGQTSYSTT SLPPPPPSHP |
| 401 | ASQPPLPASH PSQPPVPSLP PRNIKPPFDL KSPVNEDNQD |
| 441 | GVTHSDGAGN LDEEQDSEGE TYEDIEASKE REKKREKEEK |
| 481 | KRLELEKKEQ KEKEKKEQEI KKKFKLTGPI QVIHLAKACC |
| 521 | DVKGGKNELS FKQGEQIEII RITDNPEGKW LGRTARGSYG |
| 561 | YIKTTAVEID YDSLKLKKDS LGAPSRPIED DQEVYDDVAE |
| 601 | QDDISSHSQS GSGGIFPPPP DDDIYDGIEE EDADDGSTLQ |
| 641 | VQEKSNTWSW GILKMLKGKD DRKKSIREKP KVSDSDNNEG |
| 681 | SSFPAPPKQL DMGDEVYDDV DTSDFPVSSA EMSQGTNVGK |
| 721 | AKTEEKDLKK LKKQEKEEKD FRKKFKYDGE IRVLYSTKVT |
| 761 | TSITSKKWGT RDLQVKPGES LEVIQTTDDT KVLCRNEEGK |
| 801 | YGYVLRSYLA DNDGEIYDDI ADGCIYDND |

A related nucleotide sequence for Homo sapiens FYN binding protein (FYB) mRNA is available in the NCBI database as accession number NM 199335 (gi: 42476114). See web site at ncbi.nlm.nih.gov. The sequence of this FYB cDNA is provided below (SEQ ID NO:11).

| | |
|---|---|
| 1 | CCGCAGTTCT TGAGTTCCAC ATGCAGAGCA GATGCGACAG |
| 41 | CTAGAAGTGA GTAGGGCCCA GACCCTGGCC CAGGAAGATC |
| 81 | CACTAAAGGA GGCCATCCTT CCGCCTTCTT CTGCAGGAGT |
| 121 | CAGGATGGAA AGGCAGATGT AAAGTCCCTC ATGGCGAAAT |
| 161 | ATAACACGGG GGGCAACCCG ACAGAGGATG TCTCAGTCAA |
| 201 | TAGCCGACCC TTCAGAGTCA CAGGGCCAAA CTCATCTTCA |
| 241 | GGAATACAAG CAAGAAAGAA CTTATTCAAC AACCAAGGAA |
| 281 | ATGCCAGCCC TCCTGCAGGA CCCAGCAATG TACCTAAGTT |
| 321 | TGGGTCCCCA AAGCCACCTG TGGCAGTCAA ACCTTCTTCT |
| 361 | GAGGAAAAGC CTGACAAGGA ACCCAAGCCC CCGTTTCTAA |
| 401 | AGCCCACTGG AGCAGGCCAA AGATTCGGAA CACCAGCCAG |
| 441 | CTTGACCACC AGAGACCCCG AGGCGAAAGT GGGATTTCTG |
| 481 | AAACCTGTAG GCCCCAAGCC CATCAACTTG CCCAAAGAAG |
| 521 | ATTCCAAACC TACATTTCCC TGGCCTCCTG GAAACAAGCC |
| 561 | ATCTCTTCAC AGTGTAAACC AAGACCATGA CTTAAAGCCA |
| 601 | CTAGGCCCGA AATCTGGGCC TACTCCTCCA ACCTCAGAAA |
| 641 | ATGAACAGAA GCAAGCGTTT CCCAAATTGA CTGGGGTTAA |
| 681 | AGGGAAATTT ATGTCAGCAT CACAAGATCT TGAACCCAAG |
| 721 | CCCCTCTTCC CCAAACCCGC CTTTGGCCAG AAGCCGCCCC |
| 761 | TAAGTACCGA GAACTCCCAT GAAGACGAAA GCCCCATGAA |
| 801 | GAATGTGTCT TCATCAAAAG GGTCCCCAGC TCCCCTGGGA |
| 841 | GTCAGGTCCA AAAGCGGCCC TTTAAAACCA GCAAGGGAAG |
| 881 | ACTCAGAAAA TAAAGACCAT GCAGGGGAGA TTTCAAGTTT |
| 921 | GCCCTTTCCT GGAGTGGTTT TGAAACCTGC TGCGAGCAGG |
| 961 | GGAGGCCCAG GTCTCTCCAA AAATGGTGAA GAAAAAAAGG |
| 1001 | AAGATAGGAA GATAGATGCT GCTAAGAACA CCTTCCAGAG |
| 1041 | CAAAATAAAT CAGGAAGAGT TGGCCTCAGG GACTCCTCCT |
| 1081 | GCCAGGTTCC CTAAGGCCCC TTCTAAGCTG ACAGTGGGGG |
| 1121 | GGCCATGGGG CCAAAGTCAG GAAAAGGAAA AGGGAGACAA |
| 1161 | GAATTCAGCC ACCCCGAAAC AGAAGCCATT GCCTCCCTTG |
| 1201 | TTTACCTTGG GTCCACCTCC ACCAAAACCC AACAGACCAC |
| 1241 | CAAATGTTGA CCTGACGAAA TTCCACAAAA CCTCTTCTGG |
| 1281 | AAACAGTACT AGCAAAGGCC AGACGTCTTA CTCAACAACT |
| 1321 | TCCCTGCCAC CACCTCCACC ATCCCATCCG GCCAGCCAAC |
| 1361 | CACCATTGCC AGCATCTCAC CCATCACAAC CACCAGTCCC |
| 1401 | AAGCCTACCT CCCAGAAACA TTAAACCTCC GTTTGACCTA |
| 1441 | AAAAGCCCTG TCAATGAAGA CAATCAAGAT GGTGTCACGC |
| 1481 | ACTCTGATGG TGCTGGAAAT CTAGATGAGG AACAAGACAG |
| 1521 | TGAAGGAGAA ACATATGAAG ACATAGAAGC ATCCAAAGAA |
| 1561 | AGAGAGAAGA AAAGGGAAAA GGAAGAAAAG AAGAGGTTAG |
| 1601 | AGCTGGAGAA AAAGGAACAG AAAGAGAAAG AAAAGAAAGA |
| 1641 | ACAAGAAATA AAGAAGAAAT TTAAACTAAC AGGCCCTATT |
| 1681 | CAAGTCATCC ATCTTGCAAA AGCTTGTTGT GATGTCAAAG |
| 1721 | GAGGAAAGAA TGAACTGAGC TTCAAGCAAG GAGAGCAAAT |
| 1761 | TGAAATCATC CGCATCACAG ACAACCCAGA AGGAAAATGG |
| 1801 | TTGGGCAGAA CAGCAAGGGG TTCATATGGC TATATTAAAA |
| 1841 | CAACTGCTGT AGAGATTGAC TATGATTCTT TGAAACTGAA |
| 1881 | AAAAGACTCT CTTGGTGCCC CTTCAAGACC TATTGAAGAT |
| 1921 | GACCAAGAAG TATATGATGA TGTTGCAGAG CAGGATGATA |
| 1961 | TTAGCAGCCA CAGTCAGAGT GGAAGTGGAG GGATATTCCC |
| 2001 | TCCACCACCA GATGATGACA TTTATGATGG GATTGAAGAG |
| 2041 | GAAGATGCTG ATGATGGTTT CCCTGCTCCT CCTAAACAAT |
| 2081 | TGGACATGGG AGATGAAGTT TACGATGATG TGGATACCTC |
| 2121 | TGATTTCCCT GTTTCATCAG CAGAGATGAG TCAAGGAACT |
| 2161 | AATGTTGGAA AAGCTAAGAC AGAAGAAAAG GACCTTAAGA |
| 2201 | AGCTAAAAAA GCAGGAAAAA GAAGAAAAAG ACTTCAGGAA |
| 2241 | AAAATTTAAA TATGATGGTG AAATTAGAGT CCTATATTCA |
| 2281 | ACTAAAGTTA CAACTTCCAT AACTTCTAAA AAGTGGGGAA |
| 2321 | CCAGAGATCT ACAGGTAAAA CCTGGTGAAT CTCTAGAAGT |
| 2361 | TATACAAACC ACAGATGACA CAAAAGTTCT CTGCAGAAAT |
| 2401 | GAAGAAGGGA AATATGGTTA TGTCCTTCGG AGTTACCTAG |
| 2441 | CGGACAATGA TGGAGAGATC TATGATGATA TTGCTGATGG |
| 2481 | CTGCATCTAT GACAATGACT AGCACTCAAC TTTGGTCATT |
| 2521 | CTGCTGTGTT CATTAGGTGC CAATGTGAAG TCTGGATTTT |
| 2561 | AATTGGCATG TTATTGGGTA TCAAGAAAAT TAATGCACAA |
| 2601 | AACCACTTAT TATCATTTGT TATGAAATCC CAATTATCTT |
| 2641 | TACAAAGTGT TTAAAGTTTG AACATAGAAA ATAATCTCTC |
| 2681 | TGCTTAATTG TTAACTCAGA AGACTACATT AGTGAGATGT |
| 2721 | AAGAATTATT AAATATTCCA TTTCCGCTTT GGCTACAATT |
| 2761 | ATGAAGAAGT TGAAGGTACT TCTTTTAGAC CACCAGTAAA |
| 2801 | TAATCCTCCT TCAAAAAATA AAAATAAAAG AAAAAGGAAA |
| 2841 | ATCATTCAGG AAGAAATGAC CTGTCTAAAA AAACCTAAGG |
| 2881 | AAGAATAATA ATATAAGAAA GGAAATTTAA AAACATTCCA |
| 2921 | CAAGAAGAAA AATTATTGTT TATACTTCTA CTTATGGTTA |
| 2961 | TATCTTATAT TCTCTATTCA AGTGACCTGT CTTTTAAAAA |
| 3001 | GGCAGTGCTG TCTTACCTCT TGCTAGTGGG TTAAATGTTT |
| 3041 | TCAAAAATTA TAGCAGTAGT AGAAGTTTTG TATAAAATTT |
| 3081 | GTCCTTATTT GTTAATTGTA TATAAATGTT AATTATTTGA |
| 3121 | TACGAATGTT ATGCATTTAG TATGCACATT GAAGTCTAAA |
| 3161 | CTGTAGAAGA GTCTAAAACA AGTTCTCTTT TTGCAGATTC |
| 3201 | ACATACTAAT GGTTTAATTC TGTGCTCTGT TTAAAGTACT |
| 3241 | ATTATAACTA GAGTAGATCT GAATGAGGAT AACCCTAAAA |
| 3281 | TCATGAGGAA TGGAAGAATG GACCTTGAAA CTACCTAGGC |
| 3321 | TTTTATGCAT GGCACCTCTT TATAATGAAG ACACTTTTTA |
| 3361 | AAGTTTTTGT TTTTGTTTCA ATTACCGCTA GATTTTTTTT |
| 3401 | TCTCTTTTTT TAAAATCCAT TTTACTGGAA AGTTGGCCAG |
| 3441 | CAGAGGGAGT AGAAATTATT AAAATTCTAG TGTTTGGATT |
| 3481 | GGGCCCTTCT CTAACAGTAC ATACTCATTC CCAAAGCAAT |
| 3521 | CCAAAAACAA AATGTGAACC ATTTGGGTTT CAAATGTTAA |
| 3561 | GAACACTAAA TAGCATGATT TAAAAAATGA AAAATGCTAA |
| 3601 | CACCCAAGAA AAGAAGATAT TAAGTGCTTT TTAACAACTC |
| 3641 | CTAGAGTACA AAATGAGTAC ATCATAATGC TGGCTCTTCT |
| 3681 | ACTAATGAAC CATCGAGTGA TATTGAATAA ATTATTTATC |
| 3721 | TTCTCAGTTT CCTTATCTGT AAATTACAAT ATTAGACTAA |
| 3761 | GTAAGTTTTT CCAACTCTTC ACTACCAATT ACCTTAGGCT |
| 3801 | TTTATAATGC TCCGCCTACT TCAGTCCCAT GTTTCAGAAG |
| 3841 | CTTTTGTCTA TTTTTTAAAC TCATTGATTA AATAATGATT |
| 3881 | AATGCATTCT CCACATTTTA ATATTGCAAA GGCCCATTGG |
| 3921 | AGTTTCTGAA GTGGCTCCAC AGAATTGAAA TAATTTCAAA |
| 3961 | TAACTGTAAA GGAACTGAAA ATCTTCACAG AGATGAAGTG |
| 4001 | GGGTTTCCAT TAGGTGCTTT GAAATTTGAT AACAAATCAT |
| 4041 | CAACTTCCAC TGGTCAATAT ATAGATTTTG GGTGTCTGAG |
| 4081 | GCCCCAAGAT TAGATGCCAC TAATCTCCAA AGATTCCCTC |
| 4121 | CAATTATGAA ATATTTTAAT GTCTACTTTT AGAGAGCACT |
| 4161 | AGCCAGTATA TGACCATGTG ATTAATTTCT TTTCACACTA |
| 4201 | GATAAAATTA CCTGGTTCAA AAGTGGTTTT TGTTTATTAA |
| 4241 | ATTTGGTAAT AAATATATAT AATACACAGA CAGGATAGTT |
| 4281 | TTTATGCTGA AGTTTTTGGC CAGCTTTAGT TTGAGGACTC |
| 4321 | CTTGATAAGC TTGCTAAACT TTCAGAGTGC CCTGAGACAC |
| 4361 | TTCCAGCCAT CCCTCCTCCT GCCTTCATTG GGGCAGACTT |
| 4401 | GCATTGCAGT CTGACAGTAA TTTTTTTTCT GATTGAGAAT |
| 4441 | TATGTAAATT CAATACAATG TCAGTTTTTA AAAGTCAAAG |
| 4481 | TTAGATCAAG AGAATATTTC AGAGTTTTGG TTTACACATC |
| 4521 | AAGAAACAGA CACACATACC TAGGAAAGAT TTACACAATA |
| 4561 | GATAATCATC TTAATGTGAA AGATATTTGA AGTATTAATT |
| 4601 | TTAATATATT AAATATGATT TCTGTTATAG TCTTCTGTAT |
| 4641 | GGAATTTTGT CACTTAAGAT GAGCTGCAAA TAAATAATAC |
| 4681 | CTTCAATGGA AAAAAAAAAA AAAAAAAAAA AAAAAAAAAA |
| 4721 | AAAAAAAAAA AAAAAAAA |

In some embodiments, a TaqMan gene expression (TaqMan) from Applied Biosystems can be used to detect expression of Homo sapiens FYN binding protein (FYN). This expression system was developed for real time qRT-PCR gene expression profiling. Probes and the following probes can be employed in this expression system: Pr006477714.1 (Hs00175177_m1); Pr006529920.1 (Hs01061556_m1); Pr006529921.1 (Hs01061557_m1); Pr006529922.1 (Hs01061558_g1); Pr006529923.1 (Hs01061559_m1); Pr006529924.1 (Hs01061560_m1); Pr006529925.1 (Hs01061561_m1); Pr006529928.1 (Hs01061565_m1); Pr006529929.1 (Hs01061569_g1); Pr006532524.1 (Hs01065871_m1); Pr006532525.1 (Hs01065872_m1); Pr006532526.1 (Hs01065874_m1); Pr006534737.1 (Hs01069968_m1) and combinations thereof.

### Myeloid/Lymphoid or Mixed-Lineage Leukemia, Translocated to, 3 (MLLT3)

One nucleotide sequence for Homo sapiens myeloid/lymphoid or mixed-lineage leukemia, translocated to, 3 (MLLT3) mRNA is available in the NCBI database as accession number NM 004529 (gi: 156104888). See website at ncbi.nlm.nih.gov. This sequence is provided below as follows (SEQ ID NO:12).

| | |
|---|---|
| 1 | ACGGCGCATG CTCCGCAATC ATCTTCTTTA CCCTGGAGCT |
| 41 | GCTGCTGCTG CTGCTGCTTT TGCTTTTGGG GCTGAGTTTA |
| 81 | ATAAGCGAGC GAGCGAGCAA GCGAGCGCGG GGGGAAAAAG |
| 121 | GCAGAGAATG TCCGCCATCT ACCCTCCGCT CCTGGGCGCG |
| 161 | CTCTCATTCA TAGCAGCCTC TTCATGAATT ACAGCTGAGG |
| 201 | GGGGGCGGAG GAGGGGGGGG TACCACACAA CACCCCAGCA |
| 241 | AACCTCCGGG CCCCCAGGCA TGGCTAGCTC GTGTGCCGTG |
| 281 | CAGGTGAAGC TGGAGCTGGG GCACCGCGCC CAGGTGAGGA |
| 321 | AAAAACCCAC CGTGGAGGGC TTCACCCACG ACTGGATGGT |
| 361 | GTTCGTACGC GGTCCGGAGC ACAGTAACAT ACAGCACTTT |
| 401 | GTGGAGAAAG TCGTCTTCCA CTTGCACGAA AGCTTTCCTA |
| 441 | GGCCAAAAAG AGTGTGCAAA GATCCACCTT ACAAAGTAGA |
| 481 | AGAATCTGGG TATGCTGGTT TCATTTTGCC AATTGAAGTT |
| 521 | TATTTTAAAA ACAAGGAAGA ACCTAGGAAA GTCCGCTTTG |
| 561 | ATTATGACTT ATTCCTGCAT CTTGAAGGCC ATCCACCAGT |
| 601 | GAATCACCTC CGCTGTGAAA AGCTAACTTT CAACAACCCC |
| 641 | ACAGAGGACT TTAGGAGAAA GTTGCTGAAG GCAGGAGGGG |
| 681 | ACCCTAATAG GAGTATTCAT ACCAGCAGCA GCAGCAGCAG |
| 721 | CAGCAGTAGC AGCAGCAGCA GCAGCAGCAG CAGCAGCAGT |
| 761 | AGCAGCAGCA GCAGCAGCAG CAGCAGCAGC AGTAGCAGCA |
| 801 | GCAGTAGCAG CAGCAGCAGC AGCAGTAGTA CCAGTTTTTC |
| 841 | AAAGCCTCAC AAATTAATGA AGGAGCACAA GGAAAAACCT |
| 881 | TCTAAAGACT CCAGAGAACA TAAAAGTGCC TTCAAAGAAC |
| 921 | CTTCCAGGGA TCACAACAAA TCTTCCAAAG AATCCTCTAA |
| 961 | GAAACCCAAA GAAAATAAAC CACTGAAAGA AGAGAAAATA |
| 1001 | GTTCCTAAGA TGGCCTTCAA GGAACCTAAA CCCATGTCAA |
| 1041 | AAGAGCCAAA ACCAGATAGT AACTTACTCA CCATCACCAG |
| 1081 | TGGACAAGAT AAGAAGGCTC CTAGTAAAAG GCCGCCCATT |
| 1121 | TCAGATTCTG AAGAACTCTC AGCCAAAAAA AGGAAAAAGA |
| 1161 | GTAGCTCAGA GGCTTTATTT AAAAGTTTTT CTAGCGCACC |
| 1201 | ACCACTGATA CTCACTTGTT CTGCTGACAA AAAACAGATA |
| 1241 | AAAGATAAAT CTCATGTCAA GATGGGAAAG GTCAAAATTG |
| 1281 | AAAGTGAGAC ATCAGAGAAG AAGAAATCAA CGTTACCGCC |
| 1321 | ATTTGATGAT ATTGTGGATC CCAATGATTC AGATGTGGAG |
| 1361 | GAGAATATAT CCTCTAAATC TGATTCTGAA CAACCCAGTC |
| 1401 | CTGCCAGCTC CAGCTCCAGC TCCAGCTCCA GCTTCACACC |
| 1441 | ATCCCAGACC AGGCAACAAG GTCCTTTGAG GTCTATAATG |
| 1481 | AAAGATCTGC ATTCTGATGA CAATGAGGAG GAATCAGATG |
| 1521 | AAGTGGAGGA TAACGACAAT GACTCTGAAA TGGAGAGGCC |
| 1561 | TGTAAATAGA GGAGGCAGCC GAAGTCGCAG AGTTAGCTTA |
| 1601 | AGTGATGGCA GCGATAGTGA AAGCAGTTCT GCTTCTTCAC |
| 1641 | CCCTACATCA CGAACCTCCA CCACCCTTAC TAAAAACCAA |
| 1681 | CAACAACCAG ATTCTTGAAG TGAAAAGTCC AATAAAGCAA |
| 1721 | AGCAAATCAG ATAAGCAAAT AAAGAATGGT GAATGTGACA |
| 1761 | AGGCATACCT AGATGAACTG GTAGAGCTTC ACAGAAGGTT |
| 1801 | AATGACATTG AGAGAAAGAC ACATTCTGCA GCAGATCGTG |
| 1841 | AACCTTATAG AAGAAACTGG ACACTTTCAT ATCACAAACA |
| 1881 | CAACATTTGA TTTTGATCTT TGCTCGCTGG ACAAAACCAC |
| 1921 | AGTCCGTAAA CTACAGAGTT ACCTGGAAAC ATCTGGAACA |
| 1961 | TCCTGAGGAT ATAACAACTG GATGCATCAA GAACTATTGT |
| 2001 | GTTTTTTTTT TTTGGTTTTT TTTTTTTTTG GTTGTGATTT |
| 2041 | TTTGTTCTTG TTGTTTATAT GAAAACACTC AAAATGATGC |
| 2081 | AACCAAAAGG GAAAAAATAA AAATCAAACA ACCTTCAGCT |
| 2121 | TTATTTTTCT TTAAAGCCAG TCATCATCTC TTGATAAAGG |
| 2161 | AGAGGTTAAA GCAAACCAGC CTCAGCGGAC CACTCTTCTC |
| 2201 | TCCAAGGAAA TCCCCGGGAA GAGTTAGCCT GGATAGCCTT |
| 2241 | GAAAACAAAC AAATCAAACA CAACACAAGA AAACTCAAAG |
| 2281 | AATGTGTATG GTATCATGTA TCTCTCTGTG GTGGTTCATT |
| 2321 | CCACAGGACG AATGCATATT CAACACACTG CCTTATTACA |
| 2361 | TAACTGATCT ATTTATTATC GCATACAGAT ATTCTAAGTC |
| 2401 | GTTGAGGGAA TGACACCATC AGACATTATA AGTACTTGGT |
| 2441 | CCCGTGGATG CTCTTTCAAT GCAGCACCCT TGCCATCCCA |
| 2481 | AGCCCAGTGA CCTTACTCGT ATACCGTGCC ACTTTCCACC |
| 2521 | AACTTTTTCC AAGTCCTTTA ACTCGTTGCA GTCTGTATTT |
| 2561 | TCCACCTTTT GTTTTTCCAG TTCCAGGACA CAGATTATCA |
| 2601 | ACTGGGGGGA CCAAATAGCC ACCTTGATTT TCTTCTTTGT |
| 2641 | GGTCTTTTTC CTGAAAGTTG GGGCCCAGTC CTTGGCTGTA |
| 2681 | TCCATGTAAT GATCTTGGAC CATGGTAGAA AATGCACCAA |
| 2721 | ATAGGATCAT ATGAATTGCT GTCTAGCCTT AGTCAATAAA |
| 2761 | CTTGTAGGAC TTTTAAACAA AAGTGTACCT GTAAATGTCC |
| 2801 | TGAATCCAGC ATTGTTGAGC TGTCATCAAC ATTCTTGTGT |
| 2841 | CTGTTTTACT GTTACAATAT TAGGTGAATA TGGAAGTAAA |
| 2881 | GGCATTCCAC AGGATCATCA TTTAAAAAAA AAGAATTCTG |
| 2921 | GTCCTGTTTT CTAAAAAAAA AAACTGTTGT AGAAATTCTT |
| 2961 | AATTTGGATC TATTTATTAG TCAGAGTTTC AGCTTTCTTC |
| 3001 | AGCTGCCAGT GTGTTACTCA TCTTTATCCT AAAAATCTGG |
| 3041 | AATCAGAGAT TTTTGTTTGT TCACATATGA TTCTCTTAGA |
| 3081 | CACTTTTATA TTTGAAAAAA TTAAAATCTT TCTTTGGGGA |
| 3121 | AAAATTCTTG GTTATTCTGC CATAACAGAT TATGTATTAA |
| 3161 | CTTGTAGATT CAGTGGTTCA ATACCTGTTT AGTTGCTTGC |
| 3201 | TAATATTTCC AGAAGGATTT CTTGTATTGG TGAAAGACGG |
| 3241 | TTGGGGATGG GGGGATTTTT TTGTTCTTGT TGTACCCTTG |
| 3281 | TTTTGAAACT AGAAATCTGT CCTGTGGCAT GCAAAAGAAA |
| 3321 | GCAAATTATT TTTAAAAGAA AAAAACCAAA GTACTTTTGG |
| 3361 | TGTCATTATT CCATCTTCTC CATAAGTGGA GAAATGAAAA |
| 3401 | GTAAGAACAG CTCATCTTCA AAGTTTTTAC TAGAAATTC |

The corresponding protein sequence for this MLLT3 mRNA is available in the NCBI database as accession number NP 004520 (gi: 156104889). See website at ncbi.nlm.nih.gov. This sequence is provided below (SEQ ID NO:13).

| | |
|---|---|
| 1 | MASSCAVQVK LELGHRAQVR KKPTVEGFTH DWMVFVRGPE |
| 41 | HSNIQHFVEK VVFHLHESFP RPKRVCKDPP YKVEESGYAG |
| 81 | FILPIEVYFK NKEEPRKVRF DYDLFLHLEG HPPVNHLRCE |
| 121 | KLTFNNPTED FRRKLLKAGG DPNRSIHTSS SSSSSSSSSS |
| 161 | SSSSSSSSSS SSSSSSSSSS SSSSSSSSSS TSFSKPHKLM |
| 201 | KEHKEKPSKD SREHKSAFKE PSRDHNKSSK ESSKKPKENK |
| 241 | PLKEEKIVPK MAFKEPKPMS KEPKPDSNLL TITSGQDKKA |
| 281 | PSKRPPISDS EELSAKKRKK SSSEALFKSF SSAPPLILTC |
| 321 | SADKKQIKDK SHVKMGKVKI ESETSEKKKS TLPPFDDIVD |
| 361 | PNDSDVEENI SSKSDSEQPS PASSSSSSSS SFTPSQTRQQ |
| 401 | GPLRSIMKDL HSDDNEEESD EVEDNDNDSE MERPVNRGGS |
| 441 | RSRRVSLSDG SDSESSSASS PLHHEPPPPL LKTNNNQILE |
| 481 | VKSPIKQSKS DKQIKNGECD KAYLDELVEL HRRLMTLRER |
| 521 | HILQQIVNLI EETGHFHITN TTFDFDLCSL DKTTVRKLQS |
| 561 | YLETSGTS |

Taqman gene expression systems are also available from Applied Biosystems to detect expression of Homo sapiens myeloid/lymphoid or mixed-lineage leukemia, translocated to, 3 (MLLT3) mRNA. Probes and the following probes can be employed in this expression system: Pr006478130.1 (Hs00180312_m1); Pr006662699.1 (Hs00971090_m1); Pr006662700.1 (Hs00971091_m1); Pr006662701.1 (Hs00971092_m1); Pr006662702.1 (Hs00971093_m1); Pr006662703.1 (Hs00971095_m1); Pr006662704.1 (Hs00971096_m1); Pr006662705.1 (Hs00971097_m1); Pr006662706.1 (Hs00971099_m1) and combinations thereof.

### Purinergic Receptor P2Y, G-Protein Coupled, 1 (P2RY1)

P2Y₁ receptors play a role in calcium signaling in various neurons and glial cells (see, e.g., Fam et al. J. Neurosci. 2003; 23:4437-4444; Fam et al., J. Neurosci. 2000; 20:2800-2808; Saitow et al., J. Neurosci. 2005; 25:2108-2116; and Weissman et al., Neuron. 2004; 43:647-661. Moreover, signaling through recombinant P2Y₁ receptors stimulates mitogen-activated (MAP) kinases (Sellers et al. J. Biol. Chem. 2001; 276:16379-16390). In native cells, P2Y₁ receptors have been implicated to play a role in extracellular signal related kinases (ERK) activation and stretch induced injury in astrocytes (Neary et al. J. Neurosci. 2003; 23:2348-2356).

One nucleotide sequence for Homo sapiens purinergic receptor P2Y, G-protein coupled, 1(P2RY1) mRNA is available in the NCBI database as accession number NM 002563 (gi: 28872741). See website at ncbi.nlm.nih.gov. This sequence is provided below as follows (SEQ ID NO:14).

| | |
|---|---|
| 1 | TCGGCGGAGA CCTGCTCCCC AGAAGACGCC TCCTGCTTCC |
| 41 | CACTGCGCCC TGGAGGACGC GGGCTGGCTG CTGGGCGAGC |
| 81 | TCGGCGGAGG CACGCCCCTC GCCTCCCCGC GGAGTGCGGA |
| 121 | CTCGCCCCGG TGCCCAAACT CCGCCCACCC TCTAGGGAGC |
| 161 | TCCGCTCTCC CGCCTAACCC CGGCACTCCG GACAGAGCTG |
| 201 | GGCCTGGGGA AGGGGTTCCT GAACTACGCG GACGCCGAAC |
| 241 | GGGACGCGCT GCAGAAGCGC ACGAGTCTGC GGCCACGCGC |
| 281 | GCTCCGATGG CTGCCAGGAG CTGAGCTCAG GGTGGGCGGA |
| 321 | GGAAGCGGTT AGACGCCCCG AAACTGAGCT GCACGTTTCT |
| 361 | AAGGTAGGGA GGAGGAAGAT GCCCCCAATT AAGTTGATCT |
| 401 | TTGAGCCAAG GAGGCTGGGG AGCAGCCTCC CCAAGCTAGA |
| 441 | GCCCTGCAGA GCGAGTTTCC CTTGACCTCG CTGCGCCTCT |
| 481 | GGCGCGCTCT GCAGCGCGGA CCCGCGGCCC CTCGGGAAAG |
| 521 | CGCAGTCGGA AAGTTATCCG CGGCGGTTCC CTGCGCGCCC |
| 561 | TGTTGTGTAA GCTCGGCGTT GCCAGCGGAC GGAGAAGTTG |
| 601 | CTGGCTTGCC CGATAGCCCA GTTCGGTGGC GGCCCGGGGC |
| 641 | GGATTTCATG GCCCGCGGCG AACGCGGGGC CAGAGCTGGC |
| 681 | GTGGGCGAGC CCCTGCGCGC CCCCTCCCGC GGGGATCCAG |
| 721 | TTCGCCTGCT CCCTTCCGCT CGCTGGCTTT TCCGATGCTT |
| 761 | GCTGCGCCCC TGGCCGCCGC TGCCCTCTCG CCGCCTCCTA |
| 801 | CCCCTCGGAG CCGCCGCCTA AGTCGAGGAG GAGAGAATGA |
| 841 | CCGAGGTGCT GTGGCCGGCT GTCCCCAACG GGACGGACGC |
| 881 | TGCCTTCCTG GCCGGTCCGG GTTCGTCCTG GGGGAACAGC |
| 921 | ACGGTCGCCT CCACTGCCGC CGTCTCCTCG TCGTTCAAAT |
| 961 | GCGCCTTGAC CAAGACGGGC TTCCAGTTTT ACTACCTGCC |
| 1001 | GGCTGTCTAC ATCTTGGTAT TCATCATCGG CTTCCTGGGC |
| 1041 | AACAGCGTGG CCATCTGGAT GTTCGTCTTC CACATGAAGC |
| 1081 | CCTGGAGCGG CATCTCCGTG TACATGTTCA ATTTGGCTCT |
| 1121 | GGCCGACTTC TTGTACGTGC TGACTCTGCC AGCCCTGATC |
| 1161 | TTCTACTACT TCAATAAAAC AGACTGGATC TTCGGGGATG |
| 1201 | CCATGTGTAA ACTGCAGAGG TTCATCTTTC ATGTGAACCT |
| 1241 | CTATGGCAGC ATCTTGTTTC TGACATGCAT CAGTGCCCAC |
| 1281 | CGGTACAGCG GTGTGGTGTA CCCCCTCAAG TCCCTGGGCC |
| 1321 | GGCTCAAAAA GAAGAATGCG ATCTGTATCA GCGTGCTGGT |
| 1361 | GTGGCTCATT GTGGTGGTGG CGATCTCCCC CATCCTCTTC |
| 1401 | TACTCAGGTA CCGGGGTCCG CAAAAACAAA ACCATCACCT |
| 1441 | GTTACGACAC CACCTCAGAC GAGTACCTGC GAAGTTATTT |
| 1481 | CATCTACAGC ATGTGCACGA CCGTGGCCAT GTTCTGTGTC |
| 1521 | CCCTTGGTGC TGATTCTGGG CTGTTACGGA TTAATTGTGA |
| 1561 | GAGCTTTGAT TTACAAAGAT CTGGACAACT CTCCTCTGAG |
| 1601 | GAGAAAATCG ATTTACCTGG TAATCATTGT ACTGACTGTT |
| 1641 | TTTGCTGTGT CTTACATCCC TTTCCATGTG ATGAAAACGA |
| 1681 | TGAACTTGAG GGCCCGGCTT GATTTTCAGA CCCCAGCAAT |
| 1721 | GTGTGCTTTC AATGACAGGG TTTATGCCAC GTATCAGGTG |
| 1761 | ACAAGAGGTC TAGCAAGTCT CAACAGTTGT GTGGACCCCA |
| 1801 | TTCTCTATTT CTTGGCGGGA GATACTTTCA GAAGGAGACT |
| 1841 | CTCCCGAGCC ACAAGGAAAG CTTCTAGAAG AAGTGAGGCA |
| 1881 | AATTTGCAAT CCAAGAGTGA AGACATGACC CTCAATATTT |
| 1921 | TACCTGAGTT CAAGCAGAAT GGAGATACAA GCCTGTGAAG |
| 1961 | GCACAAGAAT CTCCAAACAC CTCTCTGTTG TAATATGGTA |
| 2001 | GGATGCTTAA CAGAATCAAG TACTTTTCCC CTCTTTAACT |
| 2041 | TTCTAGTTTA GAAAAAAATC AAACCAAGAA AATAGTGAGT |
| 2081 | TAAAAAAATA ATAGAAGTAG AAATGCCCAC ATCCACACTT |
| 2121 | AGCTTGTTTG GGTTTGCTTT CACAGTCTCT CTTCCTTCTG |
| 2161 | ACTAGAAGTA TGTATAATAA AACAATACTA CCTAGTTAAA |
| 2201 | CATTTACTTT CTCTTTTGCC TTTAAAATGT GCAGGCTTTT |
| 2241 | CTGTTTAAAG TGTGTGTGCA CATGAGTACT GGGGCTGTTT |
| 2281 | TTGATATTAG TAATTTCTCT AAGAAAACTA GCCCCCTGCA |
| 2321 | ACTTGAGTTT GTGGTTTATC TAGCCTTTAT TGTTTTTTTA |
| 2361 | AAATCCACAG TAGGAATAAA AAATCTATAT TCTCAGAAAT |
| 2401 | ATCTAGCATG GTATATAACA AAACACTAAA CTCATCAGTT |
| 2441 | CATCCGGCAT CAGATCAATG GATCTCTGAG CGGGGTGTTT |
| 2481 | TTTTCAGTGT CTTATAAGCA TAGATGATAG TTGACTGAGT |
| 2521 | TTCTTTAGGG CATTGAATAG ACAAGTAAAG CTAATGAATT |
| 2561 | TAAAAGCCTG AAAAGTGATT GTTTTCCAGT TATTTCTGGA |
| 2601 | AAAGGTCTCA TTATATATTG GGTGCTAAAT GTTTGATGGG |
| 2641 | GAAAGCCTGC ATATATTATC GTACTGGTAA AATGCATTCA |
| 2681 | AAATAATTAA AGTGCATGTA TTTTCCTTGT AAACACCATG |
| 2721 | AGCTCTCTTA GACATCTTGT GATAAAGAGC ATTTACTTGC |
| 2761 | CCCACTGCTG TGCAATGCCT TAGGACTTTG TTTGTGTTCC |
| 2801 | AGGACAAGTG TTCACTCACA TCTGTAAAAA CAATTTTAAG |
| 2841 | AATTGCAAAT AAATTACAGA CCAAAGATTG AGTAAAGTCA |
| 2881 | AATAACTGTT AGTAAGTTGA AGGATATTGG ACAGGAGGAC |
| 2921 | AGTATTTCAG AAAAGGAGAG GTTGACAGTC ATCCACAAGG |
| 2961 | CATAGCCTCC AAGTATACTC TCAAATGTAT GAAGCAACTG |
| 3001 | GGGTGGGCAG AAGACATTTT AGAATGAGGG CTTTAGTTTA |
| 3041 | AATTAAAGTC ATGGTGGAGA AGACTCTTGC TTCCTCCAAG |
| 3081 | TGTTTGAAAA CACAAAATGC GATATGAAAA AAAAAAAAAA |
| 3121 | AA |

The corresponding protein sequence for this P2RY1 mRNA is available in the NCBI database as accession number NP 002554 (gi: 4505557). See website at ncbi.nlm.nih.gov. This sequence is provided below (SEQ ID NO:15).

| | |
|---|---|
| 1 | MTEVLWPAVP NGTDAAFLAG PGSSWGNSTV ASTAAVSSSF |
| 41 | KCALTKTGFQ FYYLPAVYIL VFIIGFLGNS VAIWMFVFHM |
| 81 | KPWSGISVYM FNLALADFLY VLTLPALIFY YFNKTDWIFG |
| 121 | DAMCKLQRFI FHVNLYGSIL FLTCISAHRY SGVVYPLKSL |
| 161 | GRLKKKNAIC ISVLVWLIVV VAISPILFYS GTGVRKNKTI |
| 201 | TCYDTTSDEY LRSYFIYSMC TTVAMFCVPL VLILGCYGLI |
| 241 | VRALIYKDLD NSPLRRKSIY LVIIVLTVFA VSYIPFHVMK |
| 281 | TMNLRARLDF QTPAMCAFND RVYATYQVTR GLASLNSCVD |
| 321 | PILYFLAGDT FRRRLSRATR KASRRSEANL QSKSEDMTLN |
| 361 | ILPEFKQNGD TSL |

Taqman gene expression systems are also available from Applied Biosystems to detect expression of Homo sapiens P2RY1 (purinergic receptor P2Y, G-protein coupled, 1(P2RY1)) mRNA. Probes and the following probes can be employed in this expression system: Pr006537214.1 (Hs01074027_s1), Pr006606184.1 (Hs00704965_s1), and combinations thereof.

Thus, in one embodiment, gene expression in skin biopsies can be detected using commercially available Taqman Gene Expression Assay reagents available from Applied Biosystems, Foster City, Calif. (see, https://www2.appliedbiosystems.com/about/; product numbers for LYVE-1 (a.k.a. XLKD1)—Hs00272659_ml, for AIF1—Hs00741549_g1, for FYB—Hs01061557_m1, for MLLT3—Hs00180312_ml, and for P2RY1—Hs01074027_s1.

With regard to length, those skilled in the art will appreciate that a probe of choice for a particular gene can be the full length coding sequence or any fragment thereof having generally at least about 8 or at least about 15 nucleotides. When the full length sequence is known, the practitioner can select any appropriate fragment of that sequence, using conventional methods. In some embodiments, multiple probes, corresponding to different portions of a given SEQ ID (molecular marker) of the invention, are used. For example, probes representing about 10 non-overlapping 20-mers can be selected from a 200-mer sequence. A skilled worker can design a suitable selection of overlapping or non-overlapping probes corresponding to each expressed polynucleotide of interest, without undue experimentation.

Accordingly, probes and primers useful for detecting expression of allograft inflammatory factor (AIF1), lymphatic hyaluronan receptor (LYVE-1), FYN binding protein (FYB), myeloid/lymphoid or mixed-lineage leukemia, translocated to, 3 (MLLT3) and/or purinergic receptor P2Y, G-protein coupled, 1(P2RY1) can readily be obtained. In addition such probes and primers are also readily obtained, for example, by identifying unique AIF1, LYVE-1, FYB, MLLT3 and/or P2RY1 sequence segments, or those complementary thereto any of SEQ ID NO:1, 3, 5, 7, 9, 11, 13 and/or 14. In many embodiment probes and primers that selectively hybridize to SEQ ID NO:1, 3, 5, 7, 9, 11, 13 and/or 14 are useful in the practice of the invention. Such AIF1, LYVE-1, FYB, MLLT3 and/or P2RY1 probes and primers can hybridize to any of nucleic acids SEQ ID NO: 1, 3, 5, 7, 9, 11, 13 and/or 14 under moderate or stringent hybridization conditions.

In some embodiments, the AIF1, LYVE-1, FYB, MLLT3 and/or P2RY1 nucleic acids, fragments, probes and/or primers are maintained in compositions. Such compositions may include any combination of, e.g., at least about 1, 2, 5, 10, 15, 20, 25, 50, 75 or 100 or more of the mentioned nucleic acids, fragments, probes and/or primers. A nucleic acid composition may comprise, consist essentially of, or consist of, AIF1, LYVE-1, FYB, MLLT3 and/or P2RY1 nucleic acids, fragments, probes and/or primers. The nucleic acid compositions may also comprise, consist essentially of, or consist of, a total of, for example, about 1, 2, 5, 10, 15, 20, 25, 50, 60, 70, 100, 150, 250, 500, 750, 1,000, 2,000, 3,000, 5,000, 7,000; 8,000; 9,000; 10,000, 11,000; 12,000; 13,000; 14,000; 15,000; 25,000, 50,000, 100,000, 200,000, 500,000, 1×10⁶, or more isolated nucleic acids.

The nucleic acid compositions of the invention may be in the form of an aqueous solution, or the nucleic acids in the composition may be immobilized on a substrate, solid surface or solid support. In some compositions of the invention, the isolated nucleic acids are in an array, such as a microarray, e.g., they are hybridizable elements on an array, such as a microarray. A nucleic acid array may further comprise, bound or double-stranded nucleic acids (e.g., those bound specifically to a probe or primer), whether on a nucleic acid array, or in an aqueous sample. In one embodiment, the nucleic acids in an array and the polynucleotides from a sample representing expressed genes have been subjected to nucleic acid hybridization under high stringency conditions (such that nucleic acids of the array that is specific for particular polynucleotides from the sample are specifically hybridized to those polynucleotides). Another embodiment is a composition comprising one or a plurality of isolated nucleic acids, each of which hybridizes specifically under high stringency conditions to part or all of a coding sequence whose expression reflects (is indicative of, is correlated with) the presence or absence of CIDP or vasculitic neuropathy.

The invention also relates to nucleic acids that are at least about 70%, 75%, 80%, 85%, 90%, 95%, 98% or 99% identical in sequence over their entire length to an AIF1, LYVE-1, FYB, MLLT3 and/or P2RY1 nucleic acid, or to a complement thereof. Conventional algorithms can be used to determine the percent identity or complementarity, e.g., as described by Lipman and Pearson (Proc. Natl. Acad Sci 80:726-730, 1983) or Martinez/Needleman-Wunsch (Nucl Acid Research 11:4629-4634, 1983).

Nucleic acids, probes and primers may be synthesized, in whole or in part, by standard synthetic methods known in the art. See, e.g., Caruthers et al. (1980) Nucleic. Acids Symp. Ser. (2) 215-233; Stein et al. (1998), Nucl. Acids Res. 16, 3209; and Sarin et al. (1988), Proc. Natl. Acad. Sci. U.S.A 85, 7448-7451. An automated synthesizer (such as those commercially available from Biosearch, Applied Biosystems) may be used. cDNA probes can be cloned and isolated by conventional methods; can be isolated from pre-existing clones, such as those from Incyte, or can be prepared by a combination of conventional synthetic methods.

### Gene Expression Assays

Changes in AIF1, LYVE-1, FYB, MLLT3 and/or P2RY1 expression levels can be detected by measuring changes in mRNA and/or a nucleic acid derived from the mRNA (e.g. reverse-transcribed cDNA, etc.). In order to measure gene expression level it is desirable to provide a nucleic acid sample for such analysis. In preferred embodiments the nucleic acid is found in or derived from a biological sample. The term “biological sample,” as used herein, refers to a sample obtained from an organism or from components (e.g., cells) of an organism. The sample may be of any biological tissue or fluid. Biological samples may also include organs or sections of tissues such as frozen sections taken for histological purposes.

It was a surprising discovery that nucleic acids derived from tissues other than neurological tissues (e.g., from skin biopsy tissues and cells) can provide effective diagnostic and/or prognostic indicators of a inflammatory neuropathies or a predilection to such a neuropathy. Thus, in some embodiments, the biological sample is a sample comprising cells of neurological origin. In other embodiments, the sample is of non-neurological origin. In certain embodiments, the biological sample comprises a skin biopsy.

The nucleic acid (e.g., mRNA, or nucleic acid derived from mRNA) is, in certain preferred embodiments, isolated from the sample according to any of a number of methods well known to those of skill in the art. Methods of isolating mRNA are well known to those of skill in the art. For example, methods of isolation and purification of nucleic acids are described in detail in by Tijssen ed., (1993) Chapter 3 of LABORATORY TECHNIQUES IN BIOCHEMISTRY AND MOLECULAR BIOLOGY: HYBRIDIZATION WITH NUCLEIC ACID PROBES, PART I. THEORY AND NUCLEIC ACID PREPARATION, Elsevier, N.Y. and Tijssen ed.

In some embodiments, the “total” nucleic acid is isolated from a given sample using, for example, an acid guanidinium-phenol-chloroform extraction method and polyA+ mRNA is isolated by oligo dT column chromatography or by using (dT)n magnetic beads (see, e.g., Sambrook et al., MOLECULAR CLONING: A LABORATORY MANUAL (2nd ed.), Vols. 1-3, Cold Spring Harbor Laboratory, (1989), or CURRENT PROTOCOLS IN MOLECULAR BIOLOGY, F. Ausubel et al., ed. Greene Publishing and Wiley-Interscience, New York (1987)).

Frequently, it is desirable to amplify the nucleic acid sample prior to assaying for expression level. Methods of amplifying nucleic acids are well known to those of skill in the art and include, but are not limited to polymerase chain reaction (PCR, see, e.g., Innis, et al., (1990) PCR PROTOCOLS. A GUIDE TO METHODS AND APPLICATION. Academic Press, Inc. San Diego,), ligase chain reaction (LCR) (see Wu and Wallace (1989) Genomics 4: 560, Landegren et al. (1988) Science 241: 1077, and Barringer et al. (1990) Gene 89: 117, transcription amplification (Kwoh et al. (1989) Proc. Natl. Acad. Sci. USA 86: 1173), self-sustained sequence replication (Guatelli et al. (1990) Proc. Nat. Acad. Sci. USA 87: 1874), dot PCR, and linker adapter PCR, etc.).

In some embodiments, where it is desirable to quantify the transcription level (and thereby expression) of factor(s) of interest in a sample, the nucleic acid sample is one in which the concentration of the nucleic acids in the sample, is proportional to the transcription level (and therefore expression level) of the gene(s) of interest. Similarly, it is preferred that the hybridization signal intensity be proportional to the amount of hybridized nucleic acid. While it is preferred that the proportionality be relatively strict (e.g., a doubling in transcription rate results in a doubling in mRNA transcript in the sample nucleic acid pool and a doubling in hybridization signal), one of skill will appreciate that the proportionality can be more relaxed and even non-linear. Thus, for example, an assay where a 5 fold difference in concentration of the target mRNA results in a 3 to 6 fold difference in hybridization intensity is sufficient for most purposes.

Where more precise quantification is required, appropriate controls can be run to correct for variations introduced in sample preparation and hybridization as described herein. In addition, serial dilutions of “standard” target nucleic acids (e.g., mRNAs) can be used to prepare calibration curves according to methods well known to those of skill in the art. Of course, where simple detection of the presence or absence of a transcript, or where large differences or changes in nucleic acid concentration are desired, no elaborate control or calibration is required.

In some embodiments, the nucleic acid sample is the total mRNA or a total cDNA isolated and/or otherwise derived from a biological sample (e.g., a sample from a skin biopsy or from a neural cell or tissue). The nucleic acid may be isolated from the sample according to any of a number of methods well known to those of skill in the art as indicated above.

Detecting and/or quantifying the transcript(s) can be routinely accomplished using nucleic acid hybridization techniques (see, e.g., Sambrook et al. supra). For example, one method for evaluating the presence, absence, or quantity of reverse-transcribed cDNA involves a “Southern Blot.” In a Southern Blot, the DNA (e.g., reverse-transcribed mRNA), typically fractionated or separated on an electrophoretic gel, is hybridized to a probe specific for the target nucleic acid. Comparison of the intensity of the hybridization signal from the target specific probe with a “control” probe (e.g. a probe for a “housekeeping” gene) provides an estimate of the relative expression level of the target nucleic acid.

Alternatively, the mRNA transcription level can be directly quantified in a Northern blot. In brief, the mRNA is isolated from a given cell sample using, for example, an acid guanidinium-phenol-chloroform extraction method. The mRNA is then electrophoresed to separate the mRNA species and the mRNA is transferred from the gel to a nitrocellulose membrane. As with the Southern blots, labeled probes can be used to identify and/or quantify the target mRNA. Appropriate controls (e.g. probes to housekeeping genes) can provide a reference for evaluating relative expression level.

An alternative means for determining the gene expression level(s) is in situ hybridization. In situ hybridization assays are well known (e.g., Angerer (1987) Meth. Enzymol 152: 649). Generally, in situ hybridization comprises the following major steps: (1) fixation of tissue or biological structure to be analyzed; (2) prehybridization treatment of the biological structure to increase accessibility of target DNA, and to reduce nonspecific binding; (3) hybridization of the mixture of nucleic acids to the nucleic acid in the biological structure or tissue; (4) post-hybridization washes to remove nucleic acid fragments not bound in the hybridization and (5) detection of the hybridized nucleic acid fragments. The reagent used in each of these steps and the conditions for use can vary depending on the particular application.

In some applications it is advisable to block the hybridization capacity of repetitive sequences. Thus, in some embodiments, tRNA, human genomic DNA, or Cot-1 DNA is used to block non-specific hybridization.

In another embodiment, amplification-based assays can be used to measure transcription level(s) of AIF1, LYVE-1, FYB, MLLT3 and/or P2RY1. In such amplification-based assays, the AIF1, LYVE-1, FYB, MLLT3 and/or P2RY1 mRNAs present in the biological sample act as template(s) in amplification reaction(s) (e.g. Polymerase Chain Reaction (PCR) or reverse-transcription PCR (RT-PCR)). In a quantitative amplification, the amount of amplification product will be proportional to the amount of template in the original sample. Comparison to appropriate controls (e.g., samples from patients that do not have an inflammatory neuropathy, provides a measure of the transcript level.

Methods of “quantitative” amplification are well known to those of skill in the art are illustrated in the Examples. For example, quantitative PCR involves simultaneously co-amplifying a known quantity of a control sequence using the same primers. This provides an internal standard that may be used to calibrate the PCR reaction. Detailed protocols for quantitative PCR are provided in Innis et al. (1990) PCR PROTOCOLS, A GUIDE TO METHODS AND APPLICATIONS, Academic Press, Inc. N.Y.). One approach, for example, involves simultaneously co-amplifying a known quantity of a control sequence using the same primers as those used to amplify the target. This provides an internal standard that may be used to calibrate the PCR reaction.

One suitable internal standard is a synthetic AW106 cRNA. The AW106 cRNA is combined with RNA isolated from the sample according to standard techniques known to those of skill in the art. The RNA is then reverse transcribed using a reverse transcriptase to provide copy DNA. The cDNA sequences are then amplified (e.g., by PCR) using labeled primers. The amplification products are separated, typically by electrophoresis, and the amount of labeled nucleic acid (proportional to the amount of amplified product) is determined. The amount of mRNA in the sample is then calculated by comparison with the signal produced by the known AW106 RNA standard. Detailed protocols for quantitative PCR are provided in Innis et al., PCR PROTOCOLS, A GUIDE TO METHODS AND APPLICATIONS (1990) Academic Press, Inc. N.Y. The known nucleic acid sequence(s) for the genes identified herein are sufficient to enable one of skill to routinely select primers to amplify any portion of the gene.

In certain embodiments, the methods of this invention can be utilized in array-based hybridization formats. Arrays typically comprise a multiplicity of different “probe” or “target” nucleic acids (or other compounds) attached to one or more surfaces (e.g., solid, membrane, or gel). In certain embodiments, the multiplicity of nucleic acids (or other moieties) is attached to a single contiguous surface or to a multiplicity of surfaces juxtaposed to each other.

Methods of making DNA arrays, including microarrays are conventional. For example, the probes may be synthesized directly on the surface; or preformed molecules, such as oligonucleotides or cDNAs, may be introduced onto (e.g., bound to, or otherwise immobilized on) the surface. Among suitable fabrication methods are photolithography, pipetting, drop-touch, piezoelectric printing (ink-j et), or the like. For some typical methods, see Ekins et al. (1999), Trends in Biotech 17, 217-218; Healey et al. (1995) Science 269, 1078-80; WO95/251116; WO95/35505; and U.S. Pat. No. 5,605,662.

Furthermore, the probes do not have to be directly bound to the substrate, but rather can be bound to the substrate through a linker group. The linker groups are typically about 6 to 50 atoms long to provide exposure to the attached nucleic acid probe. Preferred linker groups include ethylene glycol oligomers, diamines, diacids and the like. Reactive groups on the substrate surface react with one of the terminal portions of the linker to bind the linker to the substrate. The other terminal portion of the linker is then functionalized for binding the nucleic acid probe.

In an array format, a large number of different hybridization reactions can be run essentially “in parallel.” This provides rapid, essentially simultaneous, evaluation of a number of hybridizations in a single experiment. Methods of performing hybridization reactions in array based formats are well known to those of skill in the art (see, e.g., Pastinen (1997) Genome Res. 7: 606-614; Jackson (1996) Nature Biotechnology 14:1685; Chee (1995) Science 274: 610; WO 96/17958, Pinkel et al. (1998) Nature Genetics 20: 207-211).

Arrays, particularly nucleic acid arrays, can be produced according to a wide variety of methods well known to those of skill in the art. For example, in a simple embodiment, “low density” arrays can simply be produced by spotting (e.g. by hand using a pipette) different nucleic acids at different locations on a solid support (e.g. a glass surface, a membrane, etc.).

The simple spotting approach has been automated to produce high density spotted arrays (see, e.g., U.S. Pat. No. 5,807,522). This patent describes the use of an automated system that taps a microcapillary against a surface to deposit a small volume of a biological sample. The process is repeated to generate high density arrays.

Arrays can also be produced using oligonucleotide synthesis technology. Thus, for example, U.S. Pat. No. 5,143,854 and PCT Patent Publication Nos. WO 90/15070 and 92/10092 teach the use of light-directed combinatorial synthesis of high density oligonucleotide arrays. Synthesis of high density arrays is also described in U.S. Pat. Nos. 5,744,305, 5,800,992 and 5,445,934. In addition, a number of high density arrays are commercially available.

In other embodiments, nucleic acid hybridization formats such as sandwich assays and competition or displacement assays can be employed. Such assay formats are generally described in Hames and Higgins (1985) Nucleic Acid Hybridization, A Practical Approach, IRL Press; Gall and Pardue (1969) Proc. Natl. Acad. Sci. USA 63: 378-383; and John et al. (1969) Nature 223: 582-587.

Sandwich assays are commercially useful hybridization assays for detecting or isolating nucleic acid sequences. Such assays utilize a “capture” nucleic acid covalently immobilized to a solid support and a labeled “signal” nucleic acid in solution. The sample will provide the target nucleic acid. The “capture” nucleic acid and “signal” nucleic acid probe hybridize with the target nucleic acid to form a “sandwich” hybridization complex. To be most effective, the signal nucleic acid should not hybridize with the capture nucleic acid.

Typically, labeled signal nucleic acids are used to detect hybridization. Complementary nucleic acids or signal nucleic acids may be labeled by any one of several methods typically used to detect the presence of hybridized polynucleotides. One common method of detection involves the use of autoradiography with labels such as ³H, ¹²⁵I, ³⁵S, ¹⁴C, or ³²P attached to the probes or primers. Other labels include ligands that bind to labeled antibodies, fluorophores, chemi-luminescent agents, enzymes, and antibodies which can serve as specific binding pair members for a labeled ligand.

Detection of a hybridization complex may require the binding of a signal generating complex to a duplex of target and probe polynucleotides or nucleic acids. Typically, such binding occurs through ligand and anti-ligand interactions as between a ligand-conjugated probe and an anti-ligand conjugated with a signal.

The sensitivity of the hybridization assays may be enhanced through use of a nucleic acid amplification system that multiplies the target nucleic acid being detected. Examples of such systems include the polymerase chain reaction (PCR) system and the ligase chain reaction (LCR) system. Other methods recently described in the art are the nucleic acid sequence based amplification (NASBAO, Cangene, Mississauga, Ontario), Q Beta Replicase systems, or branched DNA amplifier technology commercialized by Panomics, Inc. (Fremont Calif.), and the like.

Nucleic acid hybridization simply involves providing a denatured probe and target nucleic acid under conditions where the probe and its complementary target can form stable hybrid duplexes through complementary base pairing. The nucleic acids that do not form hybrid duplexes are then washed away leaving the hybridized nucleic acids to be detected, typically through detection of an attached detectable label. It is generally recognized that nucleic acids are denatured by increasing the temperature or decreasing the salt concentration of the buffer containing the nucleic acids, or in the addition of chemical agents, or the raising of the pH. Under low stringency conditions (e.g., low temperature and/or high salt and/or high target concentration) hybrid duplexes (e.g., DNA:DNA, RNA:RNA, or RNA:DNA) will form even where the annealed sequences are not perfectly complementary. Thus specificity of hybridization is reduced at lower stringency. Conversely, at higher stringency (e.g., higher temperature or lower salt) successful hybridization requires fewer mismatches.

One of skill in the art will appreciate that hybridization conditions may be selected to provide any degree of stringency. In a preferred embodiment, hybridization is performed at low stringency to ensure hybridization and then subsequent washes are performed at higher stringency to eliminate mismatched hybrid duplexes. Successive washes may be performed at increasingly higher stringency (e.g., down to as low as 0.25×SSPE at 37° C. to 70° C.) until a desired level of hybridization specificity is obtained. Stringency can also be increased by addition of agents such as formamide. Hybridization specificity may be evaluated by comparison of hybridization to the test probes with hybridization to the various controls that can be present.

The optimal level of “stringency” of hybridization reactions is therefore readily determinable by one of ordinary skill in the art by an empirical calculation dependent upon probe length, washing temperature, and salt concentration. In general, longer probes require higher temperatures for proper annealing, while shorter probes need lower temperatures. Hybridization generally depends on the ability of denatured DNA to reanneal when complementary strands are present in an environment below their melting temperature. The higher the degree of desired homology between the probe and hybridizable sequence, the higher the relative temperature which can be used. As a result, it follows that higher relative temperatures would tend to make the reaction conditions more stringent, while lower temperatures less so. For additional details and explanation of stringency of hybridization reactions, see Ausubel et al., CURRENT PROTOCOLS IN MOLECULAR BIOLOGY, Wiley Interscience Publishers, (1995).

Moderate and stringent hybridization conditions are well known to the art, see, for example sections 0.47-9.51 of Sambrook et al. (MOLECULAR CLONING: A LABORATORY MANUAL, Cold Spring Harbor, N.Y. (1989); see also Sambrook et al., MOLECULAR CLONING: A LABORATORY MANUAL, Cold Spring Harbor, N.Y. (2001)). For example, stringent conditions are those that (1) employ low ionic strength and high temperature for washing, for example, 0.015 M NaCl/0.0015 M sodium citrate (SSC); 0.1% sodium lauryl sulfate (SDS) at 50° C., or (2) employ a denaturing agent such as formamide during hybridization, e.g., 50% formamide with 0.1% bovine serum albumin/0.1% Ficoll/0.1% polyvinylpyrrolidone/50 mM sodium phosphate buffer at pH 6.5 with 750 mM NaCl, 75 mM sodium citrate at 42° C. Another example is the use of 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% sodium dodecylsulfate (SDS), and 10% dextran sulfate at 42° C., with washes at 42° C. in 0.2×SSC and 0.1% SDS.

In some embodiments, stringent hybridization conditions are selected to be about 1° C. to 5° C. lower than the thermal melting point (Tₘ) for the specific sequence at a defined ionic strength and pH. However, moderately stringent conditions encompass hybridization and/or washing temperatures in the range of about 5° C. to about 20° C. lower than the thermal pointing point of the selected sequence.

In general, there is a tradeoff between hybridization specificity (stringency) and signal intensity. Thus, in some embodiments, the wash is performed at the highest stringency that produces consistent results, and that provides a signal intensity greater than approximately 10% of the background intensity. Thus, in a preferred embodiment, the hybridized array may be washed at successively higher stringency solutions and read between each wash. Analysis of the data sets thus produced will reveal a wash stringency above which the hybridization pattern is not appreciably altered and which provides adequate signal for the particular probes of interest. Moreover, background signal can also be reduced by the use of a blocking reagent (e.g., tRNA, sperm DNA, cot-1 DNA, etc.) during the hybridization to reduce non-specific binding. The use of blocking agents in hybridization is well known to those of skill in the art (see, e.g., Chapter 8 in P. Tijssen, supra.)

Methods of optimizing hybridization conditions are well known to those of skill in the art (see, e.g., Tijssen (1993) Laboratory Techniques in Biochemistry and Molecular Biology, Vol. 24: Hybridization With Nucleic Acid Probes, Elsevier, N.Y.).

For example, to optimize probe hybridization, the probe sequences may be examined using a computer algorithm to identify portions of genes without potential secondary structure. Such computer algorithms are well known in the art, such as OLIGO 4.06 Primer Analysis Software (National Biosciences, Plymouth, Minn.) or LASERGENE software (DNASTAR, Madison, Wis.); MACDASLS software (Hitachi Software Engineering Co, Std. South San Francisco, Calif.) and the like. These programs can search nucleotide sequences to identify stem loop structures and tandem repeats and to analyze G+C content of the sequence (those sequences with a G+C content greater than 60% are excluded). Alternatively, the probes can be optimized by trial and error. Experiments can be performed to determine whether probes and complementary target polynucleotides hybridize optimally under experimental conditions.

A “significant” increase in the expression level, as used herein, means that the value obtained in the test sample is greater than 2 standard deviations above the mean obtained with a group of control samples (p<0.05). A significant decrease in the expression level, as used herein, means that the value in the test sample is less than 2 standard deviations below the mean obtained with controls (p<0.05).

### Antibodies

According to the invention, antibodies that selectively bind to AIF1, LYVE-1, FYB, MLLT3 and/or P2RY1 can also be used to detect inflammatory neuropathies. Such antibodies can be employed in any convenient immunoassay for detecting and monitoring of inflammatory neuropathy.

The antibodies of this invention can be made by procedures known in the art. Thus, antibodies can be prepared according to conventional methods such as those described, for example, by Green et al., Production of Polyclonal Antisera, in Immunochemical Protocols (Manson, ed.), (Humana Press 1992); Coligan et al., in Current Protocols in Immunology, Sec. 2.4.1 (1992); Kohler & Milstein (1975), Nature 256, 495; Coligan et al., sections 2.5.1-2.6.7; and Harlow et al., Antibodies: A Laboratory Manual, page 726 (Cold Spring Harbor Laboratory Pub. 1988). Methods of preparing humanized or partially humanized antibodies, and antibody fragments, and methods of purifying antibodies, are conventional.

In one aspect, antibodies that selectively bind AIF1, LYVE-1, FYB, MLLT3 and/or P2RY1 may be made by using immunogens that express full length or partial sequence of AIF1, LYVE-1, FYB, MLLT3 and/or P2RY1. In another aspect, an immunogen comprising a cell that over-expresses AIF1, LYVE-1, FYB, MLLT3 and/or P2RY1 may be used.

Selected AIF1, LYVE-1, FYB, MLLT3 and/or P2RY1 epitopes or polypeptide fragments can be produced by proteolytic or other degradation of the antibodies, by recombinant methods (i.e., single or fusion polypeptides) or by chemical synthesis. Polypeptide epitopes and fragments of the antibodies, and antibody binding regions (e.g. CDRs), especially shorter polypeptides up to about 50 amino acids, are conveniently made by chemical synthesis. Methods of chemical synthesis are known in the art and are commercially available. For example, an antibody could be produced by an automated polypeptide synthesizer employing the solid phase method. See also, U.S. Pat. Nos. 5,807,715; 4,816,567; and 6,331,415. Chimeric or hybrid antibodies also may be prepared in vitro using known methods of synthetic protein chemistry, including those involving cross-linking agents.

In some embodiments, the antibodies are polyclonal. In other embodiments, the antibodies are monoclonal. Procedures for making polyclonal and monoclonal antibodies are available in the art.

The route and schedule of immunization of the host animal are generally in keeping with established and conventional techniques for antibody stimulation and production. It is contemplated that any mammalian subject including humans or antibody producing cells therefrom can be manipulated to serve as the basis for production of mammalian, including human, hybridoma cell lines. Typically, the host animal is inoculated intraperitoneally, intramuscularly, orally, subcutaneously, intraplantar, and/or intradermally with an amount of immunogen.

Hybridomas can be prepared from the lymphocytes and immortalized myeloma cells using the general somatic cell hybridization technique of Kohler, B. and Milstein, C. (1975) Nature 256:495-497 or as modified by Buck, D. W., et al., In Vitro, 18:377-381 (1982). Available myeloma lines, including but not limited to X63-Ag8.653 and those from the Salk Institute, Cell Distribution Center, San Diego, Calif., USA, may be used in the hybridization. Generally, the technique involves fusing myeloma cells and lymphoid cells using a fusogen such as polyethylene glycol, or by electrical means well known to those skilled in the art. After the fusion, the cells are separated from the fusion medium and grown in a selective growth medium, such as hypoxanthine-aminopterin-thymidine (HAT) medium, to eliminate unhybridized parent cells. Any of the media described herein, supplemented with or without serum, can be used for culturing hybridomas that secrete monoclonal antibodies. As another alternative to the cell fusion technique, EBV immortalized B cells may be used to produce the monoclonal antibodies of the subject invention. The hybridomas are expanded and subcloned, if desired, and supernatants are assayed for anti-immunogen activity by conventional immunoassay procedures (e.g., radioimmunoassay, enzyme immunoassay, or fluorescence immunoassay).

Hybridomas that may be used as source of antibodies encompass all derivatives, progeny cells of the parent hybridomas that produce monoclonal antibodies specific for AIF1, LYVE-1, FYB, MLLT3 and/or P2RY1, or a portion thereof.

Hybridomas that produce such antibodies may be grown in vitro or in vivo using known procedures. The monoclonal antibodies may be isolated from the culture media or body fluids, by conventional immunoglobulin purification procedures such as ammonium sulfate precipitation, gel electrophoresis, dialysis, chromatography, and ultrafiltration, if desired. Undesired activity if present, can be removed, for example, by running the preparation over adsorbents made of the immunogen attached to a solid phase and eluting or releasing the desired antibodies off the immunogen.

A host animal can be immunized with a AIF1, LYVE-1, FYB, MLLT3 and/or P2RY1 polypeptide, or a fragment containing a selected epitope or target amino acid sequence. The polypeptide, epitope or target amino acid sequence can be conjugated to a protein that is immunogenic in the species to be immunized, e.g., keyhole limpet hemocyanin, serum albumin, bovine thyroglobulin, or soybean trypsin inhibitor using a bifunctional or derivatizing agent, for example maleimidobenzoyl sulfosuccinimide ester (conjugation through cysteine residues), N-hydroxysuccinimide (through lysine residues), glutaradehyde, succinic anhydride, SOCl₂, or R₁N═C═NR, where R and R₁ are different alkyl groups, can yield a population of antibodies.

In another alternative, the antibodies can be made recombinantly using procedures that are well known in the art. In one embodiment, a polynucleotide comprising a sequence encoding the variable and light chain regions of a previously identified antibody is cloned into a vector for expression or propagation in a host cell (e.g., CHO cells). The sequence encoding the antibody of interest may be maintained in a vector in a host cell and the host cell can then be expanded and frozen for future use. Methods for expressing antibodies recombinantly in plants or milk have been disclosed. See, for example, Peeters et al. (2001) Vaccine 19:2756; Lonberg, N. and D. Huszar (1995) Int. Rev. Immunol 13:65; and Pollock et al. (1999) J Immunol Methods 231:147. Methods for making derivatives of antibodies, e.g., humanized, single chain, etc. are known in the art.

For example, DNA encoding the monoclonal antibodies is readily isolated and sequenced using conventional procedures (e.g., by using oligonucleotide probes that are capable of binding specifically to genes encoding the heavy and light chains of the monoclonal antibodies). The hybridoma cells serve as a preferred source of such DNA. Once isolated, the DNA may be placed into expression vectors (such as expression vectors disclosed in PCT Publication No. WO 87/04462), which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. See, e.g., PCT Publication No. WO 87/04462. The DNA also may be modified, for example, by substituting the coding sequence for human heavy and light chain constant domains in place of the homologous murine sequences, Morrison et al., Proc. Nat. Acad. Sci. 81:6851 (1984), or by covalently joining to the immunoglobulin coding sequence all or part of the coding sequence for a non-immunoglobulin polypeptide. In that manner, “chimeric” or “hybrid” antibodies are prepared that have the binding specificity for AIF1, LYVE-1, FYB, MLLT3 and/or P2RY1 polypeptides.

The invention also encompasses single chain variable region fragments (“scFv”) of antibodies that can selectively bind to AIF1, LYVE-1, FYB, MLLT3 and/or P2RY1 polypeptides. Single chain variable region fragments are made by linking light and/or heavy chain variable regions by using a short linking peptide. See, e.g., Bird et al. (1988) Science 242:423-426. An example of a linking peptide is (GGGGS)₃ (SEQ ID NO:16), which bridges approximately 3.5 nm between the carboxy terminus of one variable region and the amino terminus of the other variable region. Linkers of other sequences have been designed and used (Bird et al. (1988)). Linkers can in turn be modified for additional functions, such as attachment of the antibody to solid supports. The single chain variants can be produced either recombinantly or synthetically. For synthetic production of scFv, an automated synthesizer can be used. For recombinant production of scFv, a suitable plasmid containing polynucleotide that encodes the scFv can be introduced into a suitable host cell, either eukaryotic, such as yeast, plant, insect or mammalian cells, or prokaryotic, such as E. coli. Polynucleotides encoding the scFv of interest can be made by routine manipulations such as ligation of polynucleotides. The resultant scFv can be isolated using standard protein purification techniques known in the art.

Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger, P., et al. (1993) Proc. Natl. Acad. Sci. USA 90:6444-6448; Poljak, R. J., et al. (1994) Structure 2:1121-1123).

The binding affinity of an antibody to an AIF1, LYVE-1, FYB, MLLT3 and/or P2RY1 polypeptide can be about 0.10 to about 10 nM, about 0.15 to about 7.5 nM and about 0.2 to about 5.0 nM. In some embodiments, the binding affinity is about 2 pM, about 5 pM, about 10 pM, about 15 pM, about 20 pM, about 40 pM, or greater than about 40 pM. In one embodiment, the binding affinity is between about 2 pM and 22 pM. In other embodiments, the binding affinity is less than about 10 nM, about 5 nM, about 4 nM, about 3.5 nM, about 3 nM, about 2.5 nM, about 2 nM, about 1.5 nM, about 1 nM, about 900 pM, about 800 pM, about 700 pM, about 600 pM, about 500 pM, about 400 pM, about 300 pM, about 200 pM, about 150 pM, about 100 pM, about 90 pM, about 80 pM, about 70 pM, about 60 pM, about 50 pM, about 40 pM, about 30 pM, about 10 pM. In some embodiments, the binding affinity is about 10 nM. In other embodiments, the binding affinity is less than about 10 nM. In other embodiments, the binding affinity is about 0.1 nM or about 0.05 nM. In other embodiments, the binding affinity is less than about 0.1 nM or less than about 0.07 nM. In other embodiments, the binding affinity is any of about 10 nM, about 5 nM, about 4 nM, about 3.5 mM, about 3 nM, about 2.5 mM, about 2 nM, about 1.5 nM, about 1 nM, about 900 pM, about 800 pM, bout 700 pM, about 600 pM, about 500 pM, about 400 pM, about 300 pM, about 200 pM, about 150 pM, about 100 pM, about 90 pM, about 80 pM, about 70 pM, about 60 pM, about 50 pM, about 40 pM, about 30 pM, about 10 pM to any of about 2 pM, about 5 pM, about 10 pM, about 15 pM, about 20 pM, or about 40 pM. In some embodiments, the binding affinity is any of about 10 nM, about 5 nM, about 4 nM, about 3.5 nM, about 3 nM, about 2.5 nM, about 2 nM, about 1.5 nM, about 1 nM, about 900 pM, about 800 pM, bout 700 pM, about 600 pM, about 500 pM, about 400 pM, about 300 pM, about 200 pM, about 150 pM, about 100 pM, about 90 pM, about 80 pM, about 70 pM, about 60 pM, about 50 pM, about 40 pM, about 30 pM, about 10 pM. In still other embodiments, the binding affinity is about 2 pM, about 5 pM, about 10 pM, about 15 pM, about 20 pM, about 40 pM, or greater than about 40 pM.

The binding affinity of the antibody to AIF1, LYVE-1, FYB, MLLT3 and/or P2RY1 polypeptide can be determined using methods available in the art. One way of determining binding affinity of antibodies to AIF1, LYVE-1, FYB, MLLT3 and/or P2RY1 polypeptide is by measuring affinity of monofunctional Fab fragments of the antibody. To obtain monofunctional Fab fragments, an antibody (for example, IgG) can be cleaved with papain or expressed recombinantly. The affinity of the Fab fragment of an antibody can be determined by surface plasmon resonance (BIAcore3000™ surface plasmon resonance (SPR) system, BIAcore, INC, Piscataway N.J.). This protocol is suitable for use in determining binding affinity of an antibody to AIF1, LYVE-1, FYB, MLLT3 and/or P2RY1.

The antibodies can be bound to many different solid surfaces. Examples of well-known carriers include polypropylene, polystyrene, polyethylene, dextran, nylon, amylases, glass, natural and modified celluloses, polyacrylamides, agaroses and magnetite. Those skilled in the art will know of other suitable solid supports for binding or displaying antibodies, or will be able to ascertain such, using routine experimentation.

### Immunoassays

Any available immunoassay can be employed to detect AIF1, LYVE-1, FYB, MLLT3 and/or P2RY1 polypeptides in samples. For example, one or more the antibodies that selectively bind AIF1, LYVE-1, FYB, MLLT3 and/or P2RY1 polypeptides can be attached to a solid surface. The presence or level of AIF1, LYVE-1, FYB, MLLT3 and/or P2RY1 is determined using an immunoassay or an immunohistochemical assay.

A non-limiting example of an immunoassay suitable for use in the method of the present invention includes an enzyme-linked immunosorbent assay (ELISA). Examples of immunohistochemical assays suitable for use in the method of the present invention include, but are not limited to, immunofluorescence assays such as direct fluorescent antibody assays, indirect fluorescent antibody (IFA) assays, anticomplement immunofluorescence assays, and avidin-biotin immunofluorescence assays. Other types of immunohistochemical assays include immunoperoxidase assays. Antibodies that selectively bind to AIF1, LYVE-1, FYB, MLLT3 and/or P2RY1 can also be used in assay methods, such competitive binding assays, direct and indirect sandwich assays, and immunoprecipitation assays. Zola, Monoclonal Antibodies: A Manual of Techniques, pp. 147-158 (CRC Press, Inc. 1987). The antibodies can also be used in assays for detecting AIF1, LYVE-1, FYB, MLLT3 and/or P2RY1 that involve use of surface plasmon resonance (BIAcore3000™ surface plasmon resonance (SPR) system, BIAcore, INC, Piscataway N.J.).

### Types of Inflammatory Neuropathies

Many types of inflammatory neuropathies can be detected using the methods of the invention. Examples of inflammatory neuropathies that can be detected using the methods of the invention include infectious neuropathies (with a specific casual agent) and autoimmune neuropathies. Examples of infectious inflammatory neuropathies include Lyme disease, HIV/AIDS, Leprosy, Herpes Zoster (Shingles), Hepatitis B, Hepatitis C. Examples of autoimmune or possibly infectious (but with no specific causal infectious agent identified) inflammatory neuropathies include Sarcoidosis, Guillain-Barré Syndrome/Acute Inflammatory Demyelinating Polyneuropathy (AIDP), Chronic Inflammatory Demyelinating Polyneuropathy (CIDP), Vasculitis (e.g., Polyarteritis Nodosa (PAN), Rheumatoid Arthritis, Systemic Lupus Erythematosus (Lupus) Sjögren's Syndrome), Celiac Disease, Multifocal Motor Neuropathy (MNN), Peripheral Neuropathy Associated with Protein Abnormalities (e.g., Monoclonal Gammopathy, Amyloidosis, Cryoglobulinemia and/or POEMS). The present methods can be used to detect any such inflammatory neuropathy.

In some embodiments, the method is used to detect Chronic Inflammatory Demyelinating Polyneuropathy (CIDP).

### Detection Methods

Any available method for detecting inflammatory neuropathies using the AIF1, LYVE-1, FYB, MLLT3 and/or P2RY1 probes and primers

inflammatory neuropathies s

### Kits and Microarrays

Also contemplated by the present invention are various diagnostic and test kits. Such kits may be used for determining whether a patient has inflammatory neuropathy or is at risk of developing inflammatory neuropathy. In some embodiments, the kit is used for monitoring the progression or status of an existing inflammatory neuropathy condition. The kit comprises a reagent or assay device for assessing expression of the marker genes of interest (AIF1, LYVE-1, FYB, MLLT3 and/or P2RY1). The reagent or assay device for assessing expression of AIF1, LYVE-1, FYB, MLLT3 and/or P2RY1 can be one or more nucleic acid probes that with hybridize to an AIF1, LYVE-1, FYB, MLLT3 and/or P2RY1 mRNA (e.g., to one of the AIF1, LYVE-1, FYB, MLLT3 and/or P2RY1 nucleic acids described herein). The nucleic acid probe(s) binds specifically with at least one AIF1, LYVE-1, FYB, MLLT3 and/or P2RY1 nucleic acid (e.g., mRNA) or a fragment of the nucleic acid. The kit may further comprise a plurality of probes, wherein each of the probes binds specifically with a AIF1, LYVE-1, FYB, MLLT3 and/or P2RY1 nucleic acid, or a fragment of the nucleic acid.

Alternatively, the reagent or assay device for assessing expression of AIF1, LYVE-1, FYB, MLLT3 and/or P2RY1 can be one or more (or a plurality) of antibodies, antibody derivatives, or antibody fragments wherein the antibodies bind specifically with AIF1, LYVE-1, FYB, MLLT3 and/or P2RY1 marker protein, or a fragment of any of these proteins.

The invention is also directed to a kit for assessing or monitoring the presence of inflammatory neuropathy, wherein the kit comprises at least one microarray. The microarray is used for measuring gene expression of AIF1, LYVE-1, FYB, MLLT3 and/or P2RY1 genes that are differentially expressed in patients with inflammatory neuropathies.

The microarray can comprise at least two nucleic acid probes selected from AIF1, LYVE-1, FYB, MLLT3 and/or P2RY1 probes or at least two antibodies selected from AIF1, LYVE-1, FYB, MLLT3 and/or P2RY1 antibodies.

In some embodiments, the microarray of the invention consists of AIF1, LYVE-1, FYB, MLLT3 and/or P2RY1 polynucleotide probes, optionally with control probes. The control probes can be selected from various housekeeping genes. Examples of housekeeping genes that can be used as control probes include GAPDH, B-Actin, 18S, HMBS, HPRT, PGK1, STST1, TBP, UBC polynucleotide probes, and combinations thereof.

In other embodiments, the microarray comprises at least 5, 10, 15, 25, or 50 polynucleotide probes, wherein, in each such embodiment, each of the expressly enumerated number of probes has a distinct sequence from the at least two probes selected from AIF1, LYVE-1, FYB, MLLT3 and/or P2RY1 probes. In some embodiments, the microarray is prepared using a plurality probes that hybridize to different sections of each of the genes selected from the group consisting of AIF1, LYVE-1, FYB, MLLT3 and/or P2RY1 probes. For example, the microarray may comprise 5, 10, 15, 20, 25, 30, 40, 45, 50 or more probes that hybridize to different parts of one or more of the AIF1, LYVE-1, FYB, MLLT3 and/or P2RY1 mRNA sequences. Thus, the microarray may comprise an equal or different number of distinct probes that hybridize to different parts of the AIF1 mRNA, and may comprise an equal or different number of distinct probes that hybridize to different parts of the LYVE-1 mRNA, and may also comprise an equal or different number of distinct probes that hybridize to different parts of the FYB mRNA, and may further comprise an equal or different number of distinct probes that hybridize to different parts of the MLLT3 mRNA, and may further comprise an equal or different number of distinct probes that hybridize to different parts of the P2RY1 mRNA. The microarray may therefore comprise probes directed to each of the mRNAs selected from the group consisting of AIF1, LYVE-1, FYB, MLLT3 and/or P2RY1. Alternatively, the microarray may comprise probes directed to only some of the mRNAs from this group. In specific embodiments, the primers or probes may be between 5 to 25 bases in length. Of course longer probes also may be used.

### General Procedures

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning. A Laboratory Manual, second edition (Sambrook et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-1998) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel et al., eds., 1987); PCR. The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J.B. Lippincott Company, 1993), where the contents of each of these publications are hereby specifically incorporated herein in their entireties.

The following examples further illustrate the invention.

### Example 1

### Expression of AIF1 and CLCA2 in Skin Biopsies

### Methods

To date, five (5) CIDP patients and seven (7) normal controls have been examined. Five patients with Charcot Marie Tooth (CMT) syndrome, a non-inflammatory peripheral neuropathy, were included as additional controls. All volunteers were recruited at The Neuropathy Center of the Cornell Weill Medical College.

RNA extracted from 2 mm punch skin biopsies from the forearm was subjected to RT-PCR in triplicate wells to determine expression of AIF1, CLCA2, MSR1 (Macrophage scavenger receptor-1), NQO1 (Quinone I), NRID1 (Nuclear receptor subfamily I), SCD (Steroyl-CoA desaturase), TAC1 (Tachykinin-1), S100B (Schwann cell protein), P0 (positive control for presence of myelin), and GAPDH (endogenous control) using specific primer-probe sets and Taqman reagents from Applied Biosystems, CA.

Data analysis for relative quantification of gene expression was performed with the ABI PRISM 7700 SDS program. The threshold cycle number (Ct) for GAPDH was subtracted from the Ct for each target gene to determine each Delta Ct value. Each patient Delta Ct value was normalized against normal controls by subtracting the average Delta Ct value of the normal group from each patient Delta Ct to obtain the Delta Delta Ct (DD Ct) values. The fold difference (FD) of gene expression over normal controls for each patient sample was calculated as FD=^{2-DD Ct}, where FD<1 indicates decreased expression; FD=1 indicates no difference; and FD>1 indicates increased expression (Applied Biosystems, 2004).

### Results

To test for the specificity of molecular markers associated with inflammatory neuropathy, gene expression in CIDP patients was compared to that in CMT patients. Significant increases of 2.1 to 7.9 fold over normal values were found in 4 of 5 CIDP patients for the inflammatory marker AIF1 (see Table 1). Increased expression of CLCA2 was also observed in 3 of 5 CIDP biopsies. It remains to be determined if the patient (R8) with low AIF1 and CLCA2 was in remission at the time of the biopsy. Values for these two proteins were similar to normal controls among the CMT patients. Elevations were also detected for the other markers tested but variation was relatively broad among the small numbers of CIDP samples and overlapped with values for CMT.

TABLE 1

| | | Mean Fold Difference over Normal Controls | |
|---|---|---|---|
| Diagnosis | Patient | AIFl | CLCA2 |
| CMT (5)^{a} | | 0.98 (0.06-1.89) | 0.95 (0.10-1.81) |
| CIDP | R9 | 7.85 (6.70-9.20) | 2.74 (2.36-3.19) |
| CIDP | R10 | 2.40 (2.03-2.85) | 7.04 (6.28-7.90) |
| CIDP | R22 | 2.07 (1.93-2.22) | 0.86 (0.79-0.94) |
| CIDP | R4 | 4.30 (3.89-4.77) | 2.14 (1.93-2.38) |
| CIDP | R8 | 0.40 (0.29-0.56) | 0.52 (0.47-0.58) |

^{a}The values for 5 CMT patients were averaged to provide a baseline value for comparison to CIDP individuals.
^{b}The upper and lower limits represent the mean ± 2 standard deviations (95% confidence limits, p < 0.05)

As shown by the data in Table 1, skin biopsies from the forearm are a suitable alternative to the potentially more risky and damaging procedure of taking sural nerve biopsies for assessment of gene expression to aid in the diagnosis of CIDP or other inflammatory neuropathies. The RT-PCR results shown in Table 1 confirm that at least one of the molecular markers, AIF1, previously found to be elevated in sural nerve biopsies through the more costly microarray analysis is a promising diagnostic indicator for CIDP and related neuropathies.

### Example 2

### Increased Gene Expression of AIF1, LYVE-1, and FYB in Skin Biopsies from Chronic Inflammatory Demyelinating Polyneuropathy

Gene expression analysis previously identified molecular markers that are up-regulated in sural nerve biopsies from patients with chronic inflammatory demyelinating polyneuropathy (CIDP). This Example illustrates that the expression of three markers, allograft inflammatory factor 1 (AIF1), lymphatic hyaluronan receptor (LYVE-1), and FYN binding protein (FYB), all involved in inflammatory processes, were also elevated in punch skin biopsies from patients with CIDP as compared to Charcot-Marie-Tooth disease, diabetic neuropathy, or normal subjects. Therefore, the relative expression of these three markers in minimally invasive skin biopsies can help distinguish patients with CIDP from those with non-inflammatory neuropathies.

### Introduction

Chronic inflammatory demyelinating polyneuropathy (CIDP) is an autoimmune disease that targets the myelin sheaths of the peripheral nerves (Koller et al, 2005). Studies of differential gene expression in sural nerve biopsy studies from patients with CIDP reveal increased expression of mRNAs that encode for inflammatory mediators (Renaud et al, 2005). In this study, quantitative PCR was used to compare the expression of several mRNAs from the skin of patients with CIDP, to that from normal subjects and patients with hereditary or diabetic neuropathy.

### Patients and Methods

Patient and normal volunteers were recruited at The Neuropathy Center of the Cornell Weill Medical College, with IRB approval and the patients' informed consent. Six patients had CIDP, eight had hereditary demyelinating neuropathy or Charcot-Marie-Tooth disease type I (CMT1), five had diabetic neuropathy (DN), and seven were healthy subjects. CIDP was diagnosed according to guidelines issued by the Joint Task Force of the EFNS and the PNS (2005). Patients with diabetic neuropathy had type II diabetes as defined by the American Diabetes Association, and a distal axonal neuropathy. The diagnosis of CMT type I (Shy et al, 2002), was made on the basis of genetic testing (Athena Diagnostics Inc, Wooster Mass.).

Punch skin biopsies, approximately 2 mm³, were obtained from the forearm and immediately immersed in RNALater solution (Ambion, Inc., Austin, Tex.) for stabilization. DNAse-treated RNA was quantitatively reversed transcribed and amplified according to the protocol of the WT-Ovation RNA Amplification kit (NuGen Technologies, Inc., San Carlos, Calif.). Equal amounts of total RNA from normal subjects were pooled as a single control unit.

Real-time PCR (RT-PCR) was performed in triplicate wells with standard thermocycling settings in the ABI 7900HT instrument (Applied Biosystems, Foster City, Calif.) with 12 ng cDNA, 4 ul TaqMan 2× Master Mix (Applied Biosystems), and 0.4 ul TaqMan primer-probe per 8 ul reaction volume. TaqMan gene expression assay primer-probe sets for the following genes were used: AIF1 (allograft inflammatory factor-1; ABI product no. Hs00741549_g1), LYVE-1 (XLKD1; hyaluronan receptor; ABI product no. Hs00272659_m1), FYB (ADAP; FYN binding protein, ABI product no. Hs01061557_m1), P0 (myelin protein 0), and GAPDH (endogenous control). RT-PCR data were normalized to GAPDH and relative mRNA expression was calculated with the ΔΔCt method using the pooled normal samples as the calibrator (Livak and Schmittgen, 2001). Also assayed were expressions of CLCA2, MSR1, NQO1, NRID1, SCD, TAC1, HLA-DQB1, IL1RB, MARCO, and PRG2 (BMPG), as increased expression of these genes was reported in CIDP or vasculitis sural nerve biopsies (Renaud et al, 2005).

Statistical analysis was performed with the GraphPad Instat software (Instant Statistics, GraphPad Software, San Diego, Calif.) using the nonparametric Krustal-Wallis test for unpaired data.

### Results

Expression of three genes assayed, AIF1, LYVE-1, and FYB were most consistently elevated in CIDP relative to the CMT1 or DN groups (Table 2).

TABLE 2
Fold Difference in Expression Relative to Normal Controls

| Diagnosis | Patient | AIF1 | LYVE-1 | FYB |
|---|---|---|---|---|
| CIDP | R4 | 4.30 | 2.92 | 2.17 |
| (n = 6) | R9 | 7.85 | 2.84 | 7.09 |
| | R10 | 2.40 | 2.76 | 1.92 |
| | R22 | 2.07 | 2.44 | 1.76 |
| | R24 | 2.38 | 3.56 | 0.89 |
| | R31 | 3.16 | 1.79 | 1.51 |
| Mean ± std dev: | | 3.69 ± 2.19 | 2.72 ± 0.58 | 2.56 ± 2.26 |
| CMT1 | R11 | 0.82 | 1.02 | 0.39 |
| (n = 8) | R12 | 1.15 | 0.60 | 0.92 |
| | R13 | 0.94 | 0.73 | 0.46 |
| | R18 | 0.37 | 0.69 | 0.05 |
| | R19 | 1.62 | 0.90 | 1.15 |
| | R23 | 0.34 | 1.00 | 0.59 |
| | R25 | 0.61 | 0.91 | 2.96 |
| | R33 | 1.35 | 1.01 | 0.67 |
| Mean ± std dev: | | 0.90 ± 0.46 | 0.86 ± 0.16 | 0.90 ± 0.90 |
| DN | R14 | 1.07 | 0.96 | 0.68 |
| (n = 5) | R16 | 2.06 | 0.05 | 3.95 |
| | R17 | 1.49 | 0.84 | 1.58 |
| | R20 | 0.31 | 0.60 | 0.36 |
| | R35 | 1.35 | 1.22 | 0.36 |

| | | | |
|---|---|---|---|
| Mean ± std dev: | 1.26 ± 0.64 | 0.73± 0.44 | 1.39 ± 1.52 |
| p value: | | | |
| CIDP vs. CMT1 | 0.0007 * | 0.0007 * | 0.0293 * |
| CIDP vs. DN | 0.0043 * | 0.0043 * | 0.1775 |
| CMT1 vs. DN | 0.3795 | 0.6603 | 0.6329 |

* statistically significant (unpaired, 2 tailed, non-parametric Mann-Whitney test)

As shown in Table 2, all three markers were increased greater than 1.5 fold over normal controls in five of the six CIDP samples, and AIF1 and LYVE-1 were increased in all six. One of the CMT1 samples showed an increase in FYB but none had increased expression of both AIF1 and LYVE-1. Of the five samples from diabetic neuropathy, one showed an increase in AIF1 and FYB, but not in LYVE-1.

No significant differences in the expressions of CLCA2, MSR1, NQO1, NRID1, SCD, TAC1, HLA-DQB1, IL1RB, MARCO, or PRG2 (BMPG), were observed between the different patient groups and normal controls (data not shown). P0 was detected in all samples, indicating that myelinated nerve tissue was present in the skin biopsies, although expression was generally lower among all three patient groups compared to normal controls, probably reflecting a decrease of myelinated fibers in the neuropathy patients.

### Discussion

As demonstrated by the data shown here, the expression of AIF1, LYVE-1 and FYB was increased in skin biopsies of patients with CIDP. While the expression of AIF1 and FYB was previously reported to be up-regulated in sural nerve biopsies from patients with both CIDP and vasculitis (Renaud et al, 2005), no one has previously shown that CIDP and other inflammatory neuropathies can be diagnosed using skin biopsies.

AIF1 is produced by activated macrophages in transplant rejection and autoimmune disorders (Liu et al, 2007; Orsmark et al, 2007), and LYVE-1 is a marker for lymphatic endothelium which is also expressed by activated bone marrow and tissue macrophages (Schledzewski et al, 2006). FYB, also called ADAP (Adhesion and Degranulation Promoting Adoptor Protein), mediates signaling from T-cell antigen receptors to integrins, leading to enhanced cellular adhesion (Peterson 2003). Their increased expression in skin biopsies of patients with CIDP could reflect the presence of terminal nerve fibers, including from myelinated nerves, in skin (Provitera et al, 2007), or result from a systemic inflammatory reaction.

Expression of AIF1, LYVE-1, and FYB, appear to be unrelated to demyelination, as it is not increased in skin biopsies from patients with hereditary demyelinating neuropathies caused by genetic defects. AIF1 and FYB were also elevated in one of five diabetic neuropathy skin biopsies, where milder inflammatory changes have been described (Younger at al, 1996; Rosoklija et al, 2000). Of the CMT1 samples, one had mild elevation of AIF1 and another of FYB, but none had elevation of two or more markers.

The diagnosis of CIDP is based on the clinical presentation and results of electrodiagnostic studies. A nerve biopsy, however, may be required to confirm the diagnosis in atypical cases, and CIDP can sometimes occur in patients with otherwise typical diabetic or hereditary neuropathy (Haq et al, 2003; Ginsberg et al, 2004). Although elevations of AIF1 and LYVE-1 in skin are not specific for CIDP or inflammatory neuropathy, and are likely to be increased in inflammatory conditions that involve the skin, determination of mRNA levels in skin biopsies may help distinguish patients with possible CIDP, from those with non-inflammatory neuropathy such as CMT1.

### Example 3

### Gene Expression of AIF1 and XLKD1/LYVE-1 in Chronic Inflammatory Demyelinating Polyneuropathy (CIDP) Skin Biopsies

Gene expression analysis previously identified molecular markers that are up-regulated in CIDP sural nerves. Using quantitative real-time PCR (RT-PCR), we analyzed the expression of some of the same markers in forearm skin. Samples from patients with CIDP, obtained by punch biopsy, were compared to those from Charcot Marie Tooth (CMT) disease, diabetic neuropathy, or normal controls. Two genes, AIF1 and XLKD1/LYVE-1, both involved in inflammatory processes, were most consistently elevated in CIDP relative to the other groups. Expression of both was significantly increased in the CIDP group as compared to CMT (p<0.05). AIF1 was increased over normal controls in six of six CIDP patients (2.1-7.9 fold), compared to one of eight CMT patients. Similarly, XLDK1/LYVE-1 was elevated in all six CIDP patients (1.8-3.6 fold) but in none of the CMT patients or of the five diabetic neuropathy samples examined.

These results indicate that determination of expression of AIF1 and XLKD1/LYVE-1 in punch skin biopsies can help distinguish patients with CIDP from those with non-inflammatory neuropathies.

### Example 4

### P2RY1 and MLLT3 are also CIDP Markers

As described above in Examples 1-3, quantitative real-time PCR (qPCR) showed that 3 markers, allograft inflammatory factor 1 (AIF1), lymphatic hyaluronan receptor (LYVE-1/XLKD1), and FYN binding protein (FYB), all involved in inflammatory processes, were also elevated in punch skin biopsies from patients with CIDP as compared to Charcot-Marie-Tooth Type 1 (CMT1) disease or normal subjects. In this Example, gene microarray profiling of skin biopsies was employed to reveal that an additional two genes are differentially over-expressed in CIDP: P2RY1 (purinergic receptor P2Y, G-protein coupled, 1) which may be active in inflammation and immunity, and MLLT3 (myeloid/lymphoid or mixed lineage leukemia translocated to, 3). Also as shown in the Example, the cumulative fold change in expression over normal expression, as determined by qPCR of all 5 genes, is significantly elevated in CIDP relative to CMT1 and may serve as a useful index to may help distinguish patients with CIDP from those with non-inflammatory neuropathies.

### Materials and Methods

Patients: Patient and normal volunteers were recruited at The Neuropathy Center of the Cornell Weill Medical College, with IRB approval and the patient informed consent. Eleven patients with CIDP, 8 with hereditary demyelinating neuropathy or Charcot-Marie-Tooth disease type I (CMT1), and 7 healthy subjects were included in this study. CIDP was diagnosed according to the EFNS and PNS Joint Task Force guidelines (2005). The diagnosis of CMT type I, was made on the basis of genetic testing (Athena Diagnostics Inc, Wooster Mass.) and clinical observation (Shy et al, 2002).

Skin biopsies and RNA preparation: Punch skin biopsies, approximately 2 mm³, were obtained from the forearm and immediately immersed in RNALater solution (Applied Biosystems, Foster City) for RNA preservation. The samples were homogenized mechanically in 1 ml Qiagen RLT lysis solution, extracted at 4 C with 0.5 ml phenol:chloroform, pH 4.7, then purified and DNAse treated on a spin column using the RNeasy Mini-kit (Qiagen, Valencia, Calif.). RNA was quantified on a Nanodrop ND-1000 spectrophotometer (Thermo Scientific, Wilmington, Del.) and assayed for quality on a 2100 Bioanalyzer (Agilent Technologies, Foster City, Calif.).

Microarray gene profiling: RNA from 10 CIDP, 6 CMT1 and 5 normal samples were quantitatively reverse transcribed and amplified, then labeled and fragmented with the Nugen Ovation RNA Amplification and the FL-Ovation cDNA Biotin Module V2 kits, respectively (NuGen Technologies, Inc., San Carlos, Calif.). The samples were hybridized to Affymetrix Human U133 Plus 2.0 microarray chips (Affymetrix, Santa Clara, Calif.) and signal intensities were read in the Hewlett-Packard G2500A Gene Array Scanner.

Microarray data analysis: Chip data was imported into the GeneSpring GX 7.3 program (Agilent Technologies, Foster City, Calif.). Signal values less than 0.01 were set to 0.01, arrays were normalized to the 50th percentile, and individual genes were normalized to the median. Normalized data was then filtered to retain genes flagged as present or marginal in all of the CIDP samples. One-way ANOVA (variances not assumed to be equal) with p<0.05, comparing each neuropathy group with the normal group or with each other, followed by filtration for greater or less than 1.5 fold differences, was applied to determine potential differential expression.

Classification by Gene Ontology (GO) Consortium assigned biologic processes, pathway and network analyses through the GeneSpring GS and Ingenuity Pathway Analysis (Ingenuity Systems, Redwood City Calif.) software further identified genes of potential interest.

Quantitative real-time PCR (qPCR): DNAse-treated RNA was quantitatively reversed-transcribed and amplified according to the protocol of the WT-Ovation RNA Amplification kit (NuGen Technologies, Inc., San Carlos, Calif.). Equal amounts of total RNA from 7 normal subjects were pooled as a single control unit. cDNA was purified though Zymo DCC-25 spin columns (Zymo Research, Orange, Calif.).

Quantitative PCR was performed in triplicate wells of 384-well plates in the ABI 7900HT instrument (Applied Biosystems, Foster City, Calif.) with 12 ng cDNA, 5 ul TaqMan 2× Gene Expression Master Mix (Applied Biosystems), and 0.5 ul gene-specific 20× TaqMan Gene Expression Assay primer-probe per 10 μl reaction volume. Thermocycling settings were: 50° C., 2 min; 95° C., 10 min; 40 cycles of 95° C., 2 sec, 60° C., 1 min.

To identify optional endogenous gene controls, a panel of 10 Taqman primer-probe sets was tested either individually or in various combinations for normalization of target gene data. Genes with the most stable expression among all tissue samples were determined through the geNorm VBA appletsoftware (http://medgen.ugent.be/˜jvdesomp/genorm/) (Vandesompele et al, 2002).

qPCR data analysis: Target gene qPCR data were normalized to endogenous controls and relative mRNA expression was calculated with the ΔΔCt method through the SDS 2.2/RQ Manager software (Applied Biosystems, Foster City, Calif.) and using the pooled normal samples as the calibrator (Livak and Schmittgen, 2001). Significant differences at p<0.05 in gene expression were determined with the two-tailed, nonparametric Mann-Whitney test for unpaired data using the GraphPad Instat software (Instant Statistics, GraphPad Software, San Diego, Calif.).

### Results

Microarray analysis: From 54,675 genes on the gene chip, through a series of filters, 143 with Genbank assignments were found to be significantly up-regulated (p<0.05) in at least 6 of 10 CIDP samples with a fold change (FC) of >1.5 relative to both CMT1 and normal samples. Similarly, 145 genes were identified as down-regulated in CIDP. Gene ontology, pathway and network analyses were applied to these genes to identify associated functions, processes and diseases.

Of the 143 over-expressed genes in CIDP, only 134 and 91 are in the GO and Ingenuity databases, respectively, at this time. Among the under-expressed genes, 113 and 84 are currently annotated.

The top biological or disease processes most significantly associated with up-regulated genes in CIDP include immunological (27% of genes, p=0.0001-0.0189), cell death (24%, p=0.0001-0.0189), cell signaling/interaction (30%, p=0.0002-0.0189), cellular movement (33%, p=0.0005-0.0189), cancer (32%, p=0.0006-0.0189), inflammatory (27%, p=0.0011-0.0126), and skeletal/muscular system development/function (30%, p=0.0063-0.0126). As many genes have multiple functions across different categories, these groups are not exclusionary.

The processes most significantly associated with down-regulated genes in CIDP include cell growth/proliferation (27% of genes, 0.0001-0.04), cell death (39%, p=0.0001-0.05), small molecule biochemistry (34%, 0.0010-0.05), cancer (44%, 0.0052-0.05), gene expression (23%, p=0.0052-0.05), and neurological (21%, 0.0052-0.05).

The microarray study revealed primarily small changes in gene expression in the skin samples, with fold change values predominantly in the 1.5-3 range, with none above 5. A number of potentially relevant genes, for which Taqman primer-probe sets were available, with the highest fold change from normal or CMT1 were selected for validation by qPCR in addition to a panel of 10 genes that were found to be up-regulated in CIDP sural nerve (Renaud et al, 2005).

Validation of endogenous control genes for qPCR: The high sensitivity of qPCR for assay of gene expression requires one or more stably expressed controls in a given set of tissues for optimal normalization, with housekeeping genes such as GAPDH (glyceraldehyde-3-phosphate dehydrogenase), B-Actin, and 18S ribosomal RNA, which are frequently used for this purpose. However, housekeeping gene expression has been reported to vary considerably and could result in variable target gene expression when used for normalization. Therefore, 10 commonly used housekeeping genes were evaluated for use as endogenous reference controls for skin samples after first determining from the microarray studies that none showed significant expression above normal levels in the neuropathy patients. These genes, GAPDH, B-Actin, 18S, HMBS, HPRT, PGK1, STST1, TBP, and UBC, were assayed for expression in 32 skin biopsies from normal and patient forearm, thigh or finger. Except for GAPDH and PGK1, which are both in the glycolysis pathway, these genes are not co-regulated.

From the qPCR Ct values, the cycle number at which fluorescence crosses a threshold point, the geNorm program calculates the gene expression stability measure, M, for a reference gene as the average pairwise variation for that gene with all other tested reference genes. Stepwise exclusion of the gene with the highest M value allows ranking of the tested genes according to their expression stability (Vandesompele et al, 2002). By this measure, GAPDH and PPIA (peptidylprolyl isomerase A (cyclophilin A)) showed the least variability for skin tissue, with similar low M values of 0.0777. As there was no advantage to using the averaged Ct values of both genes, and normalization with GAPDH yielded fold change values closest to those determined by microarray, GAPDH was selected as the endogenous control for the skin biopsy qPCR studies.

Validation of microarray results by qPCR: Of the panel of 10 genes with elevated expression in CIDP sural nerve only three, AIF1, LYVE-1, and FYB, were also significantly up-regulated in CIDP skin biopsies. An additional two genes of interest with elevated expression in CIDP, MLLT3 and P2RY1 were identified from the microarray studies of skin biopsies. The microarray results for these five genes were confirmed by qPCR (Table 3). The myelin protein, P0, was detected in all samples, indicating that myelinated nerve tissue was present in the skin biopsies, although expression was generally lower among both patient groups compared to normals, probably reflecting a decrease of myelinated fibers in the neuropathy patients.

TABLE 3
Validation of Microarray Results by qPCR
Fold Change (FC)^{a}

| | | | | | | | CIDP vs CMT1 |
|---|---|---|---|---|---|---|---|
| | CIDP vs N | | CMT1 vs N | | CIDP vs CMT1 | | (qPCR) |
| | microarray | qPCR | microarray | qPCR | microarray | qPCR | p value |
| AIF1 | 2.26 ± 1.17 | 6.27 + 7.37 | 1.41 ± 0.17 | 1.13 ± 0.71 | 1.60 | 5.55 | 0.0008 |
| LYVE1 | 2.01 ± 1.04 | 4.20 + 4.91 | 1.32 ± 0.33 | 0.86 ± 0.17 | 1.52 | 4.88 | 0.0007 |
| FYB | 2.50 ± 0.96 | 3.91 + 4.41 | 1.67 ± 0.41 | 1.19 ± 1.21 | 1.50 | 3.29 | 0.0328 |
| MLLT3 | 2.18 ± 0.89 | 2.64 + 2.14 | 1.07 ± 0.45 | 0.60 ± 0.45 | 2.04 | 4.40 | 0.0105 |
| P2RY1 | 1.85 ± 1.28 | 3.81 + 4.06 | 0.71 ± 0.36 | 1.06 ± 0.96 | 2.61 | 3.59 | 0.0203 |

| | | | |
|---|---|---|---|
| Average Index | 20.84 + 21.52 | 4.84 ± 3.03 | 0.0018 |
| (sum of all 5 genes) | | | |

^{a}Fold change ± standard deviation. For microarray, n = 10, 6 and 5 samples for CIDP, CMT1 and normal, respectively. Similarly, for qPCR, n = 11, 8, and 7.

The number of CIDP patients with fold change (FC)>1.5 over normal varied with each of the 5 up-regulated genes, from 8-10 of 11 samples, as compared to 0-2 of 8 CMT1 samples. While the individual gene expression remain significantly different between CIDP and CMT1, a greater differential may be seen with the sum of the FC values for all 5 genes for each patient. This index ranges from 3.37-80.47 among 11 CIDP patients tested by qPCR, and from 1.24-10.25 among 8 CMT1 patients, with the difference between groups statistically significant at p=0.0018.

### Discussion

Further data indicates that each of the up-regulated genes in CIDP may be involved, directly or indirectly, in inflammatory, immune or defense processes. AIF1 is produced by activated macrophages in transplant rejection and autoimmune disorders (Liu et al, 2007; Orsmark et al, 2007), and LYVE-1 is a marker for lymphatic endothelium which is also expressed by activated bone marrow and tissue macrophages (Schledzewski et al, 2006). FYB, also called ADAP (Adhesion and Degranulation Promoting Adaptor Protein), mediates signaling from T-cell antigen receptors to integrins, leading to enhanced cellular adhesion (Peterson 2003). Their increased expression in skin biopsies of patients with CIDP could reflect the presence of terminal nerve fibers, including from myelinated nerves, in skin (Provitera et al, 2007), or result from a systemic inflammatory reaction. P2RY1 is a member of a family of G protein-coupled receptors that are expressed on monocytes and macrophages and are involved in inflammatory and immunity pathways (Pattin et al, 2008). MLLT3 is known to regulate erythrocyte and megakaryocyte differentiation (Pina et al, 2008) and is also induced by ligation of CD44, a cell surface receptor for hyaluronan (HA) closely related in function to LYVE-1 which also binds HA (Hogerkorp et al, 2003). HA levels are elevated in inflammatory and immune responses. Mutation of the MLLT3 gene has also been linked to a patient with neuromotor development delay, cerebellar and epilepsy (Pramparo et al, 2005). It is not yet clear what role it may play in CIDP or other neuropathies. Expression of these 5 markers appear to be unrelated to demyelination, as it is not increased in skin biopsies from patients with hereditary demyelinating neuropathies caused by genetic defects.

The degree of CIDP associated change in expression in the microarray assays for all identified genes was generally low for the skin biopsies and in a range that is often difficult to predict statistically significant confirmation by qPCR. The fold change values were below 5 and predominantly in the 1.5-3 range. However, it is clear that abnormal regulation of some potentially significant genes are verifiable by qPCR. Many of the genes from the skin microarray studies remain to be confirmed and examined for their relationship to CIDP pathogenesis.

The diagnosis of CIDP is currently based on the clinical presentation and results of electrodiagnostic studies. A nerve biopsy, however, may be required to confirm the diagnosis in atypical cases, and CIDP can sometimes occur in patients with otherwise typical diabetic or hereditary neuropathy (Haq et al, 2003; Ginsberg et al, 2004). Although elevations of AIF1, LYVE-1, FYB, MLLT3 and P2RY1 in skin are not specific for CIDP or inflammatory neuropathy, and are likely to be increased in inflammatory conditions that involve the skin, determination of mRNA levels in skin biopsies may help distinguish patients with possible CIDP, from those with non-inflammatory neuropathy such as CMT1. Further studies are needed to determine the usefulness of these markers in the evaluation of patients with disorders of the peripheral nerves.

### REFERENCES

Applied Biosystems (appliedbiosystems.com). Guide to performing relative quantification of gene expression using real-time quantitative PCR, 2004, pp. 47-50.
Renaud S, Hays A P, Brannagan T H, Sander H W, Edgar M, Weimer L H, Olarte M R, Dalakas M C, Xiang Z, Danon M J, Latov N. Gene expression profiling in chronic demyelinating polyneuropathy. J Neuroimmunol 2005, 159: 203-214.
Ginsberg L, Malik O, Kenton A R, Sharp D, Muddle J R, Davis M D, Winer J B, Orrell R W, King R H. Coexistent hereditary and inflammatory neuropathy. Brain 2004; 127: 193-202.
Haq R U, Pendlebury W W, Fries T J, Tanden R. Chronic inflammatory demyelinating polyradiculpathy in diabetic patients. Muscle Nerve 2003; 27: 465-70.
Joint Task Foce of the EFNS and the PNS. European Federation of Neurologic Societies/Peripheral Nerve Society Guidelines on management of chronic inflammatory demyelinating polyradiculoneuropathy. Report of a joint task force of the European Federation of Neurologic Societies and the Peripheral Nerve Society. J Periph Nery Syst 2005; 10: 220-8.
Koller H, Kieseier B C, Jander S, Hartung H P. Chronic inflammatory demyelinating polyneuropathy. N Engl J Med. 2005; 352: 1343-56.
Livak K J, Schmittgen T D. Analysis of relative gene expression data using real-time quantitative PCR and the 2^{-ΔΔCt} method. Methods 2001; 25: 402-408.
Liu G, Ma H, Jiang L, Zhao Y. Allograft inflammatory factor-1 and its immune regulation. Autoimmunity 2007; 40: 95-102.
Malik R A, Tesfaye S, Newrick P G, Walker D, Rabjbhandari S M, Siddique I, Sharma A K, Boulton A J, King R H, Thomas P K, Ward J D. Sural nerve pathology in diabetic patients with minimal but progressive neuropathy. Diabetologia 2005; 48: 578-85.
Orsmark C, Skoog T, Jeskanen L, Kere J, Saarialho-Kere U. Expression of allograft inflammatory factor-1 in inflammatory skin disorders. Acta Derm Venereol 2007; 87: 223-7.
Peterson E J. The TCR ADAPts to integrin mediated cell adhesion. Immunol Rev 2003; 192: 113-121.
Provitera V, Nolano M, Pagano A, Caporaso G, Stancanelli A, Santoro L. Myelinated nerve endings in human skin. Muscle Nerve 2007; 35: 767-775.
Renaud S, Hays A P, Brannagan T H III, Sander H W, Edgar M, Weimer L H, Olarte M R, Dalakas M C, Xiang Z, Danon M J, Latov N. Gene expression profiling in chronic inflammatory demyelinating polyneuropathy. J Neuroimmunol 2005; 159: 203-14.
Rosoklija G B, Dwork A J, Younger D S, Karlikaya G, Latov N, Hays A P. Local activation of the complement system in endoneurial microvessels of diabetic neuropathy. Acta Neuropathol 200; 99: 55-62.
Schledzewski K, Falkowski M, Moldenhauer G, Metharom P, Kzhyshkowska J, Ganss R, Demory A, Falkowska-Hansen B, Kurzen H, Ugurel S, Geginat G, Arnold B, Goerdt S. Lymphatic endothelium-specific hyaluronan receptor LYVE-1 is expressed by stabilin-1+, F4/80+, CD11b+ macrophages in malignant tumours and wound healing tissue in vivo and in bone marrow cultures in vitro: implications for the assessment of lymph-angiogenesis. J Pathol 2006; 209: 67-77.
Shy M E, Garberri J Y, Kamholz J. Hereditary motor and sensory neuropathy; a biological perspective. Lancet Neurol 2002; 110-8.
Younger D S, Rosoklija G, Hays A P, Trojaborg W, Latov N. Diabetic peripheral neuropathy: a clinicopathological and immunohistochemical analysis of sural nerve biopsies. Muscle Nerve 1996; 19: 722-7.
Hogerkorp A M, Bilke S, Breslin T, Ingarsson S, Borrebaeck C A. CD44-stimulated human B cells express transcripts specifically involved in immunomodulation and inflammation as analyzed by DNA microarrays. Blood. 2003; 101:2307-2313
Joint Task Force of the EFNS and the PNS. European Federation of Neurologic Societies/Peripheral Nerve Society Guidelines on management of chronic inflammatory demyelinating polyradiculoneuropathy. Report of a joint task force of the European Federation of Neurologic Societies and the Peripheral Nerve Society. J Periph Nery Syst 2005; 10: 220-8.
Koller H, Kieseier B C, Jander S, Hartung H P. Chronic inflammatory demyelinating polyneuropathy. N Engl J Med. 2005; 352: 1343-56.
Livak K J, Schmittgen T D. Analysis of relative gene expression data using real-time quantitative PCR and the 2-DDCt method. Methods 2001; 25: 402-408.
Liu G, Ma H, Jiang L, Zhao Y. Allograft inflammatory factor-1 and its immune regulation. Autoimmunity 2007; 40: 95-102.
Orsmark C, Skoog T, Jeskanen L, Kere J, Saarialho-Kere U. Expression of allograft inflammatory factor-1 in inflammatory skin disorders. Acta Derm Venereol 2007; 87: 223-7.
Peterson E J. The TCR ADAPts to integrin mediated cell adhesion. Immunol Rev 2003; 192: 113-121.
Pina C, May G, Soneji S, Hong D, Enver T. MLLT3 regulates early human erythroid and megakaryocytic cell fate. Cell Stem Cell 2008 Mar. 6; 2(3):264-73.
Pramparo T, Grosso S, Messa J, Zatterale A, Bonaglia M C, Chessa L, Balestri P, Rocchi M, Zuffardi O, Giorda R. Loss-of-function mutation of the A F9/MLLT3 gene in a girl with neuromotor development delay, cerebellar ataxia, and epilepsy. Hum Genet. 2005 October; 118(1):76-81.
Lattin J E, Schroder K, Su A I, Walker J R, Zhang J, Wiltshire T, Saijo K, Glass C K, Hume D A, Kellie S, Sweet M J. Expression analysis of G Protein-Coupled Receptors in mouse macrophages. Immunome Res. 2008 Apr. 29; 4(1):5.
Provitera V, Nolano M, Pagano A, Caporaso G, Stancanelli A, Santoro L. Myelinated nerve endings in human skin. Muscle Nerve 2007; 35: 767-775.
Renaud S, Hays A P, Brannagan T H III, Sander H W, Edgar M, Weimer L H, Olarte M R, Dalakas M C, Xiang Z, Danon M J, Latov N. Gene expression profiling in chronic inflammatory delyelinating polyeneuropathy. J Neuroimmunol 2005; 159: 203-14.
Schledzewski K, Falkowski M, Moldenhauer G, Metharom P, Kzhyshkowska J, Ganss R, Demory A, Falkowska-Hansen B, Kurzen H, Ugurel S, Geginat G, Arnold B, Goerdt S. Lymphatic endothelium-specific hyaluronan receptor LYVE-1 is expressed by stabilin-1+, F4/80+, CD11b+ macrophages in malignant tumours and wound healing tissue in vivo and in bone marrow cultures in vitro: implications for the assessment of lymphangiogenesis. J Pathol 2006; 209: 67-77.
Shy M E, Garberri J Y, Kamholz J. Hereditary motor and sensory neuropathy; a biological perspective. Lancet Neurol 2002; 110-8.
Vandesompele J, De Preter K, Pattyn F, Poppe B, Van Roy N, De Paepe A, Speleman F. Accurate normalization of real-time quantitative R T-PCR data by geometric averaging of multiple internal control genes. Genome Biol 2002; 3: research 0034.1-0034.11.

All patents and publications referenced or mentioned herein are indicative of the levels of skill of those skilled in the art to which the invention pertains, and each such referenced patent or publication is hereby incorporated by reference to the same extent as if it had been incorporated by reference in its entirety individually or set forth herein in its entirety. Applicants reserve the right to physically incorporate into this specification any and all materials and information from any such cited patents or publications.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. The methods and processes illustratively described herein suitably may be practiced in differing orders of steps, and that they are not necessarily restricted to the orders of steps indicated herein or in the claims. As used herein and in the appended claims, the singular forms “a,” “an,” and “the” include plural reference unless the context clearly dictates otherwise. Thus, for example, a reference to “an antibody” includes a plurality (for example, a solution of antibodies or a series of antibody preparations) of such antibodies, and so forth. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

What is claimed:

1. A method of detecting or monitoring inflammatory neuropathy in a patient comprising:
a. obtaining a biological sample from the patient;
b. comparing a test expression level of allograft inflammatory factor 1 (AIF1), lymphatic hyaluronan receptor (LYVE-1), FYN binding protein (FYB), myeloid/lymphoid or mixed-lineage leukemia, translocated to, 3 (MLLT3), purinergic receptor P2Y, G-protein coupled, 1 (P2RY1) or a combination thereof in the biological sample with a control expression level of allograft inflammatory factor 1 (AIF1), lymphatic hyaluronan receptor (LYVE-1), FYN binding protein (FYB), myeloid/lymphoid or mixed-lineage leukemia, translocated to, 3 (MLLT3), purinergic receptor P2Y, G-protein coupled, 1 (P2RY1) or a combination thereof; and
c. detecting inflammatory neuropathy when the test expression level is at least 1.5-fold greater than the control expression level.

2. The method of claim 1, wherein the biological sample is a skin biopsy.

3. The method of claim 1, wherein the biological sample is a nerve biopsy.

4. The methods of claim 1, wherein the inflammatory neuropathy is an infectious neuropathy or an autoimmune neuropathy.

5. The method of claim 1, wherein the inflammatory neuropathy comprises Lyme disease, HIV infection, AIDS, Leprosy, Herpes Zoster (Shingles), Hepatitis B infection, Hepatitis C infection, an autoimmune disease, Sarcoidosis, Guillain-Barré Syndrome, Acute Inflammatory Demyelinating Polyneuropathy (AIDP), Chronic Inflammatory Demyelinating Polyneuropathy (CIDP), Vasculitis, Polyarteritis Nodosa (PAN), Rheumatoid Arthritis, Systemic Lupus Erythematosus, Sjögren's Syndrome, Celiac Disease, Multifocal Motor Neuropathy (MNN), Peripheral Neuropathy Associated with Protein Abnormalities, Monoclonal Gammopathy, Amyloidosis, Cryoglobulinemia and/or POEMS) or a combination thereof.

6. The method of claim 1, wherein the inflammatory neuropathy is chronic inflammatory demyelinating polyneuropathy (CIDP).

7. The method of claim 1, wherein the control expression levels are expression levels of allograft inflammatory factor 1 (AIF1), lymphatic hyaluronan receptor (LYVE-1), FYN binding protein (FYB), myeloid/lymphoid or mixed-lineage leukemia, translocated to, 3 (MLLT3), purinergic receptor P2Y, G-protein coupled, 1 (P2RY1) or a combination thereof, in a biological sample from a healthy patient who does not have inflammatory neuropathy.

8. The method of claim 1, wherein the control expression levels are expression levels of allograft inflammatory factor 1 (AIF1), lymphatic hyaluronan receptor (LYVE-1), FYN binding protein (FYB), myeloid/lymphoid or mixed-lineage leukemia, translocated to, 3 (MLLT3), purinergic receptor P2Y, G-protein coupled, 1 (P2RY1), or a combination thereof, in a biological sample from a patient with non-inflammatory neuropathy.

9. The method of claim 1, wherein the control expression levels are expression levels of allograft inflammatory factor 1 (AIF1), lymphatic hyaluronan receptor (LYVE-1), FYN binding protein (FYB), myeloid/lymphoid or mixed-lineage leukemia, translocated to, 3 (MLLT3), purinergic receptor P2Y, G-protein coupled, 1 (P2RY1), or a combination thereof, in a biological sample from a patient with hereditary demyelinating neuropathy, Charcot-Marie-Tooth disease type I (CMT1), or diabetic neuropathy (DN).

10. The method of claim 1, wherein inflammatory neuropathy is detected or diagnosed when the lymphatic hyaluronan receptor (LYVE-1) expression level in the biological sample is about 2 to about 3 fold greater than the control lymphatic hyaluronan receptor (LYVE-1) expression levels.

11. The method of claim 1, wherein inflammatory neuropathy is detected or diagnosed when the allograft inflammatory factor 1 (AIF1) expression level in the biological sample is about 2 to about 8 fold greater than the control allograft inflammatory factor 1 (AIF1) expression levels.

12. The method of claim 1, wherein inflammatory neuropathy is detected or diagnosed when the FYN binding protein (FYB) expression level in the biological sample is about 1.5 to about 3 fold greater than the control FYN binding protein (FYB) expression level.

13. The method of claim 1, wherein inflammatory neuropathy is detected or diagnosed when the purinergic receptor P2Y, G-protein coupled, 1 (P2RY1) expression level in the biological sample is about 1.5 to about 3 fold greater than the control purinergic receptor P2Y, G-protein coupled, 1 (P2RY1) expression level.

14. The method of claim 1, wherein inflammatory neuropathy is detected or diagnosed when the myeloid/lymphoid or mixed-lineage leukemia, translocated to, 3 (MLLT3) expression level in the biological sample is about 1.5 to about 3 fold greater than the control myeloid/lymphoid or mixed-lineage leukemia, translocated to, 3 (MLLT3) expression level.

15. The method of claim 1, wherein test expression levels and control expression levels are determined by a quantitative real time polymerase chain reaction assay.

16. The method claim 15, wherein primers for the quantitative real time polymerase chain reaction assay selectively hybridize to any of SEQ ID NO:1, 3, 5, 7, 9, 11, 13, 14, or a combination thereof, under stringent hybridization conditions.

17. The method claim 1, wherein test expression levels and control expression levels are determined by quantitative RNA hybridization assay.

18. The method claim 17, wherein probes for the quantitative RNA hybridization assay selectively hybridize to any of SEQ ID NO:1, 3, 5, 7, 9, 11, 13, 14, or a combination thereof, under stringent hybridization conditions.

19. The method claim 1, wherein test expression levels and control expression levels are determined by quantitative microarray analysis.

20. The method claim 19, wherein probes for the quantitative microarray analysis selectively hybridize to any of SEQ ID NO:1, 3, 5, 7, 9, 11, 13, 14, or a combination thereof, under stringent hybridization conditions.

21. The method claim 1, wherein test expression levels and control expression levels are determined by quantitative northern hybridization assay.

22. The method claim 21, wherein probes for the quantitative northern hybridization assay selectively hybridize to any of SEQ ID NO:1, 3, 5, 7, 9, 11, 13, 14, or a combination thereof, under stringent hybridization conditions.

23. A method of detecting or monitoring inflammatory neuropathy in a patient comprising:
(a) obtaining a test skin biopsy from a patient;
(b) quantifying expression of lymphatic hyaluronan receptor (LYVE-1), myeloid/lymphoid or mixed-lineage leukemia, translocated to, 3 (MLLT3), purinergic receptor P2Y, G-protein coupled, 1 (P2RY1) or a combination thereof in the test skin biopsy to obtain quantitative test expression levels of lymphatic hyaluronan receptor (LYVE-1), myeloid/lymphoid or mixed-lineage leukemia, translocated to, 3 (MLLT3) and/or purinergic receptor P2Y, G-protein coupled, 1 (P2RY1);
(c) determining whether the quantitative test expression levels are greater than quantitative control expression levels of lymphatic hyaluronan receptor (LYVE-1), myeloid/lymphoid or mixed-lineage leukemia, translocated to, 3 (MLLT3) and/or purinergic receptor P2Y, G-protein coupled, 1 (P2RY1) in a control skin biopsy;
(d) detecting inflammatory neuropathy when the quantitative test expression levels are at least 2-fold greater than the quantitative control expression levels.

24. The method of claim 23, wherein the control skin biopsy is a skin biopsy of a normal patient who does not have inflammatory neuropathy.

25. The method of claim 23, wherein the control skin biopsy is a skin biopsy of a normal patient who does not have inflammatory neuropathy.

26. The method of claim 23, wherein the control skin biopsy is a skin biopsy of a patient with non-inflammatory neuropathy.

27. The method of claim 23, wherein the control skin biopsy is a skin biopsy of a patient with hereditary demyelinating neuropathy, Charcot-Marie-Tooth disease type I (CMT1), or diabetic neuropathy (DN).

28. The method of claim 23, wherein quantifying expression of lymphatic hyaluronan receptor (LYVE-1), myeloid/lymphoid or mixed-lineage leukemia, translocated to, 3 (MLLT3) and/or purinergic receptor P2Y, G-protein coupled, 1 (P2RY1) in the test skin biopsy is performed using probes or primers selected from, or complementary to, a region of any of SEQ ID NO:1, 3, 5, 7, 9, 11, 13, 14, or a combination thereof.

29. The method of claim 28, wherein the probes or primers selectively hybridize to a region of any of SEQ ID NO:1, 3, 5, 7, 9, 11, 13, 14, or a combination thereof, under stringent hybridization conditions.

30. The method of claim 23, wherein the quantitative control expression levels of lymphatic hyaluronan receptor (LYVE-1) in a control skin biopsy are determined using probes or primers selected from, or complementary to, a region of any of SEQ ID NO:1, 3, 5, 7, 9, 11, 13, 14, or a combination thereof.

31. The method of claim 30, wherein the probes or primers selectively hybridize to a region of any of SEQ ID NO:1, 3, 5, 7, 9, 11, 13, 14, or a combination thereof, under stringent hybridization conditions.

32. A method of detecting or monitoring chronic inflammatory demyelinating polyradiculoneuropathy (CIDP) in a patient comprising:
(a) obtaining a skin biopsy from the patient;
(b) comparing a test expression level of allograft inflammatory factor 1 (AIF1), lymphatic hyaluronan receptor (LYVE-1), FYN binding protein (FYB), myeloid/lymphoid or mixed-lineage leukemia, translocated to, 3 (MLLT3), purinergic receptor P2Y, G-protein coupled, 1 (P2RY1) or a combination thereof, in the skin biopsy, with a control expression level of allograft inflammatory factor 1 (AIF1), lymphatic hyaluronan receptor (LYVE-1), FYN binding protein (FYB), myeloid/lymphoid or mixed-lineage leukemia, translocated to, 3 (MLLT3), purinergic receptor P2Y, G-protein coupled, 1 (P2RY1), or a combination thereof, in a control sample from a patient with hereditary demyelinating neuropathy; and
(c) detecting chronic inflammatory demyelinating polyradiculoneuropathy (CIDP) when the test expression level is at least 1.5-fold greater than the control expression level.


SEQUENCE LISTING (16 sequences)

SEQ ID NO: 1 - DNA, 2518 bp, Homo sapiens
ctcatttgtgtgttttctgagtcagcattagctaaattttccagaaggccatccacaaagtacagcctgggcgttcaagggacgtcattcatttcccccagtgaccttgacaagtcagaagcttgaaagcagggaaatccggatgtctcggttatgaagtggagcagtgagtgtgagcctcaacatagttccagaactctccatccggactagttattgagcatctgcctctcatatcaccagtggccatctgaggtgtttccctggctctgaaggggtaggcacgatggccaggtgcttcagcctggtgttgcttctcacttccatctggaccacgaggctcctggtccaaggctctttgcgtgcagaagagctttccatccaggtgtcatgcagaattatggggatcacccttgtgagcaaaaaggcgaaccagcagctgaatttcacagaagctaaggaggcctgtaggctgctgggactaagtttggccggcaaggaccaagttgaaacagccttgaaagctagctttgaaacttgcagctatggctgggttggagatggattcgtggtcatctctaggattagcccaaaccccaagtgtgggaaaaatggggtgggtgtcctgatttggaaggttccagtgagccgacagtttgcagcctattgttacaactcatctgatacttggactaactcgtgcattccagaaattatcaccaccaaagatcccatattcaacactcaaactgcaacacaaacaacagaatttattgtcagtgacagtacctactcggtggcatccccttactctacaatacctgcccctactactactcctcctgctccagcttccacttctattccacggagaaaaaaattgatttgtgtcacagaagtttttatggaaactagcaccatgtctacagaaactgaaccatttgttgaaaataaagcagcattcaagaatgaagctgctgggtttggaggtgtccccacggctctgctagtgcttgctctcctcttctttggtgctgcagctggtcttggattttgctatgtcaaaaggtatgtgaaggccttcccttttacaaacaagaatcagcagaaggaaatgatcgaaaccaaagtagtaaaggaggagaaggccaatgatagcaaccctaatgaggaatcaaagaaaactgataaaaacccagaagagtccaagagtccaagcaaaactaccgtgcgatgcctggaagctgaagtttagatgagacagaaatgaggagacacacctgaggctggtttctttcatgctccttaccctgccccagctggggaaatcaaaagggccaaagaaccaaagaagaaagtccacccttggttcctaactggaatcagctcaggactgccattggactatggagtgcaccaaagagaatgcccttctccttattgtaaccctgtctggatcctatcctcctacctccaaagcttcccacggcctttctagcctggctatgtcctaataatatcccactgggagaaaggagttttgcaaagtgcaaggacctaaaacatctcatcagtatccagtggtaaaaaggcctcctggctgtctgaggctaggtgggttgaaagccaaggagtcactgagaccaaggctttctctactgattccgcagctcagaccctttcttcagctctgaaagagaaacacgtatcccacctgacatgtccttctgagcccggtaagagcaaaagaatggcagaaaagtttagcccctgaaagccatggagattctcataacttgagacctaatctctgtaaagctaaaataaagaaatagaacaaggctgaggatacgacagtacactgtcagcagggactgtaaacacagacagggtcaaagtgttttctctgaacacattgagttggaatcactgtttagaacacacacacttactttttctggtctctaccactgctgatattttctctaggaaatatacttttacaagtaacaaaaataaaaactcttataaatttctatttttatctgagttacagaaatgattactaaggaagattactcagtaatttgtttaaaaagtaataaaattcaacaaacatttgctgaatagctactatatgtcaagtgctgtgcaaggtattacactctgtaattgaatattattcctcaaaaaattgcacatagtagaacgctatctgggaagctatttttttcagttttgatatttctagcttatctacttccaaactaatttttatttttgctgagactaatcttattcattttctctaatatggcaaccattataaccttaatttattattaacatacctaagaagtacattgttacctctatataccaaagcacattttaaaagtgccattaacaaatgtatcactagccctcctttttccaacaagaagggactgagagatgcagaaatatttgtgacaaaaaattaaagcatttagaaaacttcaaaaaaaaaaaaaaaaaa

SEQ ID NO: 2 - protein, 322 aa, Homo sapiens
MARCFSLVLLLTSIWTTRLLVQGSLRAEELSIQVSCRIMGITLVSKKANQQLNFTEAKEACRLLGLSLAGKDQVETALKASFETCSYGWVGDGFVVISRISPNPKCGKNGVGVLIWKVPVSRQFAAYCYNSSDTWTNSCIPEIITTKDPIFNTQTATQTTEFIVSDSTYSVASPYSTIPAPTTTPPAPASTSIPRRKKLICVTEVFMETSTMSTETEPFVENKAAFKNEAAGFGGVPTALLVLALLFFGAAAGLGFCYVKRYVKAFPFTNKNQQKEMIETKVVKEEKANDSNPNEESKKTDKNPEESKSPSKTTVRCLEAEV

SEQ ID NO: 3 - DNA, 2282 bp, Homo sapiens
agtggccatctgaggtgtttccctggctctgaaggggtaggcacgatggccaggtgcttcagcctggtgttgcttctcacttccatctggaccacgaggctcctggtccaaggctctttgcgtgcagaagagctttccatccaggtgtcatgcagaattatggggatcacccttgtgagcaaaaaggcgaaccagcagctgaatttcacagaagctaaggaggcctgtaggctgctgggactaagtttggccggcaaggaccaagttgaaacagccttgaaagctagctttgaaacttgcagctatggctgggttggagatggattcgtggtcatctctaggattagcccaaaccccaagtgtgggaaaaatggggtgggtgtcctgattaggaaggttccagtgagccgacagtttgcagcctattgttacaactcatctgatacttggactaactcgtgcattccagaaattatcaccaccaaagatcccatattcaacactcaaactgcaacacaaacaacagaatttattgtcagtgacagtacctactcggtggcatccccttactctacaatacctgcccctactactactcctcctgctccagcttccacttctattccacggagaaaaaaattgatttgtgtcacagaagtttttatggaaactagcaccatgtctacagaaactgaaccatttgttgaaaataaagcagcattcaagaatgaagctgctgggtttggaggtgtccccacggctctgctagtgcttgctctcctcttctttggtgctgcagctggtcttggattttgctatgtcaaaaggtatgtgaaggccttcccttttacaaacaagaatcagcagaaggaaatgatcgaaaccaaagtagtaaaggaggagaaggccaatgatagcaaccctaatgaggaatcaaagaaaactgataaaaacccagaagagtccaagagtccaagcaaaactaccgtgcgatgcctggaagctgaagtttagatgagacagaaatgaggagacacacctgaggctggtttctttcatgctccttaccctgccccagctggggaaatcaaaagggccaaagaaccaaagaagaaagtccacccttggttcctaactggaatcagctcaggactgccattggactatggagtgcaccaaagagaatgcccttctccttattgtaaccctgtctggatcctatcctcctacctccaaagcttcccacggcctttctagcctggctatgtcctaataatatcccactgggagaaaggagttttgcaaagtgcaaggacctaaaacatctcatcagtatccagtggtaaaaaggcctcctggctgtctgaggctaggtgggttgaaagccaaggagtcactgagaccaaggctttctctactgattccgcagctcagaccctttcttcagctctgaaagagaaacacgtatcccacctgacatgtccttctgagcccggtaagagcaaaagaatggcagaaaagtttagcccctgaaagccatggagattctcataacttgagacctaatctctgtaaagctaaaataaagaaatagaacaaggctgaggatacgacagtacactgtcagcagggactgtaaacacagacagggtcaaagtgttttctctgaacacattgagttggaatcactgtttagaacacacacacttactttttctggtctctaccactgctgatattttctctaggaaatatacttttacaagtaacaaaaataaaaactcttataaatttctatttttatctgagttacagaagtgattactaaggaagattactcagtaatttgtttaaaaagtaataaaattcaacaaacatttgctgaatagctactatatgtcaagtgctgtgcaaggtattacactctgtaattgaatattattcctcaaaaaattgcacatagtagaacgctatctgggaagctgtttttttcagttttgatatttctagcttatctacttccaaactaatttttgtttttactgagactaatcttattcattttctctaatatggcaaccattataaccttaatttattattaacatacctaagaagtacattgttacctctatataccaaagcacattttaaaagtgccattaacaaatgtatcactagccctcctttttccaacaagaagggactgagagatgcagaaatatttgtgacaaaaaattaaagcatttaggaaaaaaaaaaaaaaaaaaaaaaaaaaaaaa

SEQ ID NO: 4 - protein, 322 aa, Homo sapiens
MARCFSLVLLLTSIWTTRLLVQGSLRAEELSIQVSCRIMGITLVSKKANQQLNFTEAKEACRLLGLSLAGKDQVETALKASFETCSYGWVGDGFVVISRISPNPKCGKNGVGVLIRKVPVSRQFAAYCYNSSDTWTNSCIPEIITTKDPIFNTQTATQTTEFIVSDSTYSVASPYSTIPAPTTTPPAPASTSIPRRKKLICVTEVFMETSTMSTETEPFVENKAAFKNEAAGFGGVPTALLVLALLFFGAAAGLGFCYVKRYVKAFPFTNKNQQKEMIETKVVKEEKANDSNPNEESKKTDKNPEESKSPSKTTVRCLEAEV

SEQ ID NO: 5 - DNA, 503 bp, Homo sapiens
cacctagcagttggttggcaaccccttcctcagtcccctgctgaaaaccctccagtcagcgcttatcccttctgctctctcccctcacccagagaaatacatggagtttgaccttaatggaaatggcgatattgatatcatgtccctgaaacgaatgctggagaaacttggagtccccaagactcacctagagctaaagaaattaattggagaggtgtccagtggctccggggagacgttcagctaccctgactttctcaggatgatgctgggcaagagatctgccatcctaaaaatgatcctgatgtatgaggaaaaagcgagagaaaaggaaaagccaacaggccccccagccaagaaagctatctctgagttgccctgatttgaagggaaaagggatgatgggattgaaggggcttctaatgacccagatatggaaacagaagacaaaattgtaagccagagtcaacaaattaaataaattaccccctcctccagatcaa

SEQ ID NO: 6 - protein, 93 aa, Homo sapiens
MEFDLNGNGDIDIMSLKRMLEKLGVPKTHLELKKLIGEVSSGSGETFSYPDFLRMMLGKRSAILKMILMYEEKAREKEKPTGPPAKKAISELP

SEQ ID NO: 7 - DNA, 639 bp, Homo sapiens
gagagaaggagagcctgcagacagaggcctccagcttggtctgtctccccacctctaccagcatctgctgagctatgagccaaaccagggatttacagggaggaaaagctttcggactgctgaaggcccagcaggaagagaggctggatgagatcaacaagcaattcctagacgatcccaaatatagcagtgatgaggatctgccctccaaactggaaggcttcaaagagaaatacatggagtttgaccttaatggaaatggcgatattgatatcatgtccctgaaacgaatgctggagaaacttggagtccccaagactcacctagagctaaagaaattaattggagaggtgtccagtggctccggggagacgttcagctaccctgactttctcaggatgatgctgggcaagagatctgccatcctaaaaatgatcctgatgtatgaggaaaaagcgagagaaaaggaaaagccaacaggccccccagccaagaaagctatctctgagttgccctgatttgaagggaaaagggatgatgggattgaaggggcttctaatgacccagatatggaaacagaagacaaaattgtaagccagagtcaacaaattaaataaattaccccctcctccagatcaa

SEQ ID NO: 8 - protein, 147 aa, Homo sapiens
MSQTRDLQGGKAFGLLKAQQEERLDEINKQFLDDPKYSSDEDLPSKLEGFKEKYMEFDLNGNGDIDIMSLKRMLEKLGVPKTHLELKKLIGEVSSGSGETFSYPDFLRMMLGKRSAILKMILMYEEKAREKEKPTGPPAKKAISELP

SEQ ID NO: 9 - DNA, 4876 bp, Homo sapiens
ccgcagttcttgagttccacatgcagagcagatgcgacagctagaagtgagtagggcccagaccctggcccaggaagatccactaaaggaggccatccttccgccttcttctgcaggagtcaggatggaaaggcagatgtaaagtccctcatggcgaaatataacacggggggcaacccgacagaggatgtctcagtcaatagccgacccttcagagtcacagggccaaactcatcttcaggaatacaagcaagaaagaacttattcaacaaccaaggaaatgccagccctcctgcaggacccagcaatgtacctaagtttgggtccccaaagccacctgtggcagtcaaaccttcttctgaggaaaagcctgacaaggaacccaagcccccgtttctaaagcccactggagcaggccaaagattcggaacaccagccagcttgaccaccagagaccccgaggcgaaagtgggatttctgaaacctgtaggccccaagcccatcaacttgcccaaagaagattccaaacctacatttccctggcctcctggaaacaagccatctcttcacagtgtaaaccaagaccatgacttaaagccactaggcccgaaatctgggcctactcctccaacctcagaaaatgaacagaagcaagcgtttcccaaattgactggggttaaagggaaatttatgtcagcatcacaagatcttgaacccaagcccctcttccccaaacccgcctttggccagaagccgcccctaagtaccgagaactcccatgaagacgaaagccccatgaagaatgtgtcttcatcaaaagggtccccagctcccctgggagtcaggtccaaaagcggccctttaaaaccagcaagggaagactcagaaaataaagaccatgcaggggagatttcaagtttgccctttcctggagtggttttgaaacctgctgcgagcaggggaggcccaggtctctccaaaaatggtgaagaaaaaaaggaagataggaagatagatgctgctaagaacaccttccagagcaaaataaatcaggaagagttggcctcagggactcctcctgccaggttccctaaggccccttctaagctgacagtgggggggccatggggccaaagtcaggaaaaggaaaagggagacaagaattcagccaccccgaaacagaagccattgcctcccttgtttaccttgggtccacctccaccaaaacccaacagaccaccaaatgttgacctgacgaaattccacaaaacctcttctggaaacagtactagcaaaggccagacgtcttactcaacaacttccctgccaccacctccaccatcccatccggccagccaaccaccattgccagcatctcacccatcacaaccaccagtcccaagcctacctcccagaaacattaaacctccgtttgacctaaaaagccctgtcaatgaagacaatcaagatggtgtcacgcactctgatggtgctggaaatctagatgaggaacaagacagtgaaggagaaacatatgaagacatagaagcatccaaagaaagagagaagaaaagggaaaaggaagaaaagaagaggttagagctggagaaaaaggaacagaaagagaaagaaaagaaagaacaagaaataaagaagaaatttaaactaacaggccctattcaagtcatccatcttgcaaaagcttgttgtgatgtcaaaggaggaaagaatgaactgagcttcaagcaaggagagcaaattgaaatcatccgcatcacagacaacccagaaggaaaatggttgggcagaacagcaaggggttcatatggctatattaaaacaactgctgtagagattgactatgattctttgaaactgaaaaaagactctcttggtgccccttcaagacctattgaagatgaccaagaagtatatgatgatgttgcagagcaggatgatattagcagccacagtcagagtggaagtggagggatattccctccaccaccagatgatgacatttatgatgggattgaagaggaagatgctgatgatggctccacactacaggttcaagagaagagtaatacgtggtcctgggggattttgaagatgttaaagggaaaagatgacagaaagaaaagtatacgagagaaacctaaagtctctgactcagacaataatgaaggttcatctttccctgctcctcctaaacaattggacatgggagatgaagtttacgatgatgtggatacctctgatttccctgtttcatcagcagagatgagtcaaggaactaatgttggaaaagctaagacagaagaaaaggaccttaagaagctaaaaaagcaggaaaaagaagaaaaagacttcaggaaaaaatttaaatatgatggtgaaattagagtcctatattcaactaaagttacaacttccataacttctaaaaagtggggaaccagagatctacaggtaaaacctggtgaatctctagaagttatacaaaccacagatgacacaaaagttctctgcagaaatgaagaagggaaatatggttatgtccttcggagttacctagcggacaatgatggagagatctatgatgatattgctgatggctgcatctatgacaatgactagcactcaactttggtcattctgctgtgttcattaggtgccaatgtgaagtctggattttaattggcatgttattgggtatcaagaaaattaatgcacaaaaccacttattatcatttgttatgaaatcccaattatctttacaaagtgtttaaagtttgaacatagaaaataatctctctgcttaattgttaactcagaagactacattagtgagatgtaagaattattaaatattccatttccgctttggctacaattatgaagaagttgaaggtacttcttttagaccaccagtaaataatcctccttcaaaaaataaaaataaaagaaaaaggaaaatcattcaggaagaaatgacctgtctaaaaaaacctaaggaagaataataatataagaaaggaaatttaaaaacattccacaagaagaaaaattattgtttatacttctacttatggttatatcttatattctctattcaagtgacctgtcttttaaaaaggcagtgctgtcttacctcttgctagtgggttaaatgttttcaaaaattatagcagtagtagaagttttgtataaaatttgtccttatttgttaattgtatataaatgttaattatttgatacgaatgttatgcatttagtatgcacattgaagtctaaactgtagaagagtctaaaacaagttctctttttgcagattcacatactaatggtttaattctgtgctctgtttaaagtactattataactagagtagatctgaatgaggataaccctaaaatcatgaggaatggaagaatggaccttgaaactacctaggcttttatgcatggcacctctttataatgaagacactttttaaagtttttgtttttgtttcaattaccgctagatttttttttctctttttttaaaatccattttactggaaagttggccagcagagggagtagaaattattaaaattctagtgtttggattgggcccttctctaacagtacatactcattcccaaagcaatccaaaaacaaaatgtgaaccatttgggtttcaaatgttaagaacactaaatagcatgatttaaaaaatgaaaaatgctaacacccaagaaaagaagatattaagtgctttttaacaactcctagagtacaaaatgagtacatcataatgctggctcttctactaatgaaccatcgagtgatattgaataaattatttatcttctcagtttccttatctgtaaattacaatattagactaagtaagtttttccaactcttcactaccaattaccttaggcttttataatgctccgcctacttcagtcccatgtttcagaagcttttgtctattttttaaactcattgattaaataatgattaatgcattctccacattttaatattgcaaaggcccattggagtttctgaagtggctccacagaattgaaataatttcaaataactgtaaaggaactgaaaatcttcacagagatgaagtggggtttccattaggtgctttgaaatttgataacaaatcatcaacttccactggtcaatatatagattttgggtgtctgaggccccaagattagatgccactaatctccaaagattccctccaattatgaaatattttaatgtctacttttagagagcactagccagtatatgaccatgtgattaatttcttttcacactagataaaattacctggttcaaaagtggtttttgtttattaaatttggtaataaatatatataatacacagacaggatagtttttatgctgaagtttttggccagctttagtttgaggactccttgataagcttgctaaactttcagagtgccctgagacacttccagccatccctcctcctgccttcattggggcagacttgcattgcagtctgacagtaattttttttctgattgagaattatgtaaattcaatacaatgtcagtttttaaaagtcaaagttagatcaagagaatatttcagagttttggtttacacatcaagaaacagacacacatacctaggaaagatttacacaatagataatcatcttaatgtgaaagatatttgaagtattaattttaatatattaaatatgatttctgttatagtcttctgtatggaattttgtcacttaagatgagctgcaaataaataataccttcaatggaaaaaaaaaaaaaaaaaaaaaaaaaaaaaaaaaaaaaaaaaaaaaaaaa

SEQ ID NO: 10 - protein, 829 aa, Homo sapiens
MAKYNTGGNPTEDVSVNSRPFRVTGPNSSSGIQARKNLFNNQGNASPPAGPSNVPKFGSPKPPVAVKPSSEEKPDKEPKPPFLKPTGAGQRFGTPASLTTRDPEAKVGFLKPVGPKPINLPKEDSKPTFPWPPGNKPSLHSVNQDHDLKPLGPKSGPTPPTSENEQKQAFPKLTGVKGKFMSASQDLEPKPLFPKPAFGQKPPLSTENSHEDESPMKNVSSSKGSPAPLGVRSKSGPLKPAREDSENKDHAGEISSLPFPGVVLKPAASRGGPGLSKNGEEKKEDRKIDAAKNTFQSKINQEELASGTPPARFPKAPSKLTVGGPWGQSQEKEKGDKNSATPKQKPLPPLFTLGPPPPKPNRPPNVDLTKFHKTSSGNSTSKGQTSYSTTSLPPPPPSHPASQPPLPASHPSQPPVPSLPPRNIKPPFDLKSPVNEDNQDGVTHSDGAGNLDEEQDSEGETYEDIEASKEREKKREKEEKKRLELEKKEQKEKEKKEQEIKKKFKLTGPIQVIHLAKACCDVKGGKNELSFKQGEQIEIIRITDNPEGKWLGRTARGSYGYIKTTAVEIDYDSLKLKKDSLGAPSRPIEDDQEVYDDVAEQDDISSHSQSGSGGIFPPPPDDDIYDGIEEEDADDGSTLQVQEKSNTWSWGILKMLKGKDDRKKSIREKPKVSDSDNNEGSSFPAPPKQLDMGDEVYDDVDTSDFPVSSAEMSQGTNVGKAKTEEKDLKKLKKQEKEEKDFRKKFKYDGEIRVLYSTKVTTSITSKKWGTRDLQVKPGESLEVIQTTDDTKVLCRNEEGKYGYVLRSYLADNDGEIYDDIADGCIYDND

SEQ ID NO: 11 - DNA, 4738 bp, Homo sapiens
ccgcagttcttgagttccacatgcagagcagatgcgacagctagaagtgagtagggcccagaccctggcccaggaagatccactaaaggaggccatccttccgccttcttctgcaggagtcaggatggaaaggcagatgtaaagtccctcatggcgaaatataacacggggggcaacccgacagaggatgtctcagtcaatagccgacccttcagagtcacagggccaaactcatcttcaggaatacaagcaagaaagaacttattcaacaaccaaggaaatgccagccctcctgcaggacccagcaatgtacctaagtttgggtccccaaagccacctgtggcagtcaaaccttcttctgaggaaaagcctgacaaggaacccaagcccccgtttctaaagcccactggagcaggccaaagattcggaacaccagccagcttgaccaccagagaccccgaggcgaaagtgggatttctgaaacctgtaggccccaagcccatcaacttgcccaaagaagattccaaacctacatttccctggcctcctggaaacaagccatctcttcacagtgtaaaccaagaccatgacttaaagccactaggcccgaaatctgggcctactcctccaacctcagaaaatgaacagaagcaagcgtttcccaaattgactggggttaaagggaaatttatgtcagcatcacaagatcttgaacccaagcccctcttccccaaacccgcctttggccagaagccgcccctaagtaccgagaactcccatgaagacgaaagccccatgaagaatgtgtcttcatcaaaagggtccccagctcccctgggagtcaggtccaaaagcggccctttaaaaccagcaagggaagactcagaaaataaagaccatgcaggggagatttcaagtttgccctttcctggagtggttttgaaacctgctgcgagcaggggaggcccaggtctctccaaaaatggtgaagaaaaaaaggaagataggaagatagatgctgctaagaacaccttccagagcaaaataaatcaggaagagttggcctcagggactcctcctgccaggttccctaaggccccttctaagctgacagtgggggggccatggggccaaagtcaggaaaaggaaaagggagacaagaattcagccaccccgaaacagaagccattgcctcccttgtttaccttgggtccacctccaccaaaacccaacagaccaccaaatgttgacctgacgaaattccacaaaacctcttctggaaacagtactagcaaaggccagacgtcttactcaacaacttccctgccaccacctccaccatcccatccggccagccaaccaccattgccagcatctcacccatcacaaccaccagtcccaagcctacctcccagaaacattaaacctccgtttgacctaaaaagccctgtcaatgaagacaatcaagatggtgtcacgcactctgatggtgctggaaatctagatgaggaacaagacagtgaaggagaaacatatgaagacatagaagcatccaaagaaagagagaagaaaagggaaaaggaagaaaagaagaggttagagctggagaaaaaggaacagaaagagaaagaaaagaaagaacaagaaataaagaagaaatttaaactaacaggccctattcaagtcatccatcttgcaaaagcttgttgtgatgtcaaaggaggaaagaatgaactgagcttcaagcaaggagagcaaattgaaatcatccgcatcacagacaacccagaaggaaaatggttgggcagaacagcaaggggttcatatggctatattaaaacaactgctgtagagattgactatgattctttgaaactgaaaaaagactctcttggtgccccttcaagacctattgaagatgaccaagaagtatatgatgatgttgcagagcaggatgatattagcagccacagtcagagtggaagtggagggatattccctccaccaccagatgatgacatttatgatgggattgaagaggaagatgctgatgatggtttccctgctcctcctaaacaattggacatgggagatgaagtttacgatgatgtggatacctctgatttccctgtttcatcagcagagatgagtcaaggaactaatgttggaaaagctaagacagaagaaaaggaccttaagaagctaaaaaagcaggaaaaagaagaaaaagacttcaggaaaaaatttaaatatgatggtgaaattagagtcctatattcaactaaagttacaacttccataacttctaaaaagtggggaaccagagatctacaggtaaaacctggtgaatctctagaagttatacaaaccacagatgacacaaaagttctctgcagaaatgaagaagggaaatatggttatgtccttcggagttacctagcggacaatgatggagagatctatgatgatattgctgatggctgcatctatgacaatgactagcactcaactttggtcattctgctgtgttcattaggtgccaatgtgaagtctggattttaattggcatgttattgggtatcaagaaaattaatgcacaaaaccacttattatcatttgttatgaaatcccaattatctttacaaagtgtttaaagtttgaacatagaaaataatctctctgcttaattgttaactcagaagactacattagtgagatgtaagaattattaaatattccatttccgctttggctacaattatgaagaagttgaaggtacttcttttagaccaccagtaaataatcctccttcaaaaaataaaaataaaagaaaaaggaaaatcattcaggaagaaatgacctgtctaaaaaaacctaaggaagaataataatataagaaaggaaatttaaaaacattccacaagaagaaaaattattgtttatacttctacttatggttatatcttatattctctattcaagtgacctgtcttttaaaaaggcagtgctgtcttacctcttgctagtgggttaaatgttttcaaaaattatagcagtagtagaagttttgtataaaatttgtccttatttgttaattgtatataaatgttaattatttgatacgaatgttatgcatttagtatgcacattgaagtctaaactgtagaagagtctaaaacaagttctctttttgcagattcacatactaatggtttaattctgtgctctgtttaaagtactattataactagagtagatctgaatgaggataaccctaaaatcatgaggaatggaagaatggaccttgaaactacctaggcttttatgcatggcacctctttataatgaagacactttttaaagtttttgtttttgtttcaattaccgctagatttttttttctctttttttaaaatccattttactggaaagttggccagcagagggagtagaaattattaaaattctagtgtttggattgggcccttctctaacagtacatactcattcccaaagcaatccaaaaacaaaatgtgaaccatttgggtttcaaatgttaagaacactaaatagcatgatttaaaaaatgaaaaatgctaacacccaagaaaagaagatattaagtgctttttaacaactcctagagtacaaaatgagtacatcataatgctggctcttctactaatgaaccatcgagtgatattgaataaattatttatcttctcagtttccttatctgtaaattacaatattagactaagtaagtttttccaactcttcactaccaattaccttaggcttttataatgctccgcctacttcagtcccatgtttcagaagcttttgtctattttttaaactcattgattaaataatgattaatgcattctccacattttaatattgcaaaggcccattggagtttctgaagtggctccacagaattgaaataatttcaaataactgtaaaggaactgaaaatcttcacagagatgaagtggggtttccattaggtgctttgaaatttgataacaaatcatcaacttccactggtcaatatatagattttgggtgtctgaggccccaagattagatgccactaatctccaaagattccctccaattatgaaatattttaatgtctacttttagagagcactagccagtatatgaccatgtgattaatttcttttcacactagataaaattacctggttcaaaagtggtttttgtttattaaatttggtaataaatatatataatacacagacaggatagtttttatgctgaagtttttggccagctttagtttgaggactccttgataagcttgctaaactttcagagtgccctgagacacttccagccatccctcctcctgccttcattggggcagacttgcattgcagtctgacagtaattttttttctgattgagaattatgtaaattcaatacaatgtcagtttttaaaagtcaaagttagatcaagagaatatttcagagttttggtttacacatcaagaaacagacacacatacctaggaaagatttacacaatagataatcatcttaatgtgaaagatatttgaagtattaattttaatatattaaatatgatttctgttatagtcttctgtatggaattttgtcacttaagatgagctgcaaataaataataccttcaatggaaaaaaaaaaaaaaaaaaaaaaaaaaaaaaaaaaaaaaaaaaaaaaaaa

SEQ ID NO: 12 - DNA, 3439 bp, Homo sapiens
acggcgcatgctccgcaatcatcttctttaccctggagctgctgctgctgctgctgcttttgcttttggggctgagtttaataagcgagcgagcgagcaagcgagcgcggggggaaaaaggcagagaatgtccgccatctaccctccgctcctgggcgcgctctcattcatagcagcctcttcatgaattacagctgagggggggcggaggaggggggggtaccacacaacaccccagcaaacctccgggcccccaggcatggctagctcgtgtgccgtgcaggtgaagctggagctggggcaccgcgcccaggtgaggaaaaaacccaccgtggagggcttcacccacgactggatggtgttcgtacgcggtccggagcacagtaacatacagcactttgtggagaaagtcgtcttccacttgcacgaaagctttcctaggccaaaaagagtgtgcaaagatccaccttacaaagtagaagaatctgggtatgctggtttcattttgccaattgaagtttattttaaaaacaaggaagaacctaggaaagtccgctttgattatgacttattcctgcatcttgaaggccatccaccagtgaatcacctccgctgtgaaaagctaactttcaacaaccccacagaggactttaggagaaagttgctgaaggcaggaggggaccctaataggagtattcataccagcagcagcagcagcagcagcagtagcagcagcagcagcagcagcagcagcagcagtagcagcagcagcagcagcagcagcagcagcagtagcagcagcagtagcagcagcagcagcagcagtagtaccagtttttcaaagcctcacaaattaatgaaggagcacaaggaaaaaccttctaaagactccagagaacataaaagtgccttcaaagaaccttccagggatcacaacaaatcttccaaagaatcctctaagaaacccaaagaaaataaaccactgaaagaagagaaaatagttcctaagatggccttcaaggaacctaaacccatgtcaaaagagccaaaaccagatagtaacttactcaccatcaccagtggacaagataagaaggctcctagtaaaaggccgcccatttcagattctgaagaactctcagccaaaaaaaggaaaaagagtagctcagaggctttatttaaaagtttttctagcgcaccaccactgatactcacttgttctgctgacaaaaaacagataaaagataaatctcatgtcaagatgggaaaggtcaaaattgaaagtgagacatcagagaagaagaaatcaacgttaccgccatttgatgatattgtggatcccaatgattcagatgtggaggagaatatatcctctaaatctgattctgaacaacccagtcctgccagctccagctccagctccagctccagcttcacaccatcccagaccaggcaacaaggtcctttgaggtctataatgaaagatctgcattctgatgacaatgaggaggaatcagatgaagtggaggataacgacaatgactctgaaatggagaggcctgtaaatagaggaggcagccgaagtcgcagagttagcttaagtgatggcagcgatagtgaaagcagttctgcttcttcacccctacatcacgaacctccaccacccttactaaaaaccaacaacaaccagattcttgaagtgaaaagtccaataaagcaaagcaaatcagataagcaaataaagaatggtgaatgtgacaaggcatacctagatgaactggtagagcttcacagaaggttaatgacattgagagaaagacacattctgcagcagatcgtgaaccttatagaagaaactggacactttcatatcacaaacacaacatttgattttgatctttgctcgctggacaaaaccacagtccgtaaactacagagttacctggaaacatctggaacatcctgaggatataacaactggatgcatcaagaactattgtgttttttttttttggttttttttttttttggttgtgattttttgttcttgttgtttatatgaaaacactcaaaatgatgcaaccaaaagggaaaaaataaaaatcaaacaaccttcagctttatttttctttaaagccagtcatcatctcttgataaaggagaggttaaagcaaaccagcctcagcggaccactcttctctccaaggaaatccccgggaagagttagcctggatagccttgaaaacaaacaaatcaaacacaacacaagaaaactcaaagaatgtgtatggtatcatgtatctctctgtggtggttcattccacaggacgaatgcatattcaacacactgccttattacataactgatctatttattatcgcatacagatattctaagtcgttgagggaatgacaccatcagacattataagtacttggtcccgtggatgctctttcaatgcagcacccttgccatcccaagcccagtgaccttactcgtataccgtgccactttccaccaactttttccaagtcctttaactcgttgcagtctgtattttccaccttttgtttttccagttccaggacacagattatcaactggggggaccaaatagccaccttgattttcttctttgtggtctttttcctgaaagttggggcccagtccttggctgtatccatgtaatgatcttggaccatggtagaaaatgcaccaaataggatcatatgaattgctgtctagccttagtcaataaacttgtaggacttttaaacaaaagtgtacctgtaaatgtcctgaatccagcattgttgagctgtcatcaacattcttgtgtctgttttactgttacaatattaggtgaatatggaagtaaaggcattccacaggatcatcatttaaaaaaaaagaattctggtcctgttttctaaaaaaaaaaactgttgtagaaattcttaatttggatctatttattagtcagagtttcagctttcttcagctgccagtgtgttactcatctttatcctaaaaatctggaatcagagatttttgtttgttcacatatgattctcttagacacttttatatttgaaaaaattaaaatctttctttggggaaaaattcttggttattctgccataacagattatgtattaacttgtagattcagtggttcaatacctgtttagttgcttgctaatatttccagaaggatttcttgtattggtgaaagacggttggggatggggggatttttttgttcttgttgtacccttgttttgaaactagaaatctgtcctgtggcatgcaaaagaaagcaaattatttttaaaagaaaaaaaccaaagtacttttggtgtcattattccatcttctccataagtggagaaatgaaaagtaagaacagctcatcttcaaagtttttactagaaattc

SEQ ID NO: 13 - protein, 568 aa, Homo sapiens
MASSCAVQVKLELGHRAQVRKKPTVEGFTHDWMVFVRGPEHSNIQHFVEKVVFHLHESFPRPKRVCKDPPYKVEESGYAGFILPIEVYFKNKEEPRKVRFDYDLFLHLEGHPPVNHLRCEKLTFNNPTEDFRRKLLKAGGDPNRSIHTSSSSSSSSSSSSSSSSSSSSSSSSSSSSSSSSSSSSSSSSSSTSFSKPHKLMKEHKEKPSKDSREHKSAFKEPSRDHNKSSKESSKKPKENKPLKEEKIVPKMAFKEPKPMSKEPKPDSNLLTITSGQDKKAPSKRPPISDSEELSAKKRKKSSSEALFKSFSSAPPLILTCSADKKQIKDKSHVKMGKVKIESETSEKKKSTLPPFDDIVDPNDSDVEENISSKSDSEQPSPASSSSSSSSSFTPSQTRQQGPLRSIMKDLHSDDNEEESDEVEDNDNDSEMERPVNRGGSRSRRVSLSDGSDSESSSASSPLHHEPPPPLLKTNNNQILEVKSPIKQSKSDKQIKNGECDKAYLDELVELHRRLMTLRERHILQQIVNLIEETGHFHITNTTFDFDLCSLDKTTVRKLQSYLETSGTS

SEQ ID NO: 14 - DNA, 3122 bp, Homo sapiens
tcggcggagacctgctccccagaagacgcctcctgcttcccactgcgccctggaggacgcgggctggctgctgggcgagctcggcggaggcacgcccctcgcctccccgcggagtgcggactcgccccggtgcccaaactccgcccaccctctagggagctccgctctcccgcctaaccccggcactccggacagagctgggcctggggaaggggttcctgaactacgcggacgccgaacgggacgcgctgcagaagcgcacgagtctgcggccacgcgcgctccgatggctgccaggagctgagctcagggtgggcggaggaagcggttagacgccccgaaactgagctgcacgtttctaaggtagggaggaggaagatgcccccaattaagttgatctttgagccaaggaggctggggagcagcctccccaagctagagccctgcagagcgagtttcccttgacctcgctgcgcctctggcgcgctctgcagcgcggacccgcggcccctcgggaaagcgcagtcggaaagttatccgcggcggttccctgcgcgccctgttgtgtaagctcggcgttgccagcggacggagaagttgctggcttgcccgatagcccagttcggtggcggcccggggcggatttcatggcccgcggcgaacgcggggccagagctggcgtgggcgagcccctgcgcgccccctcccgcggggatccagttcgcctgctcccttccgctcgctggcttttccgatgcttgctgcgcccctggccgccgctgccctctcgccgcctcctacccctcggagccgccgcctaagtcgaggaggagagaatgaccgaggtgctgtggccggctgtccccaacgggacggacgctgccttcctggccggtccgggttcgtcctgggggaacagcacggtcgcctccactgccgccgtctcctcgtcgttcaaatgcgccttgaccaagacgggcttccagttttactacctgccggctgtctacatcttggtattcatcatcggcttcctgggcaacagcgtggccatctggatgttcgtcttccacatgaagccctggagcggcatctccgtgtacatgttcaatttggctctggccgacttcttgtacgtgctgactctgccagccctgatcttctactacttcaataaaacagactggatcttcggggatgccatgtgtaaactgcagaggttcatctttcatgtgaacctctatggcagcatcttgtttctgacatgcatcagtgcccaccggtacagcggtgtggtgtaccccctcaagtccctgggccggctcaaaaagaagaatgcgatctgtatcagcgtgctggtgtggctcattgtggtggtggcgatctcccccatcctcttctactcaggtaccggggtccgcaaaaacaaaaccatcacctgttacgacaccacctcagacgagtacctgcgaagttatttcatctacagcatgtgcacgaccgtggccatgttctgtgtccccttggtgctgattctgggctgttacggattaattgtgagagctttgatttacaaagatctggacaactctcctctgaggagaaaatcgatttacctggtaatcattgtactgactgtttttgctgtgtcttacatccctttccatgtgatgaaaacgatgaacttgagggcccggcttgattttcagaccccagcaatgtgtgctttcaatgacagggtttatgccacgtatcaggtgacaagaggtctagcaagtctcaacagttgtgtggaccccattctctatttcttggcgggagatactttcagaaggagactctcccgagccacaaggaaagcttctagaagaagtgaggcaaatttgcaatccaagagtgaagacatgaccctcaatattttacctgagttcaagcagaatggagatacaagcctgtgaaggcacaagaatctccaaacacctctctgttgtaatatggtaggatgcttaacagaatcaagtacttttcccctctttaactttctagtttagaaaaaaatcaaaccaagaaaatagtgagttaaaaaaataatagaagtagaaatgcccacatccacacttagcttgtttgggtttgctttcacagtctctcttccttctgactagaagtatgtataataaaacaatactacctagttaaacatttactttctcttttgcctttaaaatgtgcaggcttttctgtttaaagtgtgtgtgcacatgagtactggggctgtttttgatattagtaatttctctaagaaaactagccccctgcaacttgagtttgtggtttatctagcctttattgtttttttaaaatccacagtaggaataaaaaatctatattctcagaaatatctagcatggtatataacaaaacactaaactcatcagttcatccggcatcagatcaatggatctctgagcggggtgtttttttcagtgtcttataagcatagatgatagttgactgagtttctttagggcattgaatagacaagtaaagctaatgaatttaaaagcctgaaaagtgattgttttccagttatttctggaaaaggtctcattatatattgggtgctaaatgtttgatggggaaagcctgcatatattatcgtactggtaaaatgcattcaaaataattaaagtgcatgtattttccttgtaaacaccatgagctctcttagacatcttgtgataaagagcatttacttgccccactgctgtgcaatgccttaggactttgtttgtgttccaggacaagtgttcactcacatctgtaaaaacaattttaagaattgcaaataaattacagaccaaagattgagtaaagtcaaataactgttagtaagttgaaggatattggacaggaggacagtatttcagaaaaggagaggttgacagtcatccacaaggcatagcctccaagtatactctcaaatgtatgaagcaactggggtgggcagaagacattttagaatgagggctttagtttaaattaaagtcatggtggagaagactcttgcttcctccaagtgtttgaaaacacaaaatgcgatatgaaaaaaaaaaaaaaaa

SEQ ID NO: 15 - protein, 373 aa, Homo sapiens
MTEVLWPAVPNGTDAAFLAGPGSSWGNSTVASTAAVSSSFKCALTKTGFQFYYLPAVYILVFIIGFLGNSVAIWMFVFHMKPWSGISVYMFNLALADFLYVLTLPALIFYYFNKTDWIFGDAMCKLQRFIFHVNLYGSILFLTCISAHRYSGVVYPLKSLGRLKKKNAICISVLVWLIVVVAISPILFYSGTGVRKNKTITCYDTTSDEYLRSYFIYSMCTTVAMFCVPLVLILGCYGLIVRALIYKDLDNSPLRRKSIYLVIIVLTVFAVSYIPFHVMKTMNLRARLDFQTPAMCAFNDRVYATYQVTRGLASLNSCVDPILYFLAGDTFRRRLSRATRKASRRSEANLQSKSEDMTLNILPEFKQNGDTSL

SEQ ID NO: 16 - protein, 15 aa, Artificial Sequence
  A synthetic polypeptide
GGGGSGGGGSGGGGS